(19) United States
(12) Patent Application Publication
(10) Pub. No.: US 2024/0325506 A1
(43) Pub. Date: Oct. 3, 2024

(54) COMPOSITIONS AND METHODS FOR TREATMENT OF GAUCHER DISEASE

(71) Applicant: AvroBio, Inc., Cambridge, MA (US)

(72) Inventors: Mark DEANDRADE, Boston, MA (US); Chris MASON, Cambridge, MA (US); Chris MASON, Nashua, NH (US)

(21) Appl. No.: 18/573,086

(22) PCT Filed: Jun. 23, 2022
(86) PCT No.: PCT/US2022/034714

§ 371 Date: Dec. 21, 2023

Related U.S. Application Data
(60) Provisional application No. 63/213,885, filed on Jun. 23, 2021.

(51) Int. Cl. A61K 38/47; A61K 38/17; A61K 48/00; A61P 3/00; C12N 15/113

(52) U.S. Cl. CPC A61K 38/47; A61K 38/1774; A61K 48/0033; A61K 48/005; A61P 3/00; C12N 15/1137; C12N 15/1138; C12N 2310/14; C12N 2310/141; C12N 2310/531

(57) ABSTRACT

Described herein are methods for treating a subject having or at risk of developing Gaucher disease, by administering one or more agents that increase expression and/or activity of glucocerebrosidase (GBA) and/or scavenger receptor class B member 2 (SCARB2), such as pluripotent cells that express GBA and/or SCARB2, and to the subject. Also disclosed are compositions comprising one or more agents that increase the expression and/or activity of GBA and/or SCARB2.

### SEQUENCE LISTING

The instant application contains a Sequence Listing which has been submitted electronically in ASCII format and is hereby incorporated by reference in its entirety. The ASCII copy, created on Jun. 21, 2022, is named 51182-034WO2_Sequence_Listing_6_21_22_ST25 and is 76,368 bytes in size.

### FIELD OF THE INVENTION

The invention relates to compositions and methods for the treatment of Gaucher disease.

### BACKGROUND

Gaucher disease is an autosomal recessive lysosomal storage disorder caused by a deficiency in the glucocerebrosidase (GBA) gene, resulting in accumulation of GBA in lysosomes of phagocytic cells of the liver, spleen, and bone marrow of affected patients. Gaucher patients clinically present with splenomegaly, hepatomegaly, bone disease, thrombocytopenia, and anemia. Known treatment modalities for patients with Gaucher disease have generally relied on ameliorating the symptoms of the disease without addressing the underlying cause. Enzyme replacement therapy (ERT) for GBA has produced some benefit to Gaucher patients; however, the benefits of ERT are short-lived, thereby requiring frequent administration of recombinant GBA, which is costly and negatively impacts patient compliance. Thus, there is an urgent need for treatment of Gaucher disease that addresses the underlying physiological cause of the disease while providing long-term relief to the patient.

### SUMMARY OF THE INVENTION

The present invention provides methods for treating Gaucher disease using one or more agents that increase expression and/or activity of both β-glucocerebrosidase (GBA) and scavenger receptor class B member 2 (SCARB2). Exemplary agents that may be used in conjunction with the compositions and methods of the disclosure are one or more polynucleotides including a transgene that encodes a GBA protein and/or a SCARB2 protein, one or more interfering RNA (RNAi) molecules that collectively increase expression and/or activity of the GBA and/or SCARB2 protein, a GBA and/or a SCARB2 protein, and one or more small molecules that collectively increase expression and/or activity of the GBA and/or SCARB2 proteins. Also disclosed are pluripotent cells, such as CD34+ cells and hematopoietic stem cells, among others, expressing one or more polynucleotides capable of collectively increasing the expression and/or activity of GBA and/or SCARB2. The agents may be administered to a patient having Gaucher disease by one or more of a variety of routes, including directly to the central nervous system of the patient (e.g., by intracerebroventricular administration) or systemically (e.g., by intravenous administration), among others.

In a first aspect, the disclosure provides a method of treating a subject diagnosed as having or at risk of developing Gaucher disease by providing to the subject one or more (e.g., 2, 3, 4, or more) agents that collectively increase expression and/or activity GBA and SCARB2.

In some embodiments, the one or more (e.g., 2, 3, 4, or more) agents comprise a first agent that increases expression and/or activity of GBA and a second agent that increases expression and/or activity of SCARB2. For example, the first agent may comprise (i) one or more (e.g., 2, 3, 4, or more) polynucleotides comprising a transgene that encodes a GBA protein, (ii) one or more (e.g., 2, 3, 4, or more) interfering RNAi molecules that collectively increase expression and/or activity of the GBA protein, (iii) one or more (e.g., 2, 3, 4, or more) polynucleotides encoding the one or more RNAi molecules that collectively increase expression and/or activity of the GBA protein, (iv) a GBA protein, or (v) one or more (e.g., 2, 3, 4, or more) small molecules that collectively increase expression and/or activity of the GBA protein, and the second agent comprises (vi) one or more (e.g., 2, 3, 4, or more) polynucleotides comprising a transgene that encodes a SCARB2 protein, (vii) one or more (e.g., 2, 3, 4, or more) RNAi molecules that collectively increase expression and/or activity of the SCARB2 protein, (viii) one or more (e.g., 2, 3, 4, or more) polynucleotides encoding the one or more RNAi molecules that collectively increase expression and/or activity of the SCARB2 protein, (ix) a SCARB2 protein, and/or (x) one or more (e.g., 2, 3, 4, or more) small molecules that collectively increase expression and/or activity of the SCARB2 protein.

In some embodiments, the GBA protein has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO: 1. In some embodiments, the GBA protein has an amino acid sequence that is at least 90% (e.g., at least 91%, 92%, 93%, 94%, 95%, 96%, 97%, 98%, 99%, or more) identical to the amino acid sequence of SEQ ID NO: 1; optionally, wherein the GBA has an amino acid sequence that is at least 95% (e.g., at least 96%, 97%, 98%, 99%, or more) identical to the amino acid sequence of SEQ ID NO: 1; optionally, wherein the GBA has the amino acid sequence of SEQ ID NO: 1.

In some embodiments, the GBA has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO: 5. In some embodiments, the GBA has an amino acid sequence that is at least 90% (e.g., at least 91%, 92%, 93%, 94%, 95%, 96%, 97%, 98%, 99%, or more) identical to the amino acid sequence of SEQ ID NO: 5; optionally, wherein the GBA has an amino acid sequence that is at least 95% (e.g., at least 96%, 97%, 98%, 99%, or more) identical to the amino acid sequence of SEQ ID NO: 5; optionally; wherein the GBA has the amino acid sequence of SEQ ID NO: 5.

In some embodiments, the transgene encoding GBA has a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO: 6. In some embodiments, the transgene encoding GBA has a nucleic acid sequence that is at least 90% (e.g., at least 91%, 92%, 93%, 94%, 95%, 96%, 97%, 98%, 99%, or more) identical to the nucleic acid sequence of SEQ ID NO: 6; optionally, wherein the transgene encoding GBA has a nucleic acid sequence that is at least 95% (e.g., at least 96%, 97%, 98%, 99%, or more) identical to the nucleic acid sequence of SEQ ID NO: 6; optionally, wherein the transgene encoding GBA has the nucleic acid sequence of SEQ ID NO: 6.

In some embodiments, the transgene encoding GBA has a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO: 7. In some embodiments, the transgene encoding GBA has a nucleic acid sequence that is at least 90% (e.g., at least 91%, 92%, 93%, 94%, 95%, 96%, 97%, 98%, 99%, or more) identical to the nucleic acid sequence of SEQ ID NO: 7; optionally, wherein the transgene encoding GBA has a nucleic acid sequence that is at least 95% (e.g., at least 96%, 97%, 98%, 99%, or more) identical to the nucleic acid sequence of SEQ ID NO: 7; optionally, wherein the transgene encoding GBA has the nucleic acid sequence of SEQ ID NO: 7.

In some embodiments, the transgene encoding GBA has a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO: 11. In some embodiments, the transgene encoding GBA has a nucleic acid sequence that is at least 90% (e.g., at least 91%, 92%, 93%, 94%, 95%, 96%, 97%, 98%, 99%, or more) identical to the nucleic acid sequence of SEQ ID NO: 11; optionally, wherein the transgene encoding GBA has a nucleic acid sequence that is at least 95% (e.g., at least 96%, 97%, 98%, 99%, or more) identical to the nucleic acid sequence of SEQ ID NO: 11; optionally, wherein the transgene encoding GBA has the nucleic acid sequence of SEQ ID NO: 11, sequence that is at least 85% identical to the nucleic acid sequence of SEQ ID NO: 11.

In some embodiments, the GBA comprises a signal peptide. The signal peptide may have, for example, an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO: 12. In some embodiments, the signal peptide has an amino acid sequence that is at least 90% (e.g., at least 91%, 92%, 93%, 94%, 95%, 96%, 97%, 98%, 99%, or more) identical to the amino acid sequence of SEQ ID NO: 12; optionally, wherein the signal peptide has an amino acid sequence that is at least 95% (e.g., at least 96%, 97%, 98%, 99%, or more) identical to the amino acid sequence of SEQ ID NO: 12; optionally, wherein the signal peptide has the amino acid sequence of SEQ ID NO: 12.

In some embodiments, the GBA comprises a signal peptide that is encoded by a polynucleotide that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO: 13. In some embodiments, the signal peptide is encoded by a polynucleotide that is at least 90% (e.g., at least 91%, 92%, 93%, 94%, 95%, 96%, 97%, 98%, 99%, or more) identical to the nucleic acid sequence of SEQ ID NO: 13; optionally, wherein the signal peptide is encoded by a polynucleotide that is at least 95% (e.g., at least 96%, 97%, 98%, 99%, or more) identical to the nucleic acid sequence of SEQ ID NO: 13; optionally, wherein the signal peptide is encoded by a polynucleotide having the nucleic acid sequence of SEQ ID NO: 13.

In some embodiments, the SCARB2 has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO: 14. In some embodiments, the SCARB2 has an amino acid sequence that is at least 90% (e.g., at least 91%, 92%, 93%, 94%, 95%, 96%, 97%, 98%, 99%, or more) identical to the amino acid sequence of SEQ ID NO: 14; optionally, wherein the SCARB2 has an amino acid sequence that is at least 95% (e.g., at least 96%, 97%, 98%, 99%, or more) identical to the amino acid sequence of SEQ ID NO: 14; optionally, wherein the SCARB2 has an amino acid sequence of SEQ ID NO: 14.

In some embodiments, the SCARB2 has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO: 15. In some embodiments, the SCARB2 has an amino acid sequence that is at least 90% (e.g., at least 91%, 92%, 93%, 94%, 95%, 96%, 97%, 98%, 99%, or more) identical to the amino acid sequence of SEQ ID NO: 15; optionally, wherein the SCARB2 has an amino acid sequence that is at least 95% (e.g., at least 96%, 97%, 98%, 99%, or more) identical to the amino acid sequence of SEQ ID NO: 15; optionally, wherein the SCARB2 has an amino acid sequence of SEQ ID NO: 15.

In some embodiments, the polynucleotide encoding SCARB2 has a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO: 16. In some embodiments, the polynucleotide encoding SCARB2 has a nucleic acid sequence that is at least 90% (e.g., at least 91%, 92%, 93%, 94%, 95%, 96%, 97%, 98%, 99%, or more) identical to the nucleic acid sequence of SEQ ID NO: 16; optionally, wherein the polynucleotide encoding SCARB2 has a nucleic acid sequence that is at least 95% (e.g., at least 96%, 97%, 98%, 99%, or more) identical to the nucleic acid sequence of SEQ ID NO: 16; optionally, wherein the polynucleotide encoding SCARB2 has a nucleic acid sequence of SEQ ID NO: 16.

In some embodiments, the polynucleotide encoding SCARB2 has a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO: 17. In some embodiments, the polynucleotide encoding SCARB2 has a nucleic acid sequence that is at least 90% (e.g., at least 91%, 92%, 93%, 94%, 95%, 96%, 97%, 98%, 99%, or more) identical to the nucleic acid sequence of SEQ ID NO: 17; optionally, wherein the polynucleotide encoding SCARB2 has a nucleic acid sequence that is at least 95% (e.g., at least 96%, 97%, 98%, 99%, or more) identical to the nucleic acid sequence of SEQ ID NO: 17; optionally, wherein the polynucleotide encoding SCARB2 has a nucleic acid sequence of SEQ ID NO: 17.

In some embodiments, the SCARB2 comprises a signal peptide. The signal peptide may have, for example, an amino acid sequence that is at least 70% identical to the amino acid sequence of SEQ ID NO: 49; optionally, wherein the signal peptide has an amino acid sequence that is at least 75% (e.g., at least 76%, 80%, 85%, 90%, 95%, 96%, 97%, 98%, 99%, or more) identical to the amino acid sequence of SEQ ID NO: 49; optionally, wherein the signal peptide has an amino acid sequence that is at least 80% (e.g., at least 81%, 85%, 90%, 95%, 96%, 97%, 98%, 99%, or more) identical to the amino acid sequence of SEQ ID NO: 49; optionally, wherein the signal peptide has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO: 49; optionally, wherein the signal peptide has an amino acid sequence that is at least 90% (e.g., at least 91%, 92%, 93%, 94%, 95%, 96%, 97%, 98%, 99%, or more) identical to the amino acid sequence of SEQ ID NO: 49; optionally, wherein the signal peptide has an amino acid sequence that is at least 95% (e.g., at least 96%, 97%, 98%, 99%, or more) identical to the amino acid sequence of SEQ ID NO: 49; optionally, wherein the signal peptide has an amino acid sequence of SEQ ID NO: 49.

In some embodiments, the SCARB2 is a GBA-binding domain of SCARB2. In some embodiments, the GBA-binding domain of the SCARB2 protein has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO: 18; optionally, wherein the GBA-binding domain of the SCARB2 protein has an amino acid sequence that is at least 90% (e.g., at least 91%, 92%, 93%, 94%, 95%, 96%, 97%, 98%, 99%, or more) identical to the amino acid sequence of SEQ ID NO: 18; optionally, wherein the GBA-binding domain of the SCARB2 protein has an amino acid sequence that is at least 95% (e.g., at least 96%, 97%, 98%, 99%, or more) identical to the amino acid sequence of SEQ ID NO: 18; optionally, wherein the GBA-binding domain of the SCARB2 protein has an amino acid sequence of SEQ ID NO: 18.

In some embodiments, the GBA-binding domain of the SCARB2 protein has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO: 19; optionally, wherein the GBA-binding domain of the SCARB2 protein has an amino acid sequence that is at least 90% (e.g., at least 91%, 92%, 93%, 94%, 95%, 96%, 97%, 98%, 99%, or more) identical to the amino acid sequence of SEQ ID NO: 19; optionally, wherein the GBA-binding domain of the SCARB2 protein has an amino acid sequence that is at least 95% (e.g., at least 96%, 97%, 98%, 99%, or more) identical to the amino acid sequence of SEQ ID NO: 19; optionally, wherein the GBA-binding domain of the SCARB2 protein has an amino acid sequence of SEQ ID NO: 19.

In some embodiments, the GBA and/or SCARB2 is a fusion protein comprising GBA or SCARB2 and a glycosylation independent lysosomal targeting (GILT) tag. In some embodiments, the GILT tag comprises a human IGF-II mutein having an amino acid sequence that is at least 70% (e.g., at least 75%, 80%, 85%, 90%, 95%, 96%, 97%, 98%, 99%, or more) identical to the amino acid sequence of mature human IGF-II (SEQ ID NO: 22), and having diminished binding affinity for the insulin receptor relative to the affinity of naturally-occurring human IGF-II for the insulin receptor, wherein the IGF-II mutein is resistant to furin cleavage and binds to the human cation-independent mannose-6-phosphate receptor in a mannose-6-phosphate-independent manner.

In some embodiments, the IGF-II mutein comprises a mutation within a region corresponding to amino acids 30-40 of SEQ ID NO: 22, and wherein the mutation abolishes at least one furin protease cleavage site. In some embodiments, the mutation is an amino acid substitution, deletion, and/or insertion. In some embodiments, the mutation is an Ala amino acid substitution at a position corresponding to Arg37 of SEQ ID NO: 22. In some embodiments, the mutation is a deletion or replacement of amino acid residues corresponding to positions selected form the group consisting of 31-40, 32-40, 33-40, 34-40, 30-39, 31-39, 32-39, 34-37, 33-39, 35-39, 36-39, 37-40, 34-40 of SEQ ID NO: 22, and combinations thereof.

In some embodiments, the GILT tag is a polypeptide having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 23. In some embodiments, the GILT tag is a polypeptide having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 24. In some embodiments, the GILT tag is a polypeptide having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 25. In some embodiments, the GILT tag is a polypeptide encoded by a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 26. In some embodiments, the GILT tag is a polypeptide encoded by a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 27. In some embodiments, the GILT tag is a polypeptide encoded by a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 28.

In some embodiments, the GBA fusion protein and/or the SCARB2 fusion protein comprises a receptor-binding (Rb) domain of apolipoprotein E (ApoE). In some embodiments, the GBA fusion protein comprises a Rb domain of ApoE. In some embodiments, the SCARB2 fusion protein comprises a Rb domain of ApoE. In some embodiments, the Rb domain comprises a portion of ApoE having the amino acid sequence of residues 25-185, 50-180, 75-175, 100-170, 125-160, or 130-150 of SEQ ID NO: 29. In some embodiments, the Rb domain comprises a region having at least 70% (e.g., at least 71%, 75%, 80%, 85%, 90%, 91%, 92%, 93%, 94%, 95%, 96%, 97%, 98%, 99% or more) sequence identity to the amino acid sequence of residues 159-167 of SEQ ID NO: 29.

In some embodiments, the GBA protein or the SCARB2 protein is a fusion protein comprising GBA or SCARB2 and a cell-penetrating peptide (CPP). In some embodiments, the GBA protein is a fusion protein comprising a CPP. In some embodiments, the SCARB2 protein is a fusion protein comprising a CPP. In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of any one of SEQ ID NOs: 30-48; optionally, wherein the CPP has an amino acid sequence having at least 90% (e.g., at least 91%, 92%, 93%, 94%, 95%, 96%, 97%, 98%, 99%, or more) sequence identity to the amino acid sequence of any one of SEQ ID NOs: 30-48; optionally, wherein the CPP has an amino acid sequence having at least 95% (e.g., at least 96%, 97%, 98%, 99%, or more) sequence identity to the amino acid sequence of any one of SEQ ID NOs: 30-48; optionally, wherein the CPP has an amino acid sequence having the amino acid sequence of any one of SEQ ID NOs: 30-48.

In some embodiments, the transgene encoding GBA and/or SCARB2 further comprises a microRNA (miRNA) targeting sequence in the 3′-UTR. In some embodiments, the miRNA targeting sequence is a miR-126 targeting sequence.

In some embodiments, the GBA and/or the SCARB2 penetrates the blood brain barrier (BBB) in the subject.

In some embodiments, the one or more RNAi molecules comprise short interfering RNA (siRNA), short hairpin RNA (shRNA), and/or microRNA (miRNA).

In some embodiments, the one or more polynucleotides are provided to the subject by administering to the subject a composition comprising a population of cells that together contain nucleic acids encoding the GBA and/or SCARB2 protein. In some embodiments, the population is a uniform population of cells that contain nucleic acids encoding the proteins or a heterogeneous population of cells that together contain nucleic acids encoding the GBA and/or SCARB2 protein.

In some embodiments, the cells are pluripotent cells or multipotent cells. In some embodiments, the multipotent cells are CD34+ cells. In some embodiments, the CD34+ cells are hematopoietic stem cells (HSCs) or myeloid progenitor cells (MPCs). In some embodiments, the pluripotent cells are embryonic stem cells (ESCs) or induced pluripotent stem cells (iPSCs). In some embodiments, the cells are blood lineage progenitor cells (BLPCs), microglial progenitor cells, monocytes, macrophages, or microglia. In some embodiments, the BLPCs are monocytes.

In some embodiments, a population of endogenous hematopoietic cells in the subject has been ablated prior to administration of the one or more agents to the subject, optionally wherein the hematopoietic cells are CD34+ cells, BLPCs, microglial progenitor cells, monocytes, macrophages, or microglia. In some embodiments, the method comprising ablating a population of endogenous hematopoietic cells in the subject prior to administering the one or more agents to the subject, optionally wherein the hematopoietic cells are CD34+ cells, BLPCs, microglial progenitor cells, monocytes, macrophages, or microglia. In some embodiments, the microglia are ablated using an agent selected from the group consisting of busulfan, PLX3397, PLX647, PLX5622, treosulfan, and clodronate liposomes, by radiation therapy, or a combination thereof.

In some embodiments, the one or more agents is administered systemically to the subject. In some embodiments, the one or more agents is administered to the subject by way of intravenous injection. In some embodiments, the one or more agents is administered directly to the central nervous system of the subject. In some embodiments, the one or more agents is administered to the subject by way of intracerebroventricular injection, stereotactic injection, or a combination thereof. In some embodiments, the one or more agents is administered directly to the bone marrow of the subject. In some embodiments, the one or more agents is administered to the subject by way of intraosseous injection.

In some embodiments, the cells are autologous cells or allogeneic cells.

In some embodiments, the cells are transfected or transduced ex vivo to express the GBA and/or SCARB2. In some embodiments, the cells are transduced with a viral vector, such as a viral vector selected from the group consisting of a Retroviridae family virus, an adeno-associated virus (AAV), an adenovirus, a parvovirus, a coronavirus, a rhabdovirus, a paramyxovirus, a picornavirus, an alphavirus, a herpes virus, and a poxvirus. In some embodiments, the viral vector is a Retroviridae family viral vector. In some embodiments, the Retroviridae family viral vector is a lentiviral vector. In some embodiments, the Retroviridae family viral vector is an alpharetroviral vector. In some embodiments, the Retroviridae family viral vector is a gammaretroviral vector. In some embodiments, the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. In some embodiments, the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

In some embodiments, the pluripotent cells are transduced to express the GBA and SCARB2 from separate monocistronic expression cassettes. In some embodiments, the pluripotent cells are transduced to express the GBA and SCARB2 from a polycistronic expression cassette. In some embodiments, the pluripotent cells are transduced to express the GBA and SCARB2 from a bicistronic expression cassette.

In some embodiments, the polycistronic expression cassette comprises an internal ribosomal entry site (IRES) positioned between a polynucleotide encoding the GBA and a polynucleotide encoding the SCARB2. In some embodiments, the polycistronic expression cassette comprises a 2A polynucleotide positioned between a polynucleotide encoding the GBA and a polynucleotide encoding the SCARB2. In some embodiments, the 2A polynucleotide comprises a F2A, P2A, E2A, or T2A polynucleotide. In some embodiments, the 2A polynucleotide comprises a F2A polynucleotide. In some embodiments, the 2A polynucleotide comprises a P2A polynucleotide. In some embodiments, the 2A polynucleotide comprises a E2A polynucleotide. In some embodiments, the 2A polynucleotide comprises a T2A polynucleotide.

In some embodiments, one or more of the polynucleotides comprises a transgene encoding one or more (e.g., 1, 2, or more) of the proteins operably linked to a ubiquitous promoter, a cell lineage-specific promoter, or a synthetic promoter. In some embodiments, the ubiquitous promoter is selected from the group consisting of an elongation factor 1-alpha (EF1α) promoter, phosphoglycerate kinase 1 (PGK) promoter, or EF1α promoter containing elements of locus control region of the β-globin gene containing regions of erythroid-specific DNase I hypersensitivity (HS) regions 2, 3, and 4 (3-LCR(HS4,3,2)-EFS promoter). In some embodiments, the cell lineage-specific promoter is selected from the group consisting of a CD68 molecule (CD68) promoter, CD11 b molecule (CD11 b) promoter, C-X3-C motif chemokine receptor 1 (CX3CR1) promoter, allograft inflammatory factor 1 promoter (AIF1) promoter, purinergic receptor P2Y12 (P2Y12) promoter, transmembrane protein 119 (TMEM119) promoter, or colony stimulating factor 1 receptor (CSF1R) promoter. In some embodiments, the synthetic promoter is a Myeloproliferative Sarcoma Virus Enhancer, Negative Control Region Deleted, dl587rev Primer-Binding Site Substituted (MND) promoter. In some embodiments, the MND promoter comprises a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 20.

In some embodiments, the MND promoter comprises a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 21.

In some embodiments, the Gaucher disease is associated with one or more (e.g., 1, 2, 3, 4, 5, 6, 7, 9, 10, or more) mutations in the GBA gene. In some embodiments, the one or more mutations in the GBA gene comprise a p.N370S substitution, p.R463C substitution, p.L444P substitution, p.D409H substitution, p.R463C substitution, p.R496H substitution, p. F252I substitution, p.A456P substitution, p.V460V substitution, p.V394L, p.E326K substitution, p.G377S substitution, p.N188S substitution, c.84insG insertion, c.84dupG (84GG) duplication, c.115+1G>A substitution, or c.IVS2DS+1G-A splice site mutation.

In some embodiments, the Gaucher disease is Type 1 Gaucher disease. In some embodiments, the subject has a confirmed diagnosis of Type 1 Gaucher disease based on genotyping, deficient GBA activity in the blood (e.g., leukocytes, peripheral blood mononuclear cells, monocytes, among other blood components) of the subject, and/or clinical phenotype. In some embodiments, the deficient GBA activity in the subject is defined as activity that is equal to or greater than 15% (e.g., 15%, 20%, 25%, 30%, 35%, 40%, 45%, 50%, 55%, 60%, 65%, 70%, 75%, 80%, or more) of activity of GBA in a control reference patient not diagnosed as having Gaucher disease.

In some embodiments, the Gaucher disease is Type 2 Gaucher disease.

In some embodiments, the Gaucher disease is Type 3 Gaucher disease.

In some embodiments, the Gaucher disease is associated with one or more mutations in the SCARB2 gene. In some embodiments, the one or more mutations in the SCARB2 gene comprise a p.Q471 G substitution, p.H363N substitution, p.Q288Ter nonsense mutation, p.W178Ter nonsense mutation, p.W146fs frameshift mutation, p.Glu420fs frameshift mutation, g.76168478T>G transversion, g.1239+1G-T splice site mutation, or g.76168401dup splice site mutation.

In some embodiments, the subject is a human.

In some embodiments, the subject has undergone enzyme replacement therapy (ERT) comprising a total monthly dose of GBA ERT that is greater than 30 U/kg and less than 120 U/kg (e.g., 31, 35, 40, 45, 50, 55, 60, 65, 70, 75, 80, 85, 90, 95, 100, 105, 110, 115, or 119 U/kg) for 24 or more (e.g., 24, 25, 26, 27, 28, 29, 30, 31, 32, 33, 34, 35, 36, 37, 38, 39, 40, 41, 42, 43, 44, 45, 46, 47, 48, or more) consecutive months at a time of treatment with the one or more agents. In some embodiments, the subject has received a biweekly dose of GBA ERT greater than or equal to 15 U/kg and less than or equal to 60 U/kg (e.g., 31, 35, 40, 45, 50, 55, or 59 U/kg). In some embodiments, the subject has received a weekly dose of GBA ERT greater than or equal to 7.5 U/kg and less than or equal to 30 U/kg (e.g., 7.6, 8, 9, 10, 11, 12, 13, 14, 15, 16, 17, 18, 19, 20, 21, 22, 23, 24, 25, 26, 27, 28, or 29 U/kg).

In some embodiments, the subject has received substrate reduction therapy (SRT) for Gaucher disease during the 24 months immediately preceding treatment with the one or more agents. In some embodiments, the subject has not received SRT for Gaucher disease during the 24 months immediately preceding treatment with the one or more agents.

Additional embodiments of the present invention are listed in the enumerated paragraphs below.

E1. A method of treating a subject diagnosed as having or at risk of developing Gaucher's disease, the method comprising providing to the subject one or more agents that collectively increase expression and/or activity of β-glucocerebrosidase (GBA) and scavenger receptor class B member 2 (SCARB2).

E2. The method of E1, wherein the one or more agents comprise a first agent that increases expression and/or activity of GBA and a second agent that increases expression and/or activity of SCARB2, optionally wherein the first agent comprises (i) one or more polynucleotides comprising a transgene that encodes a GBA protein, (ii) one or more interfering RNA (RNAi) molecules that collectively increase expression and/or activity of the GBA protein, (iii) one or more polynucleotides encoding the one or more RNAi molecules that collectively increase expression and/or activity of the GBA protein, (iv) a GBA protein, or (v) one or more small molecules that collectively increase expression and/or activity of the GBA protein, and the second agent comprises (vi) one or more polynucleotides comprising a transgene that encodes a SCARB2 protein, (vii) one or more RNAi molecules that collectively increase expression and/or activity of the SCARB2 protein, (viii) one or more polynucleotides encoding the one or more RNAi molecules that collectively increase expression and/or activity of the SCARB2 protein, (ix) a SCARB2 protein, (x) one or more small molecules that collectively increase expression and/or activity of the SCARB2 protein.

E3. The method of E1 or E2, wherein the GBA is full-length GBA.

E4. The method of E1 or E2, wherein the GBA is a catalytic domain of GBA.

E5. The method of any one of E2-E4, wherein the GBA protein has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO. 1.

E6. The method of E5, wherein the GBA protein has an amino acid sequence that is at least 90% (e.g., at least 91%, 92%, 93%, 94%, 95%, 96%, 97%, 98%, 99%, or more) identical to the amino acid sequence of SEQ ID NO. 1.

E7. The method of E6, wherein the GBA has an amino acid sequence that is at least 95% (e.g., at least 96%, 97%, 98%, 99%, or more) identical to the amino acid sequence of SEQ ID NO. 1.

E8. The method of E7, wherein the GBA has the amino acid sequence of SEQ ID NO. 1.

E9. The method of any one of E2-E4, wherein the GBA has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO. 5.

E10. The method of E9, wherein the GBA has an amino acid sequence that is at least 90% (e.g., at least 91%, 92%, 93%, 94%, 95%, 96%, 97%, 98%, 99%, or more) identical to the amino acid sequence of SEQ ID NO. 5.

E11. The method of E10, wherein the GBA has an amino acid sequence that is at least 95% (e.g., at least 96%, 97%, 98%, 99%, or more) identical to the amino acid sequence of SEQ ID NO. 5.

E12. The method of E11, wherein the GBA has the amino acid sequence of SEQ ID NO. 5.

E13. The method of anyone of E1-E8, wherein the transgene encoding GBA has a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO. 6.

E14. The method of E13, wherein the transgene encoding GBA has a nucleic acid sequence that is at least 90% (e.g., at least 91%, 92%, 93%, 94%, 95%, 96%, 97%, 98%, 99%, or more) identical to the nucleic acid sequence of SEQ ID NO. 6.

E15. The method of E14, wherein the transgene encoding GBA has a nucleic acid sequence that is at least 95% (e.g., at least 96%, 97%, 98%, 99%, or more) identical to the nucleic acid sequence of SEQ ID NO. 6.

E16. The method of E15, wherein the transgene encoding GBA has the nucleic acid sequence of SEQ ID NO. 6.

E17. The method of any one of E1-E12, wherein the transgene encoding GBA has a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO. 7.

E18. The method of E17, wherein the transgene encoding GBA has a nucleic acid sequence that is at least 90% (e.g., at least 91%, 92%, 93%, 94%, 95%, 96%, 97%, 98%, 99%, or more) identical to the nucleic acid sequence of SEQ ID NO. 7.

E19. The method of E18, wherein the transgene encoding GBA has a nucleic acid sequence that is at least 95% (e.g., at least 96%, 97%, 98%, 99%, or more) identical to the nucleic acid sequence of SEQ ID NO. 7.

E20. The method of E19, wherein the transgene encoding GBA has the nucleic acid sequence of SEQ ID NO. 7.

E21. The method of any one of E1-E12, wherein the transgene encoding GBA has a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO. 11.

E22. The method of E21, wherein the transgene encoding GBA has a nucleic acid sequence that is at least 90% (e.g., at least 91%, 92%, 93%, 94%, 95%, 96%, 97%, 98%, 99%, or more) identical to the nucleic acid sequence of SEQ ID NO. 11.

E23. The method of E22, wherein the transgene encoding GBA has a nucleic acid sequence that is at least 95% (e.g., at least 96%, 97%, 98%, 99%, or more) identical to the nucleic acid sequence of SEQ ID NO. 11.

E24. The method of E23, wherein the transgene encoding GBA has the nucleic acid sequence of SEQ ID NO. 11.

E25. The method of any one of E1-E24, wherein the GBA comprises a signal peptide.

E26. The method of E25, wherein the signal peptide has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO. 12.

E27. The method of E26, wherein the signal peptide has an amino acid sequence that is at least 90% (e.g., at least 91%, 92%, 93%, 94%, 95%, 96%, 97%, 98%, 99%, or more) identical to the amino acid sequence of SEQ ID NO. 12.

E28. The method of E27, wherein the signal peptide has an amino acid sequence that is at least 95% (e.g., at least 96%, 97%, 98%, 99%, or more) identical to the amino acid sequence of SEQ ID NO. 12.

E29. The method of E28, wherein the signal peptide has the amino acid sequence of SEQ ID NO. 12.

E30. The method of any one of E26-E29, wherein the signal peptide is encoded by a polynucleotide having a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO. 13.

E31. The method of E30, wherein the signal peptide is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO. 13.

E32. The method of E31, wherein the signal peptide is encoded by a polynucleotide having a nucleic acid sequence that is at least 95% (e.g., at least 96%, 97%, 98%, 99%, or more) identical to the nucleic acid sequence of SEQ ID NO. 13.

E33. The method of E32, wherein the signal peptide is encoded by a polynucleotide having the nucleic acid sequence of SEQ ID NO. 13.

E34. The method of any one of E1-E33, wherein the transgene encoding GBA encodes non-secreted GBA.

E35. The method of E34, wherein the transgene encoding non-secreted GBA comprises a signal peptide.

E36. The method of E35, wherein the signal peptide is a GBA signal peptide.

E37. The method of any one of E1-E33, wherein the transgene encoding GBA encodes secreted GBA.

E38. The method of E37, wherein the transgene encoding secreted GBA comprises a secretory signal peptide.

E39. The method of E38, wherein the secretory signal peptide is an alpha-1 antitrypsin secretory signal peptide.

E40. The method of E38, wherein the secretory signal peptide is an insulin-like growth factor II (IGF-II) secretory signal peptide.

E41. The method of any one of E2-E40, wherein the transgene encoding GBA encodes a GBA fusion protein.

E42. The method of E41, wherein the GBA fusion protein has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO. 2.

E43. The method of E42, wherein the GBA fusion protein has an amino acid sequence that is at least 90% (e.g., at least 91%, 92%, 93%, 94%, 95%, 96%, 97%, 98%, 99%, or more) identical to the amino acid sequence of SEQ ID NO. 2.

E44. The method of E43, wherein the GBA fusion protein has an amino acid sequence that is at least 95% (e.g., at least 96%, 97%, 98%, 99%, or more) identical to the amino acid sequence of SEQ ID NO. 2.

E45. The method of E44, wherein the GBA fusion protein has the amino acid sequence of SEQ ID NO. 2.

E46. The method of E41, wherein the GBA fusion protein has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO. 3.

E47. The method of E46, wherein the GBA fusion protein has an amino acid sequence that is at least 90% (e.g., at least 91%, 92%, 93%, 94%, 95%, 96%, 97%, 98%, 99%, or more) identical to the amino acid sequence of SEQ ID NO. 3.

E48. The method of E47, wherein the GBA fusion protein has an amino acid sequence that is at least 95% (e.g., at least 96%, 97%, 98%, 99%, or more) identical to the amino acid sequence of SEQ ID NO. 3.

E49. The method of E48, wherein the GBA fusion protein has the amino acid sequence of SEQ ID NO. 3.

E50. The method of E41, wherein the GBA fusion protein has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO. 4.

E51. The method of E50, wherein the GBA fusion protein has an amino acid sequence that is at least 90% (e.g., at least 91%, 92%, 93%, 94%, 95%, 96%, 97%, 98%, 99%, or more) identical to the amino acid sequence of SEQ ID NO. 4.

E52. The method of E51, wherein the GBA fusion protein has an amino acid sequence that is at least 95% (e.g., at least 96%, 97%, 98%, 99%, or more) identical to the amino acid sequence of SEQ ID NO. 4.

E53. The method of E52, wherein the GBA fusion protein has the amino acid sequence of SEQ ID NO. 4.

E54. The method of E41, wherein the transgene encoding the GBA fusion protein has a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO. 8.

E55. The method of E54, wherein the transgene encoding the GBA fusion protein has a nucleic acid sequence that is at least 90% (e.g., at least 91%, 92%, 93%, 94%, 95%, 96%, 97%, 98%, 99%, or more) identical to the nucleic acid sequence of SEQ ID NO. 8.

E56. The method of E55, wherein the transgene encoding the GBA fusion protein has a nucleic acid sequence that is at least 95% (e.g., at least 96%, 97%, 98%, 99%, or more) identical to the nucleic acid sequence of SEQ ID NO. 8

E57. The method of E56, wherein the transgene encoding the GBA fusion protein has a nucleic acid sequence of SEQ ID NO. 8.

E58. The method of E41, wherein the transgene encoding the GBA fusion protein has a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO. 9.

E59. The method of E58, wherein the transgene encoding the GBA fusion protein has a nucleic acid sequence that is at least 90% (e.g., at least 91%, 92%, 93%, 94%, 95%, 96%, 97%, 98%, 99%, or more) identical to the nucleic acid sequence of SEQ ID NO. 9.

E60. The method of E59, wherein the transgene encoding the GBA fusion protein has a nucleic acid sequence that is at least 95% (e.g., at least 96%, 97%, 98%, 99%, or more) identical to the nucleic acid sequence of SEQ ID NO. 9.

E61. The method of E60, wherein the transgene encoding the GBA fusion protein has a nucleic acid sequence of SEQ ID NO. 9.

E62. The method of E41, wherein the transgene encoding the GBA fusion protein has a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO. 10.

E63. The method of E62, wherein the transgene encoding the GBA fusion protein has a nucleic acid sequence that is at least 90% (e.g., at least 91%, 92%, 93%, 94%, 95%, 96%, 97%, 98%, 99%, or more) identical to the nucleic acid sequence of SEQ ID NO. 10.

E64. The method of E63, wherein the transgene encoding the GBA fusion protein has a nucleic acid sequence that is at least 95% (e.g., at least 96%, 97%, 98%, 99%, or more) identical to the nucleic acid sequence of SEQ ID NO. 10.

E65. The method of E64, wherein the transgene encoding the GBA fusion protein has a nucleic acid sequence of SEQ ID NO. 10.

E66. The method of any one of E41-E65, wherein the GBA protein and/or the SCARB2 protein is a fusion protein comprising GBA or SCARB2 and a glycosylation independent lysosomal targeting (GILT) tag.

E67. The method of E66, wherein the GILT tag comprises a human IGF-II mutein having an amino acid sequence that is at least 70% (e.g., at least 71%, 75%, 80%, 85%, 90%, 91%, 92%, 93%, 94%, 95%, 96%, 97%, 98%, 99% or more) identical to the amino acid sequence of mature human IGF-II (SEQ ID NO. 22), and having diminished binding affinity for the insulin receptor relative to the affinity of naturally-occurring human IGF-II for the insulin receptor, wherein the IGF-II mutein is resistant to furin cleavage and binds to the human cation-independent mannose-6-phosphate receptor in a mannose-6-phosphate-independent manner.

E68. The method of E67, wherein the IGF-II mutein comprises a mutation within a region corresponding to amino acids 30-40 of SEQ ID NO. 22, and wherein the mutation abolishes at least one furin protease cleavage site.

E69. The method of E68, wherein the mutation is an amino acid substitution, deletion, and/or insertion.

E70. The method of E69, wherein the mutation is an Lys or Ala amino acid substitution at a position corresponding to Arg37 or Arg40 of SEQ ID NO. 22.

E71. The method of E70, wherein the mutation is a deletion or replacement of amino acid residues corresponding to positions selected form the group consisting of 31-40, 32-40, 33-40, 34-40, 30-39, 31-39, 32-39, 34-37, 33-39, 35-39, 36-39, 37-40, 34-40 of SEQ ID NO. 22, and combinations thereof.

E72. The method of any one of E66-E71, wherein the GILT tag has an amino acid sequence that is at least 70% (e.g., at least 71%, 75%, 80%, 85%, 90%, 91%, 92%, 93%, 94%, 95%, 96%, 97%, 98%, 99% or more) identical to the amino acid sequence of SEQ ID NO. 23.

E73. The method of E72, wherein the GILT tag has an amino acid sequence that is at least 80% identical to the amino acid sequence of SEQ ID NO. 23.

E74. The method of E73, wherein the GILT tag has an amino acid sequence that is at least 90% (e.g., at least 91%, 92%, 93%, 94%, 95%, 96%, 97%, 98%, 99%, or more) identical to the amino acid sequence of SEQ ID NO. 23.

E75. The method of E74, wherein the GILT tag has the amino acid sequence of SEQ ID NO. 23.

E76. The method of any one of E66-E71, wherein the GILT tag has an amino acid sequence that is at least 70% (e.g., at least 71%, 75%, 80%, 85%, 90%, 91%, 92%, 93%, 94%, 95%, 96%, 97%, 98%, 99% or more) identical to the amino acid sequence of SEQ ID NO. 24.

E77. The method of E76, wherein the GILT tag has an amino acid sequence that is at least 80% identical to the amino acid sequence of SEQ ID NO. 24.

E78. The method of E77, wherein the GILT tag has an amino acid sequence that is at least 90% (e.g., at least 91%, 92%, 93%, 94%, 95%, 96%, 97%, 98%, 99%, or more) identical to the amino acid sequence of SEQ ID NO. 24.

E79. The method of E78, wherein the GILT tag has the amino acid sequence of SEQ ID NO. 24.

E80. The method of any one of E66-E71, wherein the GILT tag has an amino acid sequence that is at least 70% (e.g., at least 71%, 75%, 80%, 85%, 90%, 91%, 92%, 93%, 94%, 95%, 96%, 97%, 98%, 99% or more) identical to the amino acid sequence of SEQ ID NO. 25.

E81. The method of E80, wherein the GILT tag has an amino acid sequence that is at least 80% identical to the amino acid sequence of SEQ ID NO. 25.

E82. The method of E81, wherein the GILT tag has an amino acid sequence that is at least 90% (e.g., at least 91%, 92%, 93%, 94%, 95%, 96%, 97%, 98%, 99%, or more) identical to the amino acid sequence of SEQ ID NO. 25.

E83. The method of E82, wherein the GILT tag has the amino acid sequence of SEQ ID NO. 25.

E84. The method of any one of E66-E83, wherein the GILT tag is encoded by a polynucleotide having a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO. 26.

E85. The method of E84, wherein the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO. 26.

E86. The method of E85, wherein the GILT tag is encoded by a polynucleotide having a nucleic acid sequence that is at least 95% (e.g., at least 96%, 97%, 98%, 99%, or more) identical to the nucleic acid sequence of SEQ ID NO. 26.

E87. The method of E86, wherein the GILT tag is encoded by a polynucleotide having the nucleic acid sequence of SEQ ID NO. 26.

E88. The method of any one of E66-E83, wherein the GILT tag is encoded by a polynucleotide having a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO. 27.

E89. The method of E88, wherein the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO. 27.

E90. The method of E89, wherein the GILT tag is encoded by a polynucleotide having a nucleic acid sequence that is at least 95% (e.g., at least 96%, 97%, 98%, 99%, or more) identical to the nucleic acid sequence of SEQ ID NO. 27.

E91. The method of E90, wherein the GILT tag is encoded by a polynucleotide having the nucleic acid sequence of SEQ ID NO. 27.

E92. The method of any one of E66-E83, wherein the GILT tag is encoded by a polynucleotide having a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO. 28.

E93. The method of E92, wherein the GILT tag is encoded by a polynucleotide having a nucleic acid sequence that is at least 90% (e.g., at least 91%, 92%, 93%, 94%, 95%, 96%, 97%, 98%, 99%, or more) identical to the nucleic acid sequence of SEQ ID NO. 28.

E94. The method of E93, wherein the GILT tag is encoded by a polynucleotide having a nucleic acid sequence that is at least 95% (e.g., at least 96%, 97%, 98%, 99%, or more) identical to the nucleic acid sequence of SEQ ID NO. 28.

E95. The method of E94, wherein the GILT tag is encoded by a polynucleotide having the nucleic acid sequence of SEQ ID NO. 28.

E96. The method of any one of E41-E95, wherein the GBA fusion protein and/or the SCARB2 fusion protein comprises a receptor-binding (Rb) domain of apolipoprotein E (ApoE).

E97. The method of E96, wherein the Rb domain comprises a portion of ApoE having the amino acid sequence of residues 25-185, 50-180, 75-175, 100-170, 125-160, or 130-150 of SEQ ID NO. 29.

E98. The method of E97, wherein the Rb domain comprises a region having at least 70% (e.g., at least 71%, 75%, 80%, 85%, 90%, 91%, 92%, 93%, 94%, 95%, 96%, 97%, 98%, 99% or more) sequence identity to the amino acid sequence of residues 159-167 of SEQ ID NO. 29.

E99. The method of any one of E1-E98, wherein the SCARB2 has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO: 14.

E100. The method of E99, wherein the SCARB2 has an amino acid sequence that is at least 90% (e.g., at least 91%, 92%, 93%, 94%, 95%, 96%, 97%, 98%, 99%, or more) identical to the amino acid sequence of SEQ ID NO: 14.

E101. The method of E100, wherein the SCARB2 has an amino acid sequence that is at least 95% (e.g., at least 96%, 97%, 98%, 99%, or more) identical to the amino acid sequence of SEQ ID NO: 14.

E102. The method of E101, wherein the SCARB2 has an amino acid sequence of SEQ ID NO: 14.

E103. The method of any one of E1-E98, wherein the SCARB2 has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO: 15.

E104. The method of E103, wherein the SCARB2 has an amino acid sequence that is at least 90% (e.g., at least 91%, 92%, 93%, 94%, 95%, 96%, 97%, 98%, 99%, or more) identical to the amino acid sequence of SEQ ID NO: 15.

E105. The method of E104, wherein the SCARB2 has an amino acid sequence that is at least 95% (e.g., at least 96%, 97%, 98%, 99%, or more) identical to the amino acid sequence of SEQ ID NO: 15.

E106. The method of E105, wherein the SCARB2 has an amino acid sequence of SEQ ID NO: 15.

E107. The method of any one of E1-E98, wherein the polynucleotide encoding SCARB2 has a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO: 16. E108. The method of E107, wherein the polynucleotide encoding SCARB2 has a nucleic acid sequence that is at least 90% (e.g., at least 91%, 92%, 93%, 94%, 95%, 96%, 97%, 98%, 99%, or more) identical to the nucleic acid sequence of SEQ ID NO: 16.

E109. The method of E108, wherein the polynucleotide encoding SCARB2 has a nucleic acid sequence that is at least 95% (e.g., at least 96%, 97%, 98%, 99%, or more) identical to the nucleic acid sequence of SEQ ID NO: 16.

E110. The method of E109, wherein the polynucleotide encoding SCARB2 has a nucleic acid sequence of SEQ ID NO: 16.

E111. The method of any one of E1-E98, wherein the polynucleotide encoding SCARB2 has a nucleic acid sequence that is at least 85% (e.g., at least 86%, 87%, 88%, 89%, 90%, 91%, 92%, 93%, 94%, 95%, 96%, 97%, 98%, 99%, or more) identical to the nucleic acid sequence of SEQ ID NO: 17.

E112. The method of E111, wherein the polynucleotide encoding SCARB2 has a nucleic acid sequence that is at least 90% (e.g., at least 91%, 92%, 93%, 94%, 95%, 96%, 97%, 98%, 99%, or more) identical to the nucleic acid sequence of SEQ ID NO: 17.

E113. The method of E112, wherein the polynucleotide encoding SCARB2 has a nucleic acid sequence that is at least 95% (e.g., at least 96%, 97%, 98%, 99%, or more) identical to the nucleic acid sequence of SEQ ID NO: 17.

E114. The method of E113, wherein the polynucleotide encoding SCARB2 has a nucleic acid sequence of SEQ ID NO: 17.

E115. The method of any one of E1-E114, wherein the transgene encoding GBA and/or SCARB2 further comprises a microRNA (miRNA) targeting sequence in the 3′-UTR.

E116. The method of E115, wherein the miRNA targeting sequence is a miR-126 targeting sequence.

E117. The method of any one of E34-E116, wherein the GBA and/or SCARB2 penetrates the BBB in the subject.

E118. The method of E2, wherein the one or more RNAi molecules comprise short interfering RNA (siRNA), short hairpin RNA (shRNA), and/or microRNA (miRNA).

E119. The method of any one of E2-E118, wherein the one or more nucleic acid molecules are provided to the subject by administering to the subject a composition comprising a population of cells that together contain nucleic acids encoding the GBA and/or SCARB2 protein.

E120. The method of E119, wherein the population is a uniform population of cells that contain nucleic acids encoding the proteins or a heterogeneous population of cells that together contain nucleic acids encoding the GBA and/or SCARB2 protein.

E121. The method of E119 or E120, wherein the cells are pluripotent cells or multipotent cells.

E122. The method of E121, wherein the multipotent cells are CD34+ cells.

E123. The method of E122, wherein the CD34+ cells are HSCs or MPCs.

E124. The method of E121, wherein the pluripotent cells are ESCs or iPSCs.

E125. The method of E119 or 120, wherein the cells are BLPCs, microglial progenitor cells, monocytes, macrophages, or microglia.

E126. The method of E125, wherein the BLPCs are monocytes.

E127. The method of any one of E1-E126, wherein a population of endogenous hematopoietic cells in the subject has been ablated prior to administration of the one or more agents to the subject, optionally wherein the hematopoietic cells are CD34+ cells, BLPCs, microglial progenitor cells, monocytes, macrophages, or microglia.

E128. The method of any one of E1-E126, the method comprising ablating a population of endogenous hematopoietic cells in the subject prior to administering the one or more agents to the subject, optionally wherein the hematopoietic cells are CD34+ cells, BLPCs, microglial progenitor cells, monocytes, macrophages, or microglia.

E129. The method of E127 or E128, wherein the microglia are ablated using an agent selected from the group consisting of busulfan, PLX3397, PLX647, PLX5622, treosulfan, and clodronate liposomes, by radiation therapy, or a combination thereof.

E130. The method of any one of E1-E129, wherein the one or more agents is administered systemically to the subject.

E131. The method of E130, wherein the one or more agents is administered to the subject by way of intravenous injection.

E132. The method of any one of E1-E129, wherein the one or more agents is administered directly to the central nervous system of the subject.

E133. The method of E132, wherein the one or more agents is administered to the subject by way of intracerebroventricular injection, stereotactic injection, or a combination thereof.

E134. The method of any one of E1-E129, wherein the one or more agents is administered directly to the bone marrow of the subject.

E135. The method of E134, wherein the one or more agents is administered to the subject by way of intraosseous injection.

E136. The method of any one of E1-E129, wherein the one or more agents is administered to the subject by way of a bone marrow transplant comprising the one or more agents.

E137. The method of any one of E1-E129, wherein the one or more agents is administered to the subject by way of intracerebroventricular injection.

E138. The method of any one of E1-E129, wherein the one or more agents is administered to the subject by way of intravenous injection.

E139. The method of any one of E1-E129, wherein the one or more agents is administered to the subject by direct administration to the central nervous system of the subject and by systemic administration.

E140. The method of E139, wherein the one or more agents is administered to the subject by way of intracerebroventricular injection and intravenous injection.

E141. The method of any one of E119-E140, wherein the cells are autologous cells or allogeneic cells.

E142. The method of any one of E119-E141, wherein the cells are transfected or transduced ex vivo to express the GBA and/or SCARB2.

E143. The method of E142, wherein the cells are transduced with a viral vector selected from the group consisting of an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus.

E144. The method of E143, wherein the viral vector is a Retroviridae family viral vector.

E145. The method of E144, wherein the Retroviridae family viral vector is a lentiviral vector.

E146. The method of E144, wherein the Retroviridae family viral vector is an alpharetroviral vector.

E147. The method of E144, wherein the Retroviridae family viral vector is a gammaretroviral vector.

E148. The method of any one of E143-E147, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

E149. The method of E143, wherein the viral vector is an AAV selected from the group consisting of AAV1, AAV2, AAV3, AAV4, AAV5, AAV6, AAV7, AAV8, AAV9, AAV10, and AAVrh74.

E150. The method of E143, wherein the viral vector is a pseudotyped viral vector.

E151. The method of E150, wherein the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

E152. The method of any one of E121-E152, wherein the pluripotent cells are transduced to express the GBA and SCARB2 from separate monocistronic expression cassettes.

E153. The method of any one of E121-E152, wherein the pluripotent cells are transduced to express the GBA and SCARB2 from a polycistronic expression cassette.

E154. The method of E153, wherein the pluripotent cells are transduced to express the GBA and SCARB2 from a bicistronic expression cassette.

E155. The method of E153 or E154, wherein the polycistronic expression cassette comprises an internal ribosomal entry site (IRES) positioned between a polynucleotide encoding the GBA and a polynucleotide encoding the SCARB2.

E156. The method of E155, wherein the polycistronic expression cassette comprises 2A polynucleotide (e.g., F2A, P2A, E2A, or T2A polynucleotide) positioned between a polynucleotide encoding the GBA and a polynucleotide encoding the SCARB2.

E157. The method of E142, wherein the cells are transfected using: a) an agent selected from the group consisting of a cationic polymer, diethylaminoethyldextran, polyethylenimine, a cationic lipid, a liposome, calcium phosphate, an activated dendrimer, and a magnetic bead; or b) a technique selected from the group consisting of electroporation, Nucleofection, squeeze-poration, sonoporation, optical transfection, Magnetofection, and impalefection.

E158. The method of any one of E142-E157, wherein the pluripotent cells are transfected ex vivo to express the GBA or SCARB2.

E159. The method of E121-E152, wherein the pluripotent cells are transfected ex vivo to express the GBA and the SCARB2 from separate, monocistronic expression cassettes.

E160. The method of E159, wherein the monocistronic expression cassettes are located within two or more separate plasmids.

E161. The method of E159, wherein the monocistronic expression cassettes are located on a single plasmid.

E162. The method of E121-E152, wherein the pluripotent cells are transfected ex vivo to express the GBA and the SCARB2 from a polycistronic expression cassette.

E163. The method of E162, wherein the pluripotent cells are transfected ex vivo to express the GBA and SCARB2 from a bicistronic expression cassette.

E164. The method of E162 or E163, wherein the polycistronic expression cassette comprises an IRES positioned between a polynucleotide encoding the GBA protein and a polynucleotide encoding the SCARB2 protein.

E165. The method of any one of E162-E164, wherein the polycistronic expression cassette comprises an 2A polynucleotide positioned between a polynucleotide encoding the GBA protein and a polynucleotide encoding the SCARB2 protein.

E166. The method of E165, wherein the 2A sequence is selected from the group consisting of F2A, P2A, E2A, and T2A.

E167. The method of any one of E2-E166, wherein the one or more nucleic acid molecules are provided to the subject by administering to the subject one or more viral vectors that together comprise the one or more nucleic acid molecules.

E168. The method of E167, wherein the subject is administered a plurality of viral vectors that together comprise the one or more nucleic acid molecules.

E169. The method of E167, wherein the subject is administered a plurality of viral vectors that each individually comprise the one or more nucleic acid molecules.

E170. The method of any one of E167-E169, wherein the viral vector is a Retroviridae family viral vector.

E171. The method of E170, wherein the Retroviridae family viral vector is a lentiviral vector, alpharetroviral vector, or gamma retroviral vector.

E172. The method of any one of E170 or E171, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

E173. The method of any one of E167-E169, wherein the viral vector is an AAV selected from the group consisting of AAV1, AAV2, AAV3, AAV4, AAV5, AAV6, AAV7, AAV8, AAV9, AAV10, and AAVrh74.

E174. The method of any one of E167-E169, wherein the viral vector is a pseudotyped viral vector.

E175. The method of E174, wherein the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

E176. The method of any one of E2-E175, wherein one or more of the nucleic acid molecules comprises a transgene encoding one or more of the proteins operably linked to a ubiquitous promoter, a cell lineage-specific promoter, or a synthetic promoter.

E177. The method of E176, wherein the ubiquitous promoter is selected from the group consisting of an elongation factor 1-alpha (EF1α) promoter, phosphoglycerate kinase 1 (PGK) promoter, or EF1α promoter containing elements of locus control region of the β-globin gene containing regions of erythroid-specific DNase I hypersensitivity (HS) regions 2, 3, and 4 (β-LCR(HS4,3,2)-EFS promoter).

E178. The method of E176, wherein the cell lineage-specific promoter is selected from the group consisting of a CD68 molecule (CD68) promoter, CD11 b molecule (CD11b) promoter, C-X3-C motif chemokine receptor 1 (CX3CR1) promoter, allograft inflammatory factor 1 promoter (AIF1) promoter, purinergic receptor P2Y12 (P2Y12) promoter, transmembrane protein 119 (TMEM119) promoter, or colony stimulating factor 1 receptor (CSF1R) promoter.

E179. The method of E176, wherein the synthetic promoter is a Myeloproliferative Sarcoma Virus Enhancer, Negative Control Region Deleted, dl587rev Primer-Binding Site Substituted (MND) promoter.

E180. The method of E179, wherein the MND promoter comprises a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 20.

E181. The method of E179, wherein the MND promoter comprises a polynucleotide having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 21.

E182. The method of any one of E2-E181, wherein the one or more nucleic acid molecules comprise a microRNA (miRNA)-126 (miR-126) targeting sequence in the 3′-UTR.

E183. The method of any one of E2-E182, wherein upon providing the one or more nucleic acid molecules to the subject, the proteins penetrate the blood-brain barrier in the subject.

E184. The method of any one of E2-E183, wherein, prior to providing the subject with the one or more nucleic acid molecules, endogenous expression of the GBA protein is disrupted in the cells, in the subject, or in a population of neurons in the subject.

E185. The method of E184, wherein the endogenous expression is disrupted by contacting the cells with a nuclease that catalyzes cleavage of an endogenous gene encoding one of the proteins.

E186. The method of E185, wherein the nuclease is a CRISPR associated protein 9 (Cas9), CRISPR-associated protein 12a (Cas12a), a transcription activator-like effector nuclease, a meganuclease, or a zinc finger nuclease.

E187. The method of E184, wherein endogenous expression of GBA is disrupted by administering an inhibitory RNA molecule to the cells, the subject, or the population of neurons.

E188. The method of E187, wherein the inhibitory RNA molecule is a siRNA, a shRNA, or a miRNA.

E189. The method of any one of E1-E188, wherein the Gaucher's disease is associated with one or more mutations in the GBA gene.

E190. The method of E189, wherein the one or more mutations in the GBA gene comprise a p.N370S substitution, p.R463C substitution, p.L444P substitution, p.D409H substitution, p.R463C substitution, p.R496H substitution, p. F252I substitution, p.A456P substitution, p.V460V substitution, p.V394L, p.E326K substitution, p.G377S substitution, p.N188S substitution, c.84insG insertion, c.84dupG (84GG) duplication, c.115+1G>A substitution, or c.IVS2DS+1G-A splice site mutation.

E191. The method of any one of E1-E190, wherein the Gaucher disease is Type 1 Gaucher disease.

E192. The method of E191, wherein the subject has a confirmed diagnosis of Type 1 Gaucher disease based on genotyping, deficient GBA activity in the blood of the subject, and/or clinical phenotype.

E193. The method of E192, wherein the deficient GBA activity in the subject is defined as activity that is equal to or greater than 15% of activity of GBA in a control reference patient not diagnosed as having Gaucher disease.

E194. The method of any one of E1-E190, wherein the Gaucher disease is Type 2 Gaucher disease.

E195. The method of any one of E1-E190, wherein the Gaucher disease is Type 3 Gaucher disease.

E196. The method of any one of E1-E195, wherein the Gaucher's disease is associated with one or more mutations in the SCARB2 gene.

E197. The method of E196, wherein the one or more mutations in the SCARB2 gene comprise a p.Q471 G substitution, p.H363N substitution, p.Q288Ter nonsense mutation, p.W178Ter nonsense mutation, p.W146fs frameshift mutation, p.Glu420fs frameshift mutation, g.76168478T>G transversion mutation, g.1239+1G-T splice site mutation, or g.76168401dup splice site mutation.

E198. The method of any one of E1-E197, wherein the subject is a human.

E199. The method of any one of E1-E198, wherein the subject has undergone enzyme replacement therapy (ERT) comprising a total monthly dose of GBA ERT that is greater than 30 U/kg and less than 120 U/kg for 24 or more consecutive months at a time of treatment with the one or more agents.

E200. The method of E199, wherein the subject has received a biweekly dose of GBA ERT greater than or equal to 15 U/kg and less than or equal to 60 U/kg.

E201. The method of E200, wherein the subject has received a weekly dose of GBA ERT greater than or equal to 7.5 U/kg and less than or equal to 30 U/kg.

E202. The method of E1-E201, wherein the subject has received substrate reduction therapy (SRT) for Gaucher disease during the 24 months immediately preceding treatment with the one or more agents.

E203. The method of E1-E202, wherein the subject has not received SRT for Gaucher disease during the 24 months immediately preceding treatment with the one or more agents.

E204. The method of any one of E2-E203, wherein the SCARB2 comprises a signal peptide, wherein the signal peptide has an amino acid sequence that is at least 70% identical to the amino acid sequence of SEQ ID NO: 49.

E205. The method of any one of E2-204, wherein the signal peptide has an amino acid sequence that is at least 75% (e.g., at least 76%, 80%, 85%, 90%, 95%, 96%, 97%, 98%, 99%, or more) identical to the amino acid sequence of SEQ ID NO: 49.

E206. The method of E205, wherein the signal peptide has an amino acid sequence that is at least 80% (e.g., at least 81%, 85%, 90%, 95%, 96%, 97%, 98%, 99%, or more) identical to the amino acid sequence of SEQ ID NO: 49.

E207. The method of E206, wherein the signal peptide has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO: 49.

E208. The method of E207, the signal peptide has an amino acid sequence that is at least 90% (e.g., at least 91%, 92%, 93%, 94%, 95%, 96%, 97%, 98%, 99%, or more) identical to the amino acid sequence of SEQ ID NO:49.

E209. The method of E208, wherein the signal peptide has an amino acid sequence that is at least 95% (e.g., at least 96%, 97%, 98%, 99%, or more) identical to the amino acid sequence of SEQ ID NO: 49.

E210. The method of E209, wherein the signal peptide has an amino acid sequence of SEQ ID NO: 49.

E211. The method of any one of E2-E210, wherein the SCARB2 is a GBA-binding domain of SCARB2.

E212. The method of E211, wherein the GBA-binding domain of the SCARB2 protein has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO: 18.

E213. The method of E212, wherein the GBA-binding domain of the SCARB2 protein has an amino acid sequence that is at least 90% (e.g., at least 91%, 92%, 93%, 94%, 95%, 96%, 97%, 98%, 99%, or more) identical to the amino acid sequence of SEQ ID NO: 18.

E214. The method of E213, wherein the GBA-binding domain of the SCARB2 protein has an amino acid sequence that is at least 95% (e.g., at least 96%, 97%, 98%, 99%, or more) identical to the amino acid sequence of SEQ ID NO: 18.

E215. The method of E214, wherein the GBA-binding domain of the SCARB2 protein has an amino acid sequence of SEQ ID NO: 18.

E216. The method of E211, the GBA-binding domain of the SCARB2 protein has an amino acid sequence that is at least 85% (e.g., at least 86%, 87%, 88%, 89%, 90%, 91%, 92%, 93%, 94%, 95%, 96%, 97%, 98%, 99%, or more) identical to the amino acid sequence of SEQ ID NO: 19.

E217. The method of E216, wherein the GBA-binding domain of the SCARB2 protein has an amino acid sequence that is at least 90% (e.g., at least 91%, 92%, 93%, 94%, 95%, 96%, 97%, 98%, 99%, or more) identical to the amino acid sequence of SEQ ID NO: 19.

E218. The method of E217, wherein the GBA-binding domain of the SCARB2 protein has an amino acid sequence that is at least 95% (e.g., at least 96%, 97%, 98%, 99%, or more) identical to the amino acid sequence of SEQ ID NO: 19.

E219. The method of E218, wherein the GBA-binding domain of the SCARB2 protein has an amino acid sequence of SEQ ID NO: 19.

E220. The method of any one of E2-E219, wherein the GBA protein or the SCARB2 protein is a fusion protein comprising GBA or SCARB2 and a cell-penetrating peptide (CPP).

E221. The method of E220, wherein the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of any one of SEQ ID NOs: 30-48.

E222. The method of E221, wherein the CPP has an amino acid sequence having at least 90% (e.g., at least 91%, 92%, 93%, 94%, 95%, 96%, 97%, 98%, 99%, or more) sequence identity to the amino acid sequence of any one of SEQ ID NOs: 30-48.

E223. The method of E222, wherein the CPP has an amino acid sequence having at least 95% (e.g., at least 96%, 97%, 98%, 99%, or more) sequence identity to the amino acid sequence of any one of SEQ ID NOs: 30-48.

E224. The method of E223, wherein the CPP has an amino acid sequence having the amino acid sequence of any one of SEQ ID NOs: 30-48.

### BRIEF DESCRIPTION OF THE DRAWINGS

FIG. 1A is a diagram of an exemplary GBA transgene construct containing, in the N-terminal to C-terminal direction, a polypeptide sequence encoding a GBA signal peptide (SP) and a GBA catalytic domain that has been codon optimized for expression in human cells (GBA-co CD).

FIG. 1B is a diagram of an exemplary GBA transgene construct containing, in the N-terminal to C-terminal direction, a polypeptide sequence encoding a GBA SP, an Rb domain of ApoE (Rb-ApoE), and codon-optimized GBA (GBA-co).

FIG. 1C is a diagram of an exemplary GBA transgene construct containing, in the N-terminal to C-terminal direction, a polypeptide sequence encoding a codon-optimized human insulin-like growth factor II secretory signal peptide (IGF-II-co SSP), a GILT-tag (GILT) containing an R37A mutation, a linker (L), and GBA-co.

FIG. 1D is a diagram of an exemplary GBA transgene construct containing, in the N-terminal to C-terminal direction, a polypeptide sequence encoding an IGF-II-co SSP, a first linker (L), Rb-ApoE, a second linker, GILT containing an R37A mutation, a third linker, and GBA-co.

FIG. 1E is a diagram of an exemplary GBA transgene construct containing, in the N-terminal to C-terminal direction, a polypeptide sequence encoding an IGF-II-co SSP, a first linker (L), GILT containing an R37A mutation, a second linker (L), Rb-ApoE, a third linker (L), and GBA-co containing a microRNA targeting sequence (miRT) in the three prime untranslated region (3′-UTR).

FIG. 1F is a diagram of an exemplary GBA transgene construct containing, in the N-terminal to C-terminal direction, a polypeptide sequence encoding an IGF-II-co SSP, a first linker (L), GBA-co, a second linker, and GILT containing a R37A mutation.

FIG. 1G is a diagram of an exemplary GBA transgene construct containing, in the N-terminal to C-terminal direction, a polypeptide sequence encoding an IGF-II-co SSP, a first linker (L), GBA-co, a second linker, and Rb-ApoE.

FIGS. 2A-2D are a series of bar plots demonstrating GBA enzymatic activity and protein levels in mammalian cell lines transduced with either green fluorescent protein (GFP) or GBA constructs. FIG. 2A and FIG. 2C show GBA enzymatic activity measured from cell lysates of human HEK293T cells and murine RAW264.7 cells, respectively, following lentiviral transduction with GFP (black bars) or codon-optimized GBA (GBAco) constructs (grey bars). GBA enzymatic activity was measured in using a 4-methylumbelliferyl β-D-glucopyranoside (4MUG) substrate, which is enzymatically converted by GBA to produce a fluorescent product, 4-Methylumbelliferone (4MU). Tested GBAco constructs included: 1) GBAco alone; 2) GBAco, a C-terminal glycosylation independent lysosomal targeting (GILT) tag, and a peptide linker; 3) GBAco and a modified signal peptide sequence; 4) GBAco, a GILT tag, and a rigid peptide linker; or 5) GBAco, a GILT tag, and an XTEN linker. All lentivirally-encoded GBAco constructs resulted in increased GBA enzymatic activity in both of the tested cell lines. FIG. 2B and FIG. 2D show GBA protein levels as assayed by Western blot analysis of cell lysates from HEK293T and RAW264.7 cells, respectively, following lentiviral transduction with GFP (black bars) or codon-optimized GBA (GBAco) constructs (grey bars). All lentivirally-encoded GBAco constructs resulted in increased GBA protein levels in both of the tested cell lines. Data are represented as mean±SEM; n=3 independent transductions. Asterisks signify p-values less than 0.05, as determined using one-way ANOVA tests for statistical significance.

FIG. 3 is a Western blot analysis of glycosylated, transgene-derived GBA taken from cell lysates of HEK293T cells. Cells were transduced with one of three lentivirally-encoded GBAco constructs selected from the group including: 1) GBAco alone; 2) GBAco, a GILT tag, and a rigid peptide linker; or 3) GBAco, a GILT tag, and an XTEN linker. Cell lysates were treated with either EndoH or PNGase F glycosidases to assess changes in N-linked glycosylation through increased electrophoretic mobility. Western blot analysis detected engineered GBAco proteins at the predicted molecular weights, suggesting that GILT and linker peptides were stably expressed. Furthermore, electrophoretic mobility of engineered GBA proteins increased after enzymatic de-glycosylation, demonstrating that these proteins undergo physiological glycosylation.

FIGS. 4A-4B are a series of bar plots demonstrating GBA enzymatic activity in murine lineage-negative (Lin⁻) cells from wildtype and GBA mutant mice. Lin⁻ cells were isolated from the bone marrow and transduced with lentiviral vectors encoding GFP or GBAco. FIG. 4A shows a bar plot of GBA enzymatic activity in Lin⁻ cell lysate from wildtype and GBA-deficient transgenic mice (Gba^{D409V/+} Gba^{D409V/+}; Thy1-SNCA and Gba^{D409V/D409V}) transduced with a vector encoding GFP (black bars) or GBAco (grey bars). Enzymatic assays of Lin⁻ cells demonstrated that the heterozygous and homozygous Gba mutations reduced GBA activity by 43% and 92%, respectively, in the absence of GBA transgenes (WT: 13.04±0.644 nmol hr⁻¹ mg⁻¹; Gba^{D409V/+}: 7.49±0.293 nmol hr⁻¹ mg⁻¹; Thy1-SNCA; Gba^{D409V/+}: 7.14±0.252 nmol hr⁻¹ mg⁻¹; Thy1-SNCA; Gba^{D409V/D409V}: 1.20±0.114 nmol hr⁻¹ mg⁻¹; p<0.001, ANOVA with Tukey post-hoc analysis, FIG. 4A). Lentiviral transduction of Lin⁻ cells with a GBAco construct substantially increased GBA activity across all tested murine lines (compared to GFP control; p<0.001, ANOVA with Tukey post-hoc analysis). FIG. 4B shows a bar plot of GBA activity in Lin⁻ cell conditioned medium from Gba^{D409V/+}, Gba^{D409V/+}; Thy1-SNCA and Gba^{D409V/D409V} mice (total n=8 animals) after lentiviral transduction with GFP (black bars) or GBAco (grey bars), demonstrating increased detection of GBA activity. Data are represented as mean±SEM (n=1 independent transduction). Combined, these findings demonstrate that lentiviral GBAco constructs produce a functional GBA enzyme in hematopoietic stem cells (e.g., murine Lin⁻ cells) and can rescue GBA activity and expression levels in murine models of GBA deficiency.

FIG. 5 is a bar graph showing GBA (“Gcase”) activity in murine RAW264.7 macrophages transduced with lentiviral vectors encoding GBA and SCARB2. RAW264.7 cells were transduced with biscistronic lentiviral vectors encoding GBA and SCARB2 proteins separated by either a P2A, E2A, or T2A sequence. GBA activity was substantially increased in cells receiving both GBA and SCARB2 proteins, as compared to GBA alone.

FIGS. 6A-6C are bar graphs showing GBA (“GCase”) activity in Lin− hematopoietic stem/progenitor cells. Gba mutant donor mice were sacrificed, bone marrow cells were harvested, and lineage negative (Lin−) hematopoietic stem/progenitor cells were isolated. Lin− cells were then transduced with a lentiviral vector encoding GFP, human GBA, human SCARB2, human GBA fused with the GILT tag, or human GBA and human SCARB2 (separated by a P2A sequence). These cells were collected and washed approximately 16 hours later. Cells were analyzed for vector copy number per genome by qPCR (FIG. 6A), pluripotency (FIG. 6B), and GCase enzymatic activity by 4-MU assay (FIG. 6C).

FIGS. 7A-7C are bar graphs showing GBA (“GCase”) activity in bone marrow cells. Following collection and washing of cells as in FIGS. 6A-6C, the cells were transplanted into busulfan-conditioned Gba mutant mice (Gba D409V knock-in or KI). Approximately 8 weeks later, mice were sacrificed, and bone marrow were collected and analyzed for vector copy number per genome by qPCR (FIG. 7A), GCase enzymatic activity by 4-MU assay (FIG. 7B), and substrate levels by mass spectrometry (FIG. 7C).

FIGS. 8A-8C are bar graphs showing GBA (“GCase”) activity in the lung. Lungs were collected and analyzed for vector copy number per genome by qPCR (FIG. 8A), GCase enzymatic activity by 4-MU assay (FIG. 8B), and substrate levels by mass spectrometry (FIG. 8C).

### DEFINITIONS

As used herein, “administration” refers to providing or giving a subject a therapeutic agent (e.g., one or more agents that collectively increase expression of glucocerebrosidase (GBA) and scavenger receptor class B member 2 (SCARB2) proteins) by any effective route. Exemplary routes of administration are described herein below (e.g., intracerebroventricular (ICV) injection, intravenous (IV) injection, and stereotactic injection).

As used herein, “allogeneic” means cells, tissue, DNA, or factors taken or derived from a different subject of the same species. For example, in the context where allogeneic transduced pluripotent cells are administered to a subject with Gaucher disease, pluripotent cells derived from cells obtained from a patient that is not the subject are transduced or transfected with a vector that directs the expression of GBA and SCARB2, and the transduced cells are administered to the subject. The phrase “directs expression” refers to the polynucleotide containing a sequence that encodes the molecule to be expressed. The polynucleotide may contain additional sequence that enhances expression of the molecule in question.

As used herein, “autologous” refers to cells, tissue, DNA, or factors taken or derived from an individual's own tissues, cells, or DNA. For example, in the context where autologous transduced pluripotent cells are administered to a subject with Gaucher disease, pluripotent cells derived from cells obtained from the subject are transduced or transfected with a vector that directs the expression of GBA and SCARB2, and the transduced cells are administered to the subject.

As used herein, the term “ApoE” refers to apolipoprotein E, a member of a class of proteins involved in lipid transport. Apolipoprotein E is a fat-binding protein (apolipoprotein) that is part of the chylomicron and intermediate-density lipoprotein (IDLs). These are essential for the normal processing (catabolism) of triglyceride-rich lipoproteins. ApoE is encoded by the APOE gene. The term “ApoE” also refers to variants of the wild-type ApoE protein, such as proteins having at least 85% identity (e.g., 85%, 86% e.g., 85%, 86%, 87%, 88%, 89%, 90%, 91%, 92%, 93%, 94%, 95%, 96%, 97%, 98%, 99%, or 99.9% identity, or more) to the amino acid sequence of wild-type ApoE, which is set forth in SEQ ID NO: 21.

As used herein, the term “blood lineage progenitor cell” or “BLPC” refers to any cell (e.g., a mammalian cell) capable of differentiating into one or more (e.g., 2, 3, 4, 5 or more) types of hematopoietic (i.e., blood) cells. A BLPC may differentiate into erythrocytes, leukocytes (e.g., such as granulocytes (e.g., basophils, eosinophils, neutrophils, and mast cells) or a granulocytes (e.g., lymphocytes and monocytes)), or thrombocytes. A BLPC may also include a differentiated blood cell (e.g., a monocyte) that can further differentiate into another blood cell type (e.g., a macrophage).

As used herein, the terms “cell-penetrating peptide” and “CPP” refer to a polypeptide that is capable of crossing a cell membrane (e.g., a mammalian cell membrane) and entering the intracellular environment. A cell-penetrating peptide may cross or penetrate a cell membrane by any of a variety of mechanisms, including via endocytosis, macropinocytosis, and passive diffusion through membrane pores, among others. A cell-penetrating peptide may be capable of translocating a molecule to which it is chemically bound (e.g., a polypeptide bound by a covalent bond to the cell-penetrating peptide, such as, e.g., a GBA or a SCARB2 polypeptide) across a cell membrane. Cell-penetrating peptides include those that enter the cell via endocytosis and reside within endocytic vesicles. In certain cases, as an endocytic vesicle matures, a cell-penetrating peptide may enter the cytosol of a cell. Under other conditions, a vesicle containing a cell-penetrating peptide may fuse to another organelle within a cell, releasing the contents of the vesicle into the organelle. Exemplary CPPs are provided in SEQ ID NOs: 30-48.

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, “codon optimization” refers a process of modifying a nucleic acid sequence in accordance with the principle that the frequency of occurrence of synonymous codons (e.g., codons that code for the same amino acid) in coding DNA is biased in different species. Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. Codon optimization may be performed in a manner such as that described in, e.g., U.S. Pat. Nos. 7,561,972, 7,561,973, and 7,888,112, each of which is incorporated herein by reference in its entirety. The sequence surrounding the translational start site can be converted to a consensus Kozak sequence according to known methods. See, e.g., Kozak et al, Nucleic Acids Res. 15 (20): 8125-8148, incorporated herein by reference in its entirety. Multiple stop codons can be incorporated.

As used herein, the term “cistron” refers to a segment of a DNA or RNA sequence encoding a single protein or polypeptide product.

As used herein, the terms “condition” and “conditioning” refer to processes by which a subject is prepared for receipt of a transplant containing pluripotent stem cells (e.g., CD34+ cells). Such procedures promote the engraftment of a pluripotent stem cell transplant, for example, by selectively depleting endogenous microglia or hematopoietic stem cells, thereby creating a vacancy filled by an exogenous pluripotent stem cell transplant. According to the methods described herein, a subject may be conditioned for pluripotent stem cell transplant therapy by administration to the subject of one or more agents capable of ablating endogenous microglia and/or hematopoietic stem or progenitor cells (e.g., busulfan, treosulfan, PLX3397, PLX647, PLX5622, and clodronate liposomes), radiation therapy, or a combination thereof. Conditioning may be myeloablative or non-myeloablative. Other cell-ablating agents and methods well known in the art (e.g., antibody-drug conjugates) may also be used.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in table 1 below.

TABLE 1
Representative physicochemical properties of naturally-occurring amino acids

| | | | | Electrostatic | |
|---|---|---|---|---|---|
| | | | Side- | character at | |
| | 3 Letter | 1 Letter | chain | physiological pH | Steric |
| Amino Acid | Code | Code | Polarity | (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term “disrupt”, with respect to a gene, refers to preventing the formation of a functional gene product. A gene product is functional if it fulfills its normal (wild-type) functions.

Disruption of the gene prevents expression of a functional factor encoded by the gene and contains an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods for genetically modifying pluripotent stem/progenitor cells (e.g., CD34+ cells) so as to disrupt the expression of one or more genes are detailed in U.S. Pat. No. 8,518,701; US 2010/0251395; and US 2012/0222143, the disclosures of each of which are incorporated herein by reference in their entirety (in case of conflict, the instant specification is controlling).

As used herein, the terms “effective amount,” “therapeutically effective amount,” and a “sufficient amount” of composition, vector construct, viral vector, or cell described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating Gaucher disease, it is an amount of the composition, RNAi compound, small molecule compound, vector construct, viral vector, protein, or cell sufficient to achieve a treatment response as compared to the response obtained without administration of the composition, RNAi compound, small molecule compound, vector construct, viral vector, protein, or cell. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, vector construct, viral vector, or cell of the present disclosure is an amount which results in a beneficial or desired result in a subject as compared to a control. As defined herein, a therapeutically effective amount of a composition, vector construct, viral vector, or cell of the present disclosure may be readily determined by one of ordinary skill by routine methods known in the art. Dosage regime may be adjusted to provide the optimum therapeutic response.

As used herein, the terms “embryonic stem cell” and “ES cell” refer to an embryo-derived totipotent or pluripotent stem cell, derived from the inner cell mass of a blastocyst that can be maintained in an in vitro culture under suitable conditions. ES cells are capable of differentiating into cells of any of the three vertebrate germ layers, e.g., the endoderm, the ectoderm, or the mesoderm. ES cells are also characterized by their ability propagate indefinitely under suitable in vitro culture conditions. See, for example, Thomson et al., Science 282:1145 (1998).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. Expression of a gene of interest in a subject can manifest, for example, by detecting: an increase in the quantity or concentration of mRNA encoding a corresponding protein (as assessed, e.g., using RNA detection procedures described herein or known in the art, such as quantitative polymerase chain reaction (qPCR) and RNA seq techniques), an increase in the quantity or concentration of a corresponding protein (as assessed, e.g., using protein detection methods described herein or known in the art, such as enzyme-linked immunosorbent assays (ELISA), among others), and/or an increase in the activity of a corresponding protein (e.g., in the case of an enzyme, as assessed using an enzymatic activity assay described herein or known in the art) in a sample obtained from the subject.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “2A” refers to the 2A nucleic acid sequence of the food-and-mouth disease virus (F2A), equine rhinitis A virus (E2A), porcine teschovirus (P2A), or Thosea asigna virus (T2A). In general, an 2A sequence is a feature that allows the coordinated expression of multiple proteins in equimolar amounts from a single open reading frame. 2A mediates a cotranslational cleavage event, which separates proteins linked by 2A sequences. Multiple 2A sequences may be used in one vector, and co-expression of proteins linked by 2A will work in most eukaryotic cells as cleavage activity depends on eukaryotic ribosomes. For an example of the use of 2A to express multiple proteins, see Ryan and Drew, EMBO Journal 13:928 (1994), the disclosure of which is incorporated herein by reference. Exemplary 2A sequences include F2A, P2A, E2A, and T2A sequences.

As used herein, the term “functional potential” as it pertains to a stem cell, such as a hematopoietic stem cell, refers to the functional properties of stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of stem cells to give rise to daughter cells that have equivalent potential as the mother cell, and further that this ability can repeatedly occur throughout the lifetime of an individual without exhaustion), and 3) the ability of stem cells or progeny thereof to be reintroduced into a transplant recipient whereupon they home to the stem cell niche and re-establish productive and sustained cell growth and differentiation.

As used herein, the term “furin-resistant IGF-II mutein” refers to an insulin-like growth factor II (IGF-II)-based peptide containing an altered amino acid sequence relative to wild-type IGF-II (SEQ ID NO: 22) that abolishes at least one native furin protease cleavage site or changes a sequence close or adjacent to a native furin protease cleavage site such that the furin cleavage is prevented, inhibited, reduced, or slowed down as compared to a wild-type human IGF-II peptide. As used herein, a furin-resistant IGF-II mutein is also referred to as an IGF-II mutein that is resistant to furin. Exemplary furin-resistant IGF-II muteins contain amino acid substitutions at positions corresponding to Arg37 of SEQ ID NO: 22.

As used herein, the term “furin protease cleavage site” (also referred to as “furin cleavage site” or “furin cleavage sequence”) refers to the amino acid sequence of a peptide or protein that serves as a recognition sequence for enzymatic protease cleavage by furin or furin-like proteases. Typically, a furin protease cleavage site has a consensus sequence Arg-X-X-Arg, where X is any amino acid. The cleavage site is positioned after the carboxy-terminal arginine (Arg) residue in the sequence. In some embodiments, a furin cleavage site has a consensus sequence Lys/Arg-X-X-X-Lys/Arg-Arg, where X is any amino acid. The cleavage site is positioned after the carboxy-terminal arginine (Arg) residue in the sequence.

As used herein, the term “furin” refers to any protease that can recognize and cleave the furin protease cleavage site as defined herein, including furin or furin-like protease. Furin is also known as paired basic amino acid cleaving enzyme (PACE). Furin belongs to the subtilisin-like proprotein convertase family. The gene encoding furin is known as FUR (FES Upstream Region).

As used herein, the term “fusion protein” refers to a protein that is joined via a covalent bond to another molecule. A fusion protein can be chemically synthesized by, e.g., an amide-bond forming reaction between the N-terminus of one protein to the C-terminus of another protein. Alternatively, a fusion protein containing one protein covalently bound to another protein can be expressed recombinantly in a cell (e.g., a mammalian cell) by expression of a polynucleotide encoding the fusion protein, for example, from a vector or the genome of the cell. A fusion protein may contain one protein that is covalently bound to a linker, which in turn is covalently bound to another molecule. Examples of linkers that can be used for the formation of a fusion protein include peptide-containing linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Linkers can be prepared using a variety of strategies that are well known in the field, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche, et al., Bioorg. Med. Chem. 20:571-582 (2012)).

As used herein, the term “Gaucher disease” refers to an autosomal recessive lysosomal storage disorder (LSD) that results from a deficiency in GBA, a lysosomal enzyme that mediates the metabolism of glycosphingolipids in the membranes of white blood cells and red blood cells. Gaucher disease is the most common form of LSD. Reduced levels of GBA in Gaucher disease results in excessive accumulation of glucocerebrosides in lysosomes of phagocytic cells, resulting in splenomegaly, hepatomegaly, bone disease, thrombocytopenia, anemia, fatigue, neurological symptoms (e.g., impaired olfaction, cognition, seizures, hypertonia, intellectual disability, apnea, dementia, and ocular muscle apraxia), and low blood platelet count. Gaucher disease is known to present clinically in one of three forms. Gaucher disease Type I refers to a non-neuropathic form of the disease, which is most common and least severe of the Gaucher variants. Type I patients typically present with symptoms in early life or adulthood, with symptoms generally being limited to the liver, spleen, and bone. Gaucher disease Type II refers to the acute infantile neuropathic variant of the disease. Type II patients experience symptoms within 6 months after birth, which include hepatomegaly, neurodegeneration, eye movement disorders, spasticity and seizures, limb rigidity, and a poor ability to suck and swallow. Type II patients generally do not live past the age of two. Gaucher disease Type Ill refers to the chronic neuropathic variant of the disease. Type III patients generally present with symptoms at various times in childhood or even in adulthood, which may include insidious neurological symptoms, hepatomegaly, seizures, poor motor coordination, bone disease, eye movement disorders, anemia, and respiratory problems. Type III patients generally live into their early teen years and adulthood.

As used herein, the term “glycosylation independent lysosomal targeting” or “GILT” refers to lysosomal targeting that is mannose-6-phosphate (M6P)-independent. A GILT tag may be used to target a protein (e.g., GBA) expressed as a GILT-tagged fusion protein (e.g., a GBA fusion protein coupled to an IGF-II mutein), to the lysosome. As used interchangeably herein, the terms “cation-independent mannose-6-phosphate receptor (CI-MPR),” “M6P/IGF-II receptor,” “CI-MPR/IGF-II receptor,” “IGF-II receptor” or “IGF2 Receptor,” or abbreviations thereof, refer to the cellular receptor which binds both M6P and IGF-II.

As used herein, patients suffering from “GBA-associated Gaucher disease” are those patients that have been diagnosed as having Gaucher disease and also contain a deleterious mutation in the GBA gene. GBA mutations are discussed in in Barkhuizen et al., Neurochemistry International 93:6 (2016), Riboldi et al. Cells 8:364 (2019), Hruska et al. Hum. Mutat. 29:567-83, (2008), and Sidransky et al., Lancet Neurol. 11:986 (2012), the disclosures of which are incorporated herein by reference as they pertain to human GBA mutations.

As used herein, the terms “glucocerebrosidase” and “GBA” refer to the lysosomal enzyme responsible for the metabolism of glucocerebroside (also known as glucosylceramide) to glucose and ceramide. The gene is located on chromosome 1q21 and is also known as GBA1. The terms “glucocerebrosidase” and “GBA” also refer to variants of wild-type glucocerebrosidase enzymes and nucleic acids encoding the same, such as variant proteins having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the amino acid sequence of a wild-type GBA enzyme (e.g., SEQ ID NO: 1) or polynucleotides having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the nucleic acid sequence of a wild-type GBA gene (e.g., SEQ ID NO: 6), provided that the GBA analog encoded retains the therapeutic function of wild-type GBA. “GBA” may also refer to a GBA protein in which the natural signal peptide is present. The terms “glucocerebrosidase” and “GBA” may also refer to codon-optimized polynucleotides encoding GBA, such as codon-optimized polynucleotides having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the nucleic acid sequence of SEQ ID NO: 3. Alternatively, “GBA” may refer to a GBA protein in which the natural signal peptide has been removed (e.g., the mature protein). GBA may also refer to the catalytic domain of GBA, such as a domain containing residues 76-381 and 416-430 of SEQ ID NO: 1, or a variant having at least 85% sequence identity (e.g., 85%, 86%, 87%, 88%, 89%, 90%, 91%, 92%, 93%, 94%, 95%, 96%, 97%, 98%, 99%, or greater, sequence identity) to such a domain. As used herein, GBA may refer to the lysosomal enzyme or the gene encoding this protein, depending upon the context, as will be appreciated by one of skill in the art.

As used herein, the terms “hematopoietic stem cells” and “HSCs” refer to immature blood cells having the capacity to self-renew and to differentiate into mature blood cells of diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSC (LT-HSC) and short-term repopulating HSC (ST-HSC). LT-HSC and ST-HSC are differentiated, based on functional potential and on cell surface marker expression. For example, human HSC are a CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSC are CD34−, SCA-1+, C-kit+, CD135−, Slamf1/CD150+, CD48−, and lin− (negative for mature lineage markers including Ter119, CD11b, Gr1, CD3, CD4, CD8, B220, IL-7ra), whereas ST-HS Care CD34+, SCA-1+, C-kit+, CD135−, Slamf1/CD150+, and lin− (negative for mature lineage markers including Ter 19, CD11 b, Gr1, CD3, CD4, CD8, B220, IL-7ra). In addition, ST-HSC are less quiescent (i.e., more active) and more proliferative than LT-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSCs can be used in any of the methods described herein. Optionally, ST-HSCs are useful because they are highly proliferative and thus, can more quickly give rise to differentiated progeny.

As used herein, the term “HLA-matched” refers to a donor-recipient pair in which none of the HLA antigens are mismatched between the donor and recipient, such as a donor providing a hematopoietic stem cell graft to a recipient in need of hematopoietic stem cell transplant therapy. HLA-matched (i.e., where all of the 6 alleles are matched) donor-recipient pairs have a decreased risk of graft rejection, as endogenous T cells and NK cells are less likely to recognize the incoming graft as foreign, and are thus less likely to mount an immune response against the transplant.

As used herein, the term “HLA-mismatched” refers to a donor-recipient pair in which at least one HLA antigen, in particular with respect to HLA-A, HLA-B, HLA-C, and HLA-DR, is mismatched between the donor and recipient, such as a donor providing a hematopoietic stem cell graft to a recipient in need of hematopoietic stem cell transplant therapy. In some embodiments, one haplotype is matched and the other is mismatched. HLA-mismatched donor-recipient pairs may have an increased risk of graft rejection relative to HLA-matched donor-recipient pairs, as endogenous T cells and NK cells are more likely to recognize the incoming graft as foreign in the case of an HLA-mismatched donor-recipient pair, and such T cells and NK cells are thus more likely to mount an immune response against the transplant.

As used herein, the terms “induced pluripotent stem cell,” “iPS cell,” and “iPSC” refer to a pluripotent stem cell that can be derived directly from a differentiated somatic cell. Human iPS cells can be generated by introducing specific sets of reprogramming factors into a non-pluripotent cell that can include, for example, Oct3/4, Sox family transcription factors (e.g., Sox1, Sox2, Sox3, Sox15), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. See, for example, Takahashi and Yamanaka, Cell 126:663 (2006).

As used herein, the terms “induced pluripotent stem cell,” “iPS cell,” and “iPSC” refer to a pluripotent stem cell that can be derived directly from a differentiated somatic cell. Human iPS cells can be generated by introducing specific sets of reprogramming factors into a non-pluripotent cell that can include, for example, Oct3/4, Sox family transcription factors (e.g., Sox1, Sox2, Sox3, Sox15), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. See, for example, Takahashi and Yamanaka, Cell 126:663 (2006).

As used herein, the term “IRES” refers to an internal ribosomal entry site. In general, an IRES sequence is a feature that allows eukaryotic ribosomes to bind an mRNA transcript and begin translation without binding to a 5′ capped end. An mRNA containing an IRES sequence produces two translation products, one initiating form the 5′ end of the mRNA and the other from an internal translation mechanism mediated by the IRES.

As used herein, the term “lymphoid cell” refers to white blood cells of the lymphoid lineage that are derived from a common lymphoid progenitor cell. Lymphoid cells are generally part of the adaptive immunity arm of the immune system and include cells such as NK cells, B-cells, and T-cells. As used herein, lymphoid cells refers to a population of cells that cannot differentiate into cells of the myeloid lineage (e.g., MPCs, erythrocytes, mast cells, megakaryocytes, thrombocytes, myeloblasts, basophils, neutrophils, eosinophils, monocytes, and macrophages).

As used herein, the term “macrophage” refers to a type of white blood cell that engulfs and digests cellular debris, foreign substances, microbes, cancer cells, and anything else that does not have 15 the types of proteins specific to healthy body cells on its surface in a process called phagocytosis. Macrophages are found in essentially all tissues, where they patrol for potential pathogens by amoeboid movement. They take various forms (with various names) throughout the body (e.g., histiocytes, Kupffer cells, alveolar macrophages, microglia, and others), but all are part of the mononuclear phagocyte system. Besides phagocytosis, they play a critical role in non-specific defense (innate immunity) and also 20 help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines.

As used herein, the terms “microglia” or “microglial cell” refer to a type of neuroglial cell found in the brain and spinal cord that function as resident macrophage cells and the principal line of immune defense in the central nervous system. Primary functions of microglial cells include immune surveillance, phagocytosis, extracellular signaling (e.g., production and release of cytokines, chemokines, prostaglandins, and reactive oxygen species), antigen presentation, and promotion of tissue repair and regeneration.

As used herein, the term “microglial progenitor cell” refers to a precursor cell that gives rise to microglial cells. Microglial precursor cells originate in the yolk sac during a limited period of embryonic development, infiltrate the brain mesenchyme, and perpetually renew themselves throughout life.

As used herein, the term “miRNA targeting sequence” refers to a nucleotide sequence located in the 3′-UTR of a target mRNA molecule which is complementary to a specific miRNA molecule (e.g. miR-126) such that they may hybridize and promote RNA-induced silencing complex-dependent and Dicer-dependent mRNA destabilization and/or cleavage, thereby preventing the expression of an mRNA transcript.

As used herein, the term “MND promoter” refers to a synthetic, constitutively active promoter derived from a myeloproliferative sarcoma virus (MSV). An MND promoter contains an MSV enhancer, a U3 region of a Maloney Murine Leukemia Virus, a deletion of a negative control region, and a substitution of a dl587rev primer binding site. An MND promoter may have, for example, the nucleic acid sequence of SEQ ID NO: 20 or 21 or may be a variant thereof having at least 85% (e.g., at least 85%, 90%, 95%, 96%, 97%, 98%, 99%, or more) sequence identity to the nucleic acid sequence of SEQ ID NO: 20 or 21.

According to the disclosed methods and compositions, the MND promoter is suitable for incorporation into a transgene expression construct (e.g., a plasmid or viral vector) for driving expression of one or more transgenes in one or more target cell types.

As used herein, the term “monocistronic” refers to an RNA or DNA construct that contains the coding sequence for a single protein or polypeptide product.

As used herein, the term “myeloablative” or “myeloablation” refers to a conditioning regiment that substantially impairs or destroys the hematopoietic system, typically by exposure to a cytotoxic agent (e.g., busulfan) or radiation. Myeloablation encompasses complete myeloablation brought on by high doses of cytotoxic agent or total body irradiation that destroys the hematopoietic system.

As used herein, the term “non-myeloablative” or “myelosuppressive” refers to a conditioning regiment that does not eliminate substantially all hematopoietic cells of host origin.

As used herein, the term “monocyte” refers to a type of white blood cell (i.e., a leukocyte) that is capable of differentiating into macrophages and myeloid lineage dendritic cells. Monocytes constitute an important component of the vertebrate adaptive immune response. Three different types of monocytes are known to exist, including classical monocytes characterized by strong expression of the CD14 cell surface receptor and no CD16 expression (i.e., CD14++CD16−), non-classical monocytes exhibiting low levels of CD14 expression and co-expression of C16 (CD14+CD16+), and intermediate monocytes exhibiting high levels of CD14 expression and low levels of C16 expression (CD14++CD16+). Monocytes perform a variety of functions that serve the immune system, including phagocytosis, antigen presentation, and cytokine secretion.

As used herein, the term “multipotent cell” refers to a cell that possesses the ability to develop into multiple (e.g., 2, 3, 4, 5, or more) but not all differentiated cell types. Non-limiting examples of multipotent cells include cells of the hematopoietic lineage (e.g., granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of multipotent cells are CD34+ cells.

As used herein, the term “mutation” refers to a change in the nucleotide sequence of a gene. Mutations in a gene may occur naturally as a result of, for example, errors in DNA replication, DNA repair, irradiation, and exposure to carcinogens or mutations may be induced as a result of administration of a transgene expressing a mutant gene. Mutations may result from a single nucleotide substitution or deletion. The nomenclature for describing mutations and sequence variations uses the format “reference sequence.code,” wherein the reference sequence may be “c,” designating a coding DNA and the code may contain symbols including “>,” designating a single nucleotide substitution, “del,” designating a deletion, “ins” designating an insertion, or may contain “a+b” in reference to substitutions occurring within an intron, wherein x denotes a number corresponding to a nucleotide within the coding DNA sequence (e.g., a nucleotide within an exon of a coding DNA sequence) and y corresponds to the number of nucleotides 3′ relative to x. For example, the GBA mutant associated with a substitution described as c.84insG mutation has a G inserted at nucleotide position 84 of the GBA coding DNA sequence. Mutations may also result in a substitution of a single amino acid within the peptide chain. The nomenclature for describing mutations resulting amino acid substitutions uses the format “p.AnB,” where “p” designates the variation at the level of the protein, “A” designates the amino acid found in the wild-type variant of the protein, “n” designates the number of the amino acid within the peptide chain, and “B” designates the new amino acid that resulted from the substitution. For example, a p.N370S variant of the GBA gene corresponds to a change in the protein at amino acid 370 where an asparagine is substituted for serine.

As used herein, the term “myeloid cells” refers to blood cells derived from the bone marrow that belong to the myeloid cell lineage and arise from common myeloid progenitor cells that gives rise to granulocytes, monocytes, erythrocytes, and platelets. Non-limiting examples of myeloid cells include MPCs, erythrocytes, mast cells, megakaryocytes, thrombocytes, myeloblasts, basophils, neutrophils, eosinophils, monocytes, and macrophages. As used herein, myeloid cells refers specifically to a group of myeloid lineage cells that are not capable if differentiating into cells of the lymphoid lineage (e.g., NK cells, T-cells, B-cells, or plasma cells).

As used herein, the term “polycistronic” refers to an RNA or DNA construct that contains the coding sequence for more than one protein or polypeptide product. Exemplary polycistronic vectors include those described in WO 1993/003143, Ryan and Drew, EMBO Journal 13:928 (1994), Szymczak and Vignali, Expert Opin Biol Ther. 5:627 (2005), Szymczak et al., Nat Biotechnol. 22:589 (2004), and Osborn et al., Molecular Therapy 12:569 (2005).

As used herein, the term “pluripotent cell” refers to a cell that possesses the ability to develop into more than one differentiated cell type, such as a cell type of the hematopoietic lineage (e.g., granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of pluripotent cells are embryonic stem cells (ES cells) and induced pluripotent stem cells (iPS cells or iPSCs). For example, exemplary pluripotent cells include CD34+ cells.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, a potent “receptor-binding peptide (Rb) derived from ApoE”, has the ability to translocate proteins across the BBB into the brain when engineered as fusion proteins. This method can therefore function to selectively open the BBB for therapeutic agents (e.g., soluble GBA and/or SCARB2) when engineered as a fusion protein. This peptide can be readily attached to diagnostic or therapeutic agents without jeopardizing their biological functions or interfering with the important biological functions of ApoE due to the utilization of the Rb domain of ApoE, rather than the entire ApoE protein. This pathway is also an alternative uptake pathway that can facilitate further/secondary distribution within the brain after the agents reach the CNS due to the widespread expression of LDLRf members in brain parenchyma. Exemplary Rb domains can be found in the N-terminus of ApoE. For example, Rb domains useful in conjunction with the compositions and methods described herein are polypeptides having the amino acid sequence of residues 1 to 191 of SEQ ID NO: 29, residues 25 to 185 of SEQ ID NO: 29, residues 50 to 180 of SEQ ID NO: 29, residues 75 to 175 of SEQ ID NO: 29, residues 100 to 170 of SEQ ID NO: 29, or residues 125 to 165 of SEQ ID NO: 29, as well as variants thereof, such as polypeptides having at least 85% sequence identity (e.g., 85%, 86%, 87%, 88%, 89%, 90%, 91%, 92%, 93%, 94%, 95%, 96%, 97%, 98%, 99%, or greater, sequence identity) with respect to any of these sequences. An exemplary Rb domain is the region of ApoE having the amino acid sequence of residues 159 to 167 of SEQ ID NO: 29.

As used herein, the term “regulatory sequence” includes promoters, enhancers and other expression control elements (e.g., polyadenylation signals) that control the transcription or translation of the antibody chain genes. Such regulatory sequences are described, for example, in Goeddel, Gene Expression Technology: Methods in Enzymology 185 (Academic Press, San Diego, CA, (1990)); incorporated herein by reference.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the term “secretory signal peptide” refers to a short (usually between 16-60 amino acids) peptide region within the precursor protein that directs secretion of the precursor protein from the cytoplasm of the host into the periplasmic space or into the extracellular space. Such secretory signal peptides are generally located at the amino terminus of the precursor protein. In some embodiments, the secretory signal peptide is linked to the amino terminus and may be heterologous to the protein to which it is linked. Typically, secretory signal peptides are cleaved during transit through the cellular secretion pathway. Cleavage is not essential as long as the secreted protein retains its desired activity. Exemplary secretory signal peptides include those from IGF-II, alpha-1 antitrypsin, and GBA.

As used herein, the term “signal peptide” refers to a short (usually between 16-60 amino acids) polypeptide present on precursor proteins (typically at the N terminus), which is typically absent from the mature protein. The signal peptide directs the transport of the translated protein through the cell membrane. Signal peptides may also be called targeting signals, transit peptides, localization signals, or signal sequences. For example, the signal sequence may be a co-translational or post-translational signal peptide. Exemplary signal peptides include the GBA signal peptide (e.g., a 39-amino acid GBA signal peptide as described in Sorge et al., Am. J. Hum. Genet. 41:1016-1024 (1987); incorporated herein by reference).

As used herein, the terms “stem cell” and “undifferentiated cell” refer to a cell in an undifferentiated or partially differentiated state that has the developmental potential to differentiate into multiple cell types. A stem cell is capable of proliferation and giving rise to more such stem cells while maintaining its functional potential. Stem cells can divide asymmetrically, which is known as obligatory asymmetrical differentiation, with one daughter cell retaining the functional potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. Alternatively, some of the stem cells in a population can divide symmetrically into two stem cells. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection, and the like.

As used herein, the term “transgene” refers to a recombinant nucleic acid (e.g., DNA or cDNA) encoding a gene product (e.g., GBA and/or SCARB2). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s) and/or other functional elements. Embodiments of the invention may utilize any known suitable promoter, enhancer(s), destabilizing domain(s), response element(s), reporter element(s), insulator element(s), polyadenylation signal(s), and/or other functional elements.

As used herein, the terms “subject” and “patient” refer to an animal (e.g., a mammal, such as a human). A subject to be treated according to the methods described herein may be one who has been diagnosed with Gaucher disease or GBA-associated Gaucher disease, or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transduction” and “transduce” refer to a method of introducing a vector construct or a part thereof into a cell. Wherein the vector construct is contained in a viral vector such as for example a lentiviral vector, transduction refers to viral infection of the cell, and subsequent transfer and integration of the vector construct or part thereof into the cell genome.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” includes a nucleic acid vector, e.g., a DNA vector, such as a plasmid, a RNA vector, virus, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/011026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of GBA and/or SCARB2, as described herein, include plasmids that contain regulatory sequences, such as promoter and enhancer regions, which direct gene transcription. Other useful vectors for expression of GBA and/or SCARB2 contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES) or 2A sequence (e.g., P2A, E2A, or T2A), and polyadenylation signal site to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin.

### DETAILED DESCRIPTION

Described herein are compositions and methods for the treatment of Gaucher disease in a subject (such as a mammalian subject, for example, a human). Using the compositions and methods described herein, one can treat Gaucher disease in a subject (e.g., a human subject) by administering one or more agents that collectively increase the expression of β-glucocerebrosidase (GBA) and/or scavenger receptor class B member 2 (SCARB2). Exemplary agents may include one or more polynucleotides containing a transgene that encodes a GBA protein and/or a SCARB2 protein, one or more interfering RNA (RNAi) molecules that collectively increase expression and/or activity of GBA and/or SCARB2, and one or more small molecules that collectively increase expression and/or activity of the GBA and/or SCARB2 protein. The present disclosure also provides pluripotent cells, such as CD34+ cells, that express a transgene containing one or more of the aforementioned polynucleotides. For example, described herein are compositions containing pluripotent cells that have been modified ex-vivo to express GBA and/or SCARB2. The sections that follow describe the compositions and methods useful for the treatment of Gaucher disease in further detail.

The present disclosure is based, in part, on the observation that concomitantly elevating expression levels of GBA and SCARB2 in target hematopoietic cells improves GBA enzymatic activity, likely as a result of improved trafficking of GBA to the lysosome by SCARB2. These findings suggest that that increasing the expression and/or activity levels GBA and SCARB2 together can be used to treat Gaucher disease, which is characterized by GBA deficiency. In view of this surprising observation, a single therapeutic product, such as a single population of cells, viral vectors, or other agents, promoting the expression and/or activity of GBA and SCARB2 may be used to treat Gaucher patients with enhanced efficacy relative to a product promoting the expression and/or activity of either agent alone. Using traditional monotherapy methods, each patient would require a customized agent that delivers only the gene or protein for which the patient is deficient. The present compositions and methods provide the unexpected technical advantage of being able to treat Gaucher disease using a single product that augments the expression and/or activity of GBA and SCARB2, even if the patient is deficient in only one of the proteins.

Exemplary agents that may be used to elevate GBA and SCARB2 expression and/or activity levels in accordance with the methods of the disclosure include, without limitation, populations of cells (e.g., cells, such as CD34+ cells, hematopoietic stem cells, or myeloid progenitor cells) that contain nucleic acids encoding the GBA and/or SCARB2 proteins (e.g., nucleic acids capable of expression in macrophages or microglia), viral vectors that encode one or more of the desired proteins, and nucleic acid molecules, such as interfering RNA molecules, that stimulate the endogenous expression of GBA and/or SCARB2 proteins. Additional examples of agents that may be used for this purpose include pharmaceutical compositions containing the one or more proteins themselves.

### Gaucher Disease

Gaucher disease refers to an autosomal recessive lysosomal storage disorder (LSD) that results from a deficiency in GBA, a lysosomal enzyme that mediates the metabolism of glycosphingolipids in the membranes of white blood cells and red blood cells. Gaucher disease is the most common form of LSD. Reduced levels of GBA in Gaucher disease results in excessive accumulation of glucocerebrosides in lysosomes of phagocytic cells, resulting in splenomegaly, hepatomegaly, bone disease, thrombocytopenia, anemia, fatigue, neurological symptoms (e.g., impaired olfaction, cognition, seizures, hypertonia, intellectual disability, apnea, dementia, and ocular muscle apraxia), and low blood platelet count.

Gaucher disease is known to present clinically in one of three forms. Gaucher disease Type I refers to a non-neuropathic form of the disease, which is most common and least severe of the Gaucher variants. Type I patients typically present with symptoms in early life or adulthood, with symptoms generally being limited to the liver, spleen, and bone. A key feature of Type I disease is the presence of Gaucher cells, macrophages containing excessively large lysosomes. Gaucher disease Type II refers to the acute infantile neuropathic variant of the disease. Type II patients experience symptoms within 6 months after birth, which include hepatomegaly, neurodegeneration, eye movement disorders, spasticity and seizures, limb rigidity, and a poor ability to suck and swallow. Type II patients generally do not live past the age of two. Gaucher disease Type Ill refers to the chronic neuropathic variant of the disease. Type III patients generally present with symptoms at various times in childhood or even in adulthood, which may include insidious neurological symptoms, hepatomegaly, seizures, poor motor coordination, bone disease, eye movement disorders, anemia, and respiratory problems. Type III patients generally live into their early teen years and adulthood.

### GBA-Associated Gaucher Disease

Recent studies have shown a link between mutations in the GBA gene and increased risk of Gaucher disease, with more severe mutations imparting higher levels of risk. Over 300 mutations in the GBA gene have been identified in connection with Gaucher disease (Riboldi et al. Cells 8:364 (2019)), including 203 missense mutations, 18 nonsense mutations, 36 small insertions or deletions that result in frameshifts or in-frame alterations, 14 splice junction mutations, and 13 complex alleles having two or more mutations in cis (Hruska et al. Hum. Mutat. 29:567-83, (2008)). Non-limiting examples of GBA mutations associated with Gaucher disease include the p.N370S substitution, p.R463C substitution, p.L444P substitution, p.D409H substitution, p.R463C substitution, p.R496H substitution, p. F252I substitution, p.A456P substitution, p.V460V substitution, p.V394L, p.E326K substitution, p.G377S substitution, p.N188S substitution, c.84insG insertion, c.84dupG (84GG) duplication, c.115+1G>A substitution, or c.IVS2DS+1 G-A splice site mutation. GBA mutations are discussed in in Barkhuizen et al., Neurochemistry International 93:6 (2016), Sidransky and Lopez, Lancet Neurol. 11:986 (2012), and Riboldi et al. Cells 8:364 (2019), the disclosures of which are incorporated herein by reference as they pertain to human GBA mutations. These mutations may also elicit a gain of toxic function by activating endoplasmic reticulum (ER) stress as the mutant protein is trapped in the ER. Markers of ER stress are elevated in Gaucher disease brains with GBA mutations, and dysregulation of ER calcium stores have been reported in cell models containing GBA mutations associated with Gaucher disease. Additionally, these mutants could increase the total burden of to-be-degraded misfolded polypeptides in neural cells resulting in altered cellular function due to a diversion of cellular resources. GBA mutations resulting in a gain of toxic function and/or altered cellular function due to a diversion of cellular resources are discussed in Gregg et al., Ann. Neurol. 72:455-463 (2012), Schöndorf et al., Nat. Commun. 5:4028 (2014), Kilpatrick et al., Cell Calcium 59:12-20 (2016), and Cullen et al., Ann. Neurol. 69:940-953 (2011), the disclosure of which are incorporated herein by reference as they pertain to human GBA mutations.

Treatments for Gaucher disease have long focused on ameliorating the symptomology of the condition, without addressing the underlying biological cause of the disease. Unlike these treatments, the methods described herein provide the benefit of treating a different biochemical phenomenon that can underlie the development of Gaucher disease. As such, the compositions and methods described herein target the physiological cause of the disease, representing a potential curative therapy. The compositions and methods described herein can be used to treat Gaucher disease by administering one or more agents (e.g., polynucleotides encoding GBA and/or SCARB2 or pluripotent cells (e.g., CD34+ cells) that express the same, RNAi agents or small molecule compounds that increase GBA and/or SCARB2 expression and/or activity, and/or recombinant GBA and/or SCARB2 proteins. These compositions and methods can be used to treat Gaucher disease with any etiology, e.g., genetic mutation, environmental toxin, or sporadic. These compositions and methods can also be used to treat patients with GBA-associated Gaucher disease, e.g., Gaucher disease associated with a mutation in the GBA gene. The compositions and methods described herein can be used to treat patients with normal GBA activity, reduced GBA activity, and patients whose GBA mutational status and/or GBA activity level is unknown. The compositions and methods described herein may also be administered as a preventative treatment to patients at risk of developing Gaucher disease, e.g., patients with a GBA mutation, or patients with reduced GBA activity. The cells administered to patients suffering from Gaucher disease can express SCARB2 in addition to GBA.

### GBA Sequences

GBA-encoding constructs that may be used in conjunction with the compositions and methods described herein include transgenes comprising polynucleotides that encode wild-type GBA (the amino acid sequence of which is shown as SEQ ID NO: 1, below) or a variant thereof, such as a polynucleotide that encodes a protein having at least 85% sequence identity (e.g., 85%, 90%, 95%, 96%, 97%, 98%, 99%, or more, sequence identity) to the amino acid sequence of SEQ ID NO: 1. In some embodiments, the GBA-encoding constructs include polynucleotides that encode the catalytic domain of GBA, such as a domain containing residues 76-381 and 416-430 of SEQ ID NO: 1. In some embodiments, the GBA-encoding constructs include polynucleotides having at least 85% sequence identity (e.g., 85%, 90%, 95%, 96%, 97%, 98%, 99%, or more, sequence identity) to the nucleic acid sequence of SEQ ID NO: 6.

In some embodiments, the GBA-encoding constructs may be codon-optimized polynucleotides having at least 85% sequence identity (e.g., 85%, 90%, 95%, 96%, 97%, 98%, 99%, or more, sequence identity) to the nucleic acid sequence of SEQ ID NO: 7, so to confer resistance against degradation by nucleases and inhibitory RNAs directed to endogenous GBA.

In some embodiments, the transgene encoding GBA encodes a GBA fusion protein. In some embodiments, the GBA fusion protein has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of SEQ ID NO: 2. In some embodiments, the GBA fusion protein has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of SEQ ID NO: 3. In some embodiments, the GBA fusion protein has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of SEQ ID NO: 4.

In some embodiments, the transgene encoding the GBA fusion protein has a nucleic acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO: 8. In some embodiments, the transgene encoding the GBA fusion protein has a nucleic acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO: 9. In some embodiments, the transgene encoding the GBA fusion protein has a nucleic acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO: 10. In some embodiments, the transgene encoding the GBA fusion protein has a nucleic acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO: 11.

Wild-type human GBA (GenBank accession number: AAC63056.1) has the amino acid sequence of:

(SEQ ID NO: 1)
MEFSSPSREECPKPLSRVSIMAGSLTGLLLLQAVSWASGARPCIPKSFGYSSVVCVCNATYCDSFDPPTF
PALGTFSRYESTRSGRRMELSMGPIQANHTGTGLLLTLQPEQKFQKVKGFGGAMTDAAALNILALSPPAQ
NLLLKSYFSEEGIGYNIIRVPMASCDFSIRTYTYADTPDDFQLHNFSLPEEDTKLKIPLIHRALQLAQRP
VSLLASPWTSPTWLKTNGAVNGKGSLKGQPGDIYHQTWARYFVKFLDAYAEHKLQFWAVTAENEPSAGLL
SGYPFQCLGFTPEHQRDFIARDLGPTLANSTHHNVRLLMLDDQRLLLPHWAKVVLTDPEAAKYVHGIAVH
WYLDFLAPAKATLGETHRLFPNTMLFASEACVGSKFWEQSVRLGSWDRGMQYSHSIITNLLYHVVGWTDW
NLALNPEGGPNWVRNFVDSPIIVDITKDTFYKQPMFYHLGHFSKFIPEGSQRVGLVASQKNDLDAVALMH
PDGSAVVVVLNRSSKDVPLTIKDPAVGFLETISPGYSIHTYLWRRQ
The GBA fusion protein has the amino acid sequence of:
(SEQ ID NO: 2)
MEFSSPSREECPKPLSRVSIMAGSLTGLLLLQAVSWASGARPCIPKSFGYSSVVCVCNATYCDSFDPPTF
PALGTFSRYESTRSGRRMELSMGPIQANHTGTGLLLTLQPEQKFQKVKGFGGAMTDAAALNILALSPPAQ
NLLLKSYFSEEGIGYNIIRVPMASCDFSIRTYTYADTPDDFQLHNFSLPEEDTKLKIPLIHRALQLAQRP
VSLLASPWTSPTWLKTNGAVNGKGSLKGQPGDIYHQTWARYFVKFLDAYAEHKLQFWAVTAENEPSAGLL
SGYPFQCLGFTPEHQRDFIARDLGPTLANSTHHNVRLLMLDDQRLLLPHWAKVVLTDPEAAKYVHGIAVH
WYLDFLAPAKATLGETHRLFPNTMLFASEACVGSKFWEQSVRLGSWDRGMQYSHSIITNLLYHVVGWTDW
NLALNPEGGPNWVRNFVDSPIIVDITKDTFYKQPMFYHLGHFSKFIPEGSQRVGLVASQKNDLDAVALMH
PDGSAVVVVLNRSSKDVPLTIKDPAVGFLETISPGYSIHTYLWRRQGGGGAGGGGAGGGGAGGGGAGGGP
SLCGGELVDTLQFVCGDRGFYFSRPASRVSARSRGIVEECCFRSCDLALLETYCATPAKSE
Alternatively, the GBA fusion protein has the amino acid
sequence of:
(SEQ ID NO: 3)
MEFSSPSREECPKPLSRVSIMAGSLTGLLLLQAVSWASGARPCIPKSFGYSSVVCVCNATYCDSFDPPTF
PALGTFSRYESTRSGRRMELSMGPIQANHTGTGLLLTLQPEQKFQKVKGFGGAMTDAAALNILALSPPAQ
NLLLKSYFSEEGIGYNIIRVPMASCDFSIRTYTYADTPDDFQLHNFSLPEEDTKLKIPLIHRALQLAQRP
VSLLASPWTSPTWLKTNGAVNGKGSLKGQPGDIYHQTWARYFVKFLDAYAEHKLQFWAVTAENEPSAGLL
SGYPFQCLGFTPEHQRDFIARDLGPTLANSTHHNVRLLMLDDQRLLLPHWAKVVLTDPEAAKYVHGIAVH
WYLDFLAPAKATLGETHRLFPNTMLFASEACVGSKFWEQSVRLGSWDRGMQYSHSIITNLLYHVVGWTDW
NLALNPEGGPNWVRNFVDSPIIVDITKDTFYKQPMFYHLGHFSKFIPEGSQRVGLVASQKNDLDAVALMH
PDGSAVVVVLNRSSKDVPLTIKDPAVGFLETISPGYSIHTYLWRRQGAPGGGSPAPAPTPAPAPTPAPAG
GGPSGAPLCGGELVDTLQFVCGDRGFYFSRPASRVSARSRGIVEECCFRSCDLALLETYCATPAKSE
Alternatively, the GBA fusion protein has the amino acid
sequence of:
(SEQ ID NO: 4)
MEFSSPSREECPKPLSRVSIMAGSLTGLLLLQAVSWASGARPCIPKSFGYSSVVCVCNATYCDSFDPPTF
PALGTFSRYESTRSGRRMELSMGPIQANHTGTGLLLTLQPEQKFQKVKGFGGAMTDAAALNILALSPPAQ
NLLLKSYFSEEGIGYNIIRVPMASCDFSIRTYTYADTPDDFQLHNFSLPEEDTKLKIPLIHRALQLAQRP
VSLLASPWTSPTWLKTNGAVNGKGSLKGQPGDIYHQTWARYFVKFLDAYAEHKLQFWAVTAENEPSAGLL
SGYPFQCLGFTPEHQRDFIARDLGPTLANSTHHNVRLLMLDDQRLLLPHWAKVVLTDPEAAKYVHGIAVH
WYLDFLAPAKATLGETHRLFPNTMLFASEACVGSKFWEQSVRLGSWDRGMQYSHSIITNLLYHVVGWTDW
NLALNPEGGPNWVRNFVDSPIIVDITKDTFYKQPMFYHLGHFSKFIPEGSQRVGLVASQKNDLDAVALMH
PDGSAVVVVLNRSSKDVPLTIKDPAVGFLETISPGYSIHTYLWRRQGAPGGSPAGSPTSTEEGTSESATP
ESGPGTSTEPSEGSAPGSPAGSPTSTGPSGAPLCGGELVDTLQFVCGDRGFYFSRPASRVSARSRGIVEE
CCFRSCDLALLETYCATPAKSE
GBA protein having a modified signal peptide sequence
has the amino acid sequence of:
(SEQ ID NO: 5)
MGIPMGKSMLVLLTFLAFASCCIAARPCIPKSFGYSSVVCVCNATYCDSFDPPTFPALGTFSRYESTRSG
RRMELSMGPIQANHTGTGLLLTLQPEQKFQKVKGFGGAMTDAAALNILALSPPAQNLLLKSYFSEEGIGY
NIIRVPMASCDFSIRTYTYADTPDDFQLHNFSLPEEDTKLKIPLIHRALQLAQRPVSLLASPWTSPTWLK
TNGAVNGKGSLKGQPGDIYHQTWARYFVKFLDAYAEHKLQFWAVTAENEPSAGLLSGYPFQCLGFTPEHQ
RDFIARDLGPTLANSTHHNVRLLMLDDQRLLLPHWAKVVLTDPEAAKYVHGIAVHWYLDFLAPAKATLGE
THRLFPNTMLFASEACVGSKFWEQSVRLGSWDRGMQYSHSIITNLLYHVVGWTDWNLALNPEGGPNWVRN
FVDSPIIVDITKDTFYKQPMFYHLGHFSKFIPEGSQRVGLVASQKNDLDAVALMHPDGSAVVVVLNRSSK
DVPLTIKDPAVGFLETISPGYSIHTYLWRRQ
Wild-type human GBA (GenBank accession number: M19285.1)
has the nucleic acid sequence of:
(SEQ ID NO: 6)
GCTAACCTAGTGCCTATAGCTAAGGCAGGTACCTGCATCCTTGTTTTTGTTTAGTGGATCCTCTATCCTT
CAGAGACTCTGGAACCCCTGTGGTCTTCTCTTCATCTAATGACCCTGAGGGGATGGAGTTTTCAAGTCCT
TCCAGAGAGGAATGTCCCAAGCCTTTGAGTAGGGTAAGCATCATGGCTGGCAGCCTCACAGGTTTGCTTC
TACTTCAGGCAGTGTCGTGGGCATCAGGTGCCCGCCCCTGCATCCCTAAAAGCTTCGGCTACAGCTCGGT
GGTGTGTGTCTGCAATGCCACATACTGTGACTCCTTTGACCCCCCGACCTTTCCTGCCCTTGGTACCTTC
AGCCGCTATGAGAGTACACGCAGTGGGCGACGGATGGAGCTGAGTATGGGGCCCATCCAGGCTAATCACA
CGGGCACAGGCCTGCTACTGACCCTGCAGCCAGAACAGAAGTTCCAGAAAGTGAAGGGATTTGGAGGGGC
CATGACAGATGCTGCTGCTCTCAACATCCTTGCCCTGTCACCCCCTGCCCAAAATTTGCTACTTAAATCG
TACTTCTCTGAAGAAGGAATCGGATATAACATCATCCGGGTACCCATGGCCAGCTGTGACTTCTCCATCC
GCACCTACACCTATGCAGACACCCCTGATGATTTCCAGTTGCACAACTTCAGCCTCCCAGAGGAAGATAC
CAAGCTCAAGATACCCCTGATTCACCGAGCCCTGCAGTTGGCCCAGCGTCCCGTTTCACTCCTTGCCAGC
CCCTGGACATCACCCACTTGGCTCAAGACCAATGGAGCGGTGAATGGGAAGGGGTCACTCAAGGGACAGC
CCGGAGACATCTACCACCAGACCTGGGCCAGATACTTTGTGAAGTTCCTGGATGCCTATGCTGAGCACAA
GTTACAGTTCTGGGCAGTGACAGCTGAAAATGAGCCTTCTGCTGGGCTGTTGAGTGGATACCCCTTCCAG
TGCCTGGGCTTCACCCCTGAACATCAGCGAGACTTCATTGCCCGTGACCTAGGTCCTACCCTCGCCAACA
GTACTCACCACAATGTCCGCCTACTCATGCTGGATGACCAACGCTTGCTGCTGCCCCACTGGGCAAAGGT
GGTACTGACAGACCCAGAAGCAGCTAAATATGTTCATGGCATTGCTGTACATTGGTACCTGGACTTTCTG
GCTCCAGCCAAAGCCACCCTAGGGGAGACACACCGCCTGTTCCCCAACACCATGCTCTTTGCCTCAGAGG
CCTGTGTGGGCTCCAAGTTCTGGGAGCAGAGTGTGCGGCTAGGCTCCTGGGATCGAGGGATGCAGTACAG
CCACAGCATCATCACGAACCTCCTGTACCATGTGGTCGGCTGGACCGACTGGAACCTTGCCCTGAACCCC
GAAGGAGGACCCAATTGGGTGCGTAACTTTGTCGACAGTCCCATCATTGTAGACATCACCAAGGACACGT
TTTACAAACAGCCCATGTTCTACCACCTTGGCCACTTCAGCAAGTTCATTCCTGAGGGCTCCCAGAGAGT
GGGGCTGGTTGCCAGTCAGAAGAACGACCTGGACGCAGTGGCACTGATGCATCCCGATGGCTCTGCTGTT
GTGGTCGTGCTAAACCGCTCCTCTAAGGATGTGCCTCTTACCATCAAGGATCCTGCTGTGGGCTTCCTGG
AGACAATCTCACCTGGCTACTCCATTCACACCTACCTGTGGCATCGCCAGTGATGGAGCAGATACTCAAG
GAGGCACTGGGCTCAGCCTGGGCATTAAAGGGACAGAGTCAGCTCACACGCTGTCTGTGACTAAAGAGGG
CACAGCAGGGCCAGTGTGAGCTTACAGCGACGTAAGCCCAGGGGCAATGGTTTGGGTGACTCACTTTCCC
CTCTAGGTGGTGCCCAGGGCTGGAGGCCCCTAGAAAAAGATCAGTAAGCCCCAGTGTCCCCCCAGCCCCC
ATGCTTATGTGAACATGCGCTGTGTGCTGCTTGCTTTGGAAACTNGCCTGGGTCCAGGCCTAGGGTGAGC
TCACTGTCCGTACAAACACAAGATCAGGGCTGAGGGTAAGGAAAAGAAGAGACTAGGAAAGCTGGGCCCA
AAACTGGAGACTGTTTGTCTTTCCTAGAGATGCAGAACTGGGCCCGTGGAGCAGCAGTGTCAGCATCAGG
GGGGAAGCCTTAAAGCAGCAGCGGGTGTGCCCAGGCACCCAGATGATTCCTATGGCACCAGCCAGGAAAA
ATGGCAGCTCTTAAAGGAGAAAATGTTTGAGCCCA
The codon-optimized GBA construct has the nucleic acid
sequence of:
(SEQ ID NO: 7)
ATGGAATTCAGCAGCCCCAGCCGGGAGGAATGCCCCAAGCCCCTGAGCAGAGTGTCCATCATGGCCGGAT
CTCTGACCGGACTGCTGCTGCTGCAAGCCGTGTCTTGGGCCAGCGGCGCCAGACCTTGCATCCCCAAGAG
CTTCGGCTACAGCAGCGTCGTGTGCGTGTGCAACGCCACCTACTGCGACAGCTTCGACCCCCCTACCTTT
CCCGCCCTGGGCACCTTCAGCAGATACGAGAGCACCCGCAGCGGCAGACGGATGGAACTGAGCATGGGCC
CCATCCAGGCCAATCACACCGGAACCGGCCTGCTGCTGACCCTGCAGCCCGAGCAGAAATTCCAGAAAGT
GAAGGGCTTCGGCGGAGCCATGACAGACGCCGCTGCCCTGAACATCCTGGCCCTGAGCCCCCCTGCTCAG
AATCTGCTGCTCAAGAGCTACTTCAGCGAGGAAGGCATCGGCTACAACATCATCCGGGTGCCCATGGCTA
GCTGCGACTTCAGCATCCGGACCTACACCTACGCCGACACCCCCGACGACTTCCAGCTGCACAACTTCAG
CCTGCCCGAAGAGGACACCAAGCTGAAGATCCCCCTGATCCACAGAGCCCTGCAGCTGGCCCAGAGGCCT
GTGTCTCTGCTGGCTAGCCCTTGGACCAGCCCCACCTGGCTGAAAACCAACGGAGCCGTGAACGGCAAGG
GCAGCCTGAAGGGCCAGCCCGGCGACATCTACCACCAGACCTGGGCCCGGTACTTCGTGAAGTTCCTGGA
CGCCTATGCCGAGCACAAGCTGCAGTTCTGGGCCGTGACCGCCGAGAATGAGCCTAGCGCTGGACTGCTG
TCCGGCTACCCCTTCCAATGCCTGGGCTTCACACCCGAGCACCAGCGGGACTTTATCGCCAGAGATCTGG
GCCCCACACTGGCCAACAGCACCCACCACAACGTGCGGCTGCTGATGCTGGACGACCAGAGACTGCTGCT
CCCCCACTGGGCCAAGGTGGTGCTGACAGACCCCGAGGCCGCCAAATACGTCCACGGGATTGCCGTGCAC
TGGTATCTGGACTTTCTGGCCCCTGCCAAGGCCACACTGGGCGAGACACACCGGCTGTTCCCCAACACCA
TGCTGTTCGCCAGCGAGGCCTGCGTGGGCAGCAAGTTCTGGGAGCAGAGCGTGCGGCTGGGCAGCTGGGA
CAGAGGCATGCAGTACAGCCACAGCATCATCACCAACCTGCTGTACCACGTCGTGGGCTGGACCGACTGG
AATCTGGCCCTGAACCCTGAGGGAGGACCCAACTGGGTCCGCAACTTCGTGGACAGCCCCATCATCGTGG
ACATCACCAAGGACACCTTCTACAAGCAGCCCATGTTCTACCACCTGGGCCACTTCAGCAAGTTCATCCC
CGAGGGCAGCCAGAGAGTGGGACTGGTCGCCAGCCAGAAGAACGATCTGGACGCCGTGGCCCTGATGCAC
CCTGATGGCAGCGCTGTGGTGGTGGTCCTGAATCGGTCCAGCAAGGACGTGCCCCTGACCATCAAGGACC
CCGCCGTGGGCTTCCTGGAAACCATCAGCCCCGGCTACTCCATCCACACCTACCTGTGGCGGAGACAATG
A
The codon-optimized GBA fusion protein construct has
the nucleic acid sequence of:
(SEQ ID NO: 8)
ATGGAATTCAGCAGCCCCAGCCGGGAGGAATGCCCCAAGCCCCTGAGCAGAGTGTCCATCATGGCCGGAT
CTCTGACCGGACTGCTGCTGCTGCAAGCCGTGTCTTGGGCCAGCGGCGCCAGACCTTGCATCCCCAAGAG
CTTCGGCTACAGCAGCGTCGTGTGCGTGTGCAACGCCACCTACTGCGACAGCTTCGACCCCCCTACCTTT
CCCGCCCTGGGCACCTTCAGCAGATACGAGAGCACCCGCAGCGGCAGACGGATGGAACTGAGCATGGGCC
CCATCCAGGCCAATCACACCGGAACCGGCCTGCTGCTGACCCTGCAGCCCGAGCAGAAATTCCAGAAAGT
GAAGGGCTTCGGCGGAGCCATGACAGACGCCGCTGCCCTGAACATCCTGGCCCTGAGCCCCCCTGCTCAG
AATCTGCTGCTCAAGAGCTACTTCAGCGAGGAAGGCATCGGCTACAACATCATCCGGGTGCCCATGGCTA
GCTGCGACTTCAGCATCCGGACCTACACCTACGCCGACACCCCCGACGACTTCCAGCTGCACAACTTCAG
CCTGCCCGAAGAGGACACCAAGCTGAAGATCCCCCTGATCCACAGAGCCCTGCAGCTGGCCCAGAGGCCT
GTGTCTCTGCTGGCTAGCCCTTGGACCAGCCCCACCTGGCTGAAAACCAACGGAGCCGTGAACGGCAAGG
GCAGCCTGAAGGGCCAGCCCGGCGACATCTACCACCAGACCTGGGCCCGGTACTTCGTGAAGTTCCTGGA
CGCCTATGCCGAGCACAAGCTGCAGTTCTGGGCCGTGACCGCCGAGAATGAGCCTAGCGCTGGACTGCTG
TCCGGCTACCCCTTCCAATGCCTGGGCTTCACACCCGAGCACCAGCGGGACTTTATCGCCAGAGATCTGG
GCCCCACACTGGCCAACAGCACCCACCACAACGTGCGGCTGCTGATGCTGGACGACCAGAGACTGCTGCT
CCCCCACTGGGCCAAGGTGGTGCTGACAGACCCCGAGGCCGCCAAATACGTCCACGGGATTGCCGTGCAC
TGGTATCTGGACTTTCTGGCCCCTGCCAAGGCCACACTGGGCGAGACACACCGGCTGTTCCCCAACACCA
TGCTGTTCGCCAGCGAGGCCTGCGTGGGCAGCAAGTTCTGGGAGCAGAGCGTGCGGCTGGGCAGCTGGGA
CAGAGGCATGCAGTACAGCCACAGCATCATCACCAACCTGCTGTACCACGTCGTGGGCTGGACCGACTGG
AATCTGGCCCTGAACCCTGAGGGAGGACCCAACTGGGTCCGCAACTTCGTGGACAGCCCCATCATCGTGG
ACATCACCAAGGACACCTTCTACAAGCAGCCCATGTTCTACCACCTGGGCCACTTCAGCAAGTTCATCCC
CGAGGGCAGCCAGAGAGTGGGACTGGTCGCCAGCCAGAAGAACGATCTGGACGCCGTGGCCCTGATGCAC
CCTGATGGCAGCGCTGTGGTGGTGGTCCTGAATCGGTCCAGCAAGGACGTGCCCCTGACCATCAAGGACC
CCGCCGTGGGCTTCCTGGAAACCATCAGCCCCGGCTACTCCATCCACACCTACCTGTGGCGGAGACAAGG
CGGAGGCGGAGCTGGTGGCGGCGGAGCAGGCGGTGGTGGTGCAGGCGGCGGAGGTGCTGGCGGAGGACCA
TCTCTTTGTGGCGGAGAACTGGTGGACACCCTGCAGTTCGTGTGTGGCGACAGAGGCTTCTACTTTAGCA
GACCCGCCAGCAGAGTGTCCGCCAGATCTAGAGGAATCGTGGAAGAGTGCTGCTTCAGAAGCTGCGACCT
GGCACTGCTGGAAACCTACTGTGCCACACCAGCCAAGAGCGAGTGATGA
Alternatively, the codon-optimized human GBA fusion
protein has the nucleic acid sequence of:
(SEQ ID NO: 9)
ATGGAATTCAGCAGCCCCAGCCGGGAGGAATGCCCCAAGCCCCTGAGCAGAGTGTCCATCATGGCCGGAT
CTCTGACCGGACTGCTGCTGCTGCAAGCCGTGTCTTGGGCCAGCGGCGCCAGACCTTGCATCCCCAAGAG
CTTCGGCTACAGCAGCGTCGTGTGCGTGTGCAACGCCACCTACTGCGACAGCTTCGACCCCCCTACCTTT
CCCGCCCTGGGCACCTTCAGCAGATACGAGAGCACCCGCAGCGGCAGACGGATGGAACTGAGCATGGGCC
CCATCCAGGCCAATCACACCGGAACCGGCCTGCTGCTGACCCTGCAGCCCGAGCAGAAATTCCAGAAAGT
GAAGGGCTTCGGCGGAGCCATGACAGACGCCGCTGCCCTGAACATCCTGGCCCTGAGCCCCCCTGCTCAG
AATCTGCTGCTCAAGAGCTACTTCAGCGAGGAAGGCATCGGCTACAACATCATCCGGGTGCCCATGGCTA
GCTGCGACTTCAGCATCCGGACCTACACCTACGCCGACACCCCCGACGACTTCCAGCTGCACAACTTCAG
CCTGCCCGAAGAGGACACCAAGCTGAAGATCCCCCTGATCCACAGAGCCCTGCAGCTGGCCCAGAGGCCT
GTGTCTCTGCTGGCTAGCCCTTGGACCAGCCCCACCTGGCTGAAAACCAACGGAGCCGTGAACGGCAAGG
GCAGCCTGAAGGGCCAGCCCGGCGACATCTACCACCAGACCTGGGCCCGGTACTTCGTGAAGTTCCTGGA
CGCCTATGCCGAGCACAAGCTGCAGTTCTGGGCCGTGACCGCCGAGAATGAGCCTAGCGCTGGACTGCTG
TCCGGCTACCCCTTCCAATGCCTGGGCTTCACACCCGAGCACCAGCGGGACTTTATCGCCAGAGATCTGG
GCCCCACACTGGCCAACAGCACCCACCACAACGTGCGGCTGCTGATGCTGGACGACCAGAGACTGCTGCT
CCCCCACTGGGCCAAGGTGGTGCTGACAGACCCCGAGGCCGCCAAATACGTCCACGGGATTGCCGTGCAC
TGGTATCTGGACTTTCTGGCCCCTGCCAAGGCCACACTGGGCGAGACACACCGGCTGTTCCCCAACACCA
TGCTGTTCGCCAGCGAGGCCTGCGTGGGCAGCAAGTTCTGGGAGCAGAGCGTGCGGCTGGGCAGCTGGGA
CAGAGGCATGCAGTACAGCCACAGCATCATCACCAACCTGCTGTACCACGTCGTGGGCTGGACCGACTGG
AATCTGGCCCTGAACCCTGAGGGAGGACCCAACTGGGTCCGCAACTTCGTGGACAGCCCCATCATCGTGG
ACATCACCAAGGACACCTTCTACAAGCAGCCCATGTTCTACCACCTGGGCCACTTCAGCAAGTTCATCCC
CGAGGGCAGCCAGAGAGTGGGACTGGTCGCCAGCCAGAAGAACGATCTGGACGCCGTGGCCCTGATGCAC
CCTGATGGCAGCGCTGTGGTGGTGGTCCTGAATCGGTCCAGCAAGGACGTGCCCCTGACCATCAAGGACC
CCGCCGTGGGCTTCCTGGAAACCATCAGCCCCGGCTACTCCATCCACACCTACCTGTGGCGGAGACAAGG
AGCACCAGGCGGAGGATCTCCAGCTCCTGCTCCTACACCAGCTCCAGCACCGACGCCTGCTCCAGCTGGC
GGAGGACCTTCTGGTGCACCTCTTTGTGGCGGAGAGCTGGTGGATACCCTGCAGTTCGTGTGTGGCGACC
GGGGCTTCTACTTTAGCAGACCTGCCAGCAGAGTGTCCGCCAGATCTAGAGGCATCGTGGAAGAGTGCTG
CTTCAGAAGCTGCGACCTGGCACTGCTGGAAACCTACTGTGCCACACCAGCCAAGAGCGAGTGATGA
Alternatively, the codon-optimized human GBA fusion
protein has the nucleic acid sequence of:
(SEQ ID NO: 10)
ATGGAATTCAGCAGCCCCAGCCGGGAGGAATGCCCCAAGCCCCTGAGCAGAGTGTCCATCATGGCCGGAT
CTCTGACCGGACTGCTGCTGCTGCAAGCCGTGTCTTGGGCCAGCGGCGCCAGACCTTGCATCCCCAAGAG
CTTCGGCTACAGCAGCGTCGTGTGCGTGTGCAACGCCACCTACTGCGACAGCTTCGACCCCCCTACCTTT
CCCGCCCTGGGCACCTTCAGCAGATACGAGAGCACCCGCAGCGGCAGACGGATGGAACTGAGCATGGGCC
CCATCCAGGCCAATCACACCGGAACCGGCCTGCTGCTGACCCTGCAGCCCGAGCAGAAATTCCAGAAAGT
GAAGGGCTTCGGCGGAGCCATGACAGACGCCGCTGCCCTGAACATCCTGGCCCTGAGCCCCCCTGCTCAG
AATCTGCTGCTCAAGAGCTACTTCAGCGAGGAAGGCATCGGCTACAACATCATCCGGGTGCCCATGGCTA
GCTGCGACTTCAGCATCCGGACCTACACCTACGCCGACACCCCCGACGACTTCCAGCTGCACAACTTCAG
CCTGCCCGAAGAGGACACCAAGCTGAAGATCCCCCTGATCCACAGAGCCCTGCAGCTGGCCCAGAGGCCT
GTGTCTCTGCTGGCTAGCCCTTGGACCAGCCCCACCTGGCTGAAAACCAACGGAGCCGTGAACGGCAAGG
GCAGCCTGAAGGGCCAGCCCGGCGACATCTACCACCAGACCTGGGCCCGGTACTTCGTGAAGTTCCTGGA
CGCCTATGCCGAGCACAAGCTGCAGTTCTGGGCCGTGACCGCCGAGAATGAGCCTAGCGCTGGACTGCTG
TCCGGCTACCCCTTCCAATGCCTGGGCTTCACACCCGAGCACCAGCGGGACTTTATCGCCAGAGATCTGG
GCCCCACACTGGCCAACAGCACCCACCACAACGTGCGGCTGCTGATGCTGGACGACCAGAGACTGCTGCT
CCCCCACTGGGCCAAGGTGGTGCTGACAGACCCCGAGGCCGCCAAATACGTCCACGGGATTGCCGTGCAC
TGGTATCTGGACTTTCTGGCCCCTGCCAAGGCCACACTGGGCGAGACACACCGGCTGTTCCCCAACACCA
TGCTGTTCGCCAGCGAGGCCTGCGTGGGCAGCAAGTTCTGGGAGCAGAGCGTGCGGCTGGGCAGCTGGGA
CAGAGGCATGCAGTACAGCCACAGCATCATCACCAACCTGCTGTACCACGTCGTGGGCTGGACCGACTGG
AATCTGGCCCTGAACCCTGAGGGAGGACCCAACTGGGTCCGCAACTTCGTGGACAGCCCCATCATCGTGG
ACATCACCAAGGACACCTTCTACAAGCAGCCCATGTTCTACCACCTGGGCCACTTCAGCAAGTTCATCCC
CGAGGGCAGCCAGAGAGTGGGACTGGTCGCCAGCCAGAAGAACGATCTGGACGCCGTGGCCCTGATGCAC
CCTGATGGCAGCGCTGTGGTGGTGGTCCTGAATCGGTCCAGCAAGGACGTGCCCCTGACCATCAAGGACC
CCGCCGTGGGCTTCCTGGAAACCATCAGCCCCGGCTACTCCATCCACACCTACCTGTGGCGGAGACAAGG
AGCACCAGGCGGATCTCCAGCAGGATCTCCAACCTCTACCGAGGAAGGCACAAGCGAGTCTGCCACACCT
GAGTCTGGACCTGGCACAAGCACAGAGCCTAGCGAAGGATCTGCCCCAGGTTCTCCTGCCGGCTCTCCTA
CAAGTACAGGACCTTCTGGCGCTCCACTGTGTGGCGGAGAACTGGTGGATACCCTGCAGTTCGTGTGCGG
CGACAGAGGCTTCTACTTTAGCAGACCCGCCAGCAGAGTGTCCGCCAGATCTAGAGGAATCGTGGAAGAG
TGCTGCTTCAGAAGCTGCGATCTGGCACTGCTGGAAACCTACTGTGCCACACCAGCCAAGAGCGAGTGAT
GA
The codon-optimized human GBA having a modified signal
peptide sequence has the nucleic acid sequence of:
(SEQ ID NO: 11)
ATGGGGATTCCTATGGGCAAGTCTATGCTGGTCCTGCTGACATTTCTGGCCTTCGCTTCATGCTGTATCG
CTGCCAGACCTTGCATCCCCAAGAGCTTCGGCTACAGCAGCGTCGTGTGCGTGTGCAACGCCACCTACTG
CGACAGCTTCGACCCCCCTACCTTTCCCGCCCTGGGCACCTTCAGCAGATACGAGAGCACCCGCAGCGGC
AGACGGATGGAACTGAGCATGGGCCCCATCCAGGCCAATCACACCGGAACCGGCCTGCTGCTGACCCTGC
AGCCCGAGCAGAAATTCCAGAAAGTGAAGGGCTTCGGCGGAGCCATGACAGACGCCGCTGCCCTGAACAT
CCTGGCCCTGAGCCCCCCTGCTCAGAATCTGCTGCTCAAGAGCTACTTCAGCGAGGAAGGCATCGGCTAC
AACATCATCCGGGTGCCCATGGCTAGCTGCGACTTCAGCATCCGGACCTACACCTACGCCGACACCCCCG
ACGACTTCCAGCTGCACAACTTCAGCCTGCCCGAAGAGGACACCAAGCTGAAGATCCCCCTGATCCACAG
AGCCCTGCAGCTGGCCCAGAGGCCTGTGTCTCTGCTGGCTAGCCCTTGGACCAGCCCCACCTGGCTGAAA
ACCAACGGAGCCGTGAACGGCAAGGGCAGCCTGAAGGGCCAGCCCGGCGACATCTACCACCAGACCTGGG
CCCGGTACTTCGTGAAGTTCCTGGACGCCTATGCCGAGCACAAGCTGCAGTTCTGGGCCGTGACCGCCGA
GAATGAGCCTAGCGCTGGACTGCTGTCCGGCTACCCCTTCCAATGCCTGGGCTTCACACCCGAGCACCAG
CGGGACTTTATCGCCAGAGATCTGGGCCCCACACTGGCCAACAGCACCCACCACAACGTGCGGCTGCTGA
TGCTGGACGACCAGAGACTGCTGCTCCCCCACTGGGCCAAGGTGGTGCTGACAGACCCCGAGGCCGCCAA
ATACGTCCACGGGATTGCCGTGCACTGGTATCTGGACTTTCTGGCCCCTGCCAAGGCCACACTGGGCGAG
ACACACCGGCTGTTCCCCAACACCATGCTGTTCGCCAGCGAGGCCTGCGTGGGCAGCAAGTTCTGGGAGC
AGAGCGTGCGGCTGGGCAGCTGGGACAGAGGCATGCAGTACAGCCACAGCATCATCACCAACCTGCTGTA
CCACGTCGTGGGCTGGACCGACTGGAATCTGGCCCTGAACCCTGAGGGAGGACCCAACTGGGTCCGCAAC
TTCGTGGACAGCCCCATCATCGTGGACATCACCAAGGACACCTTCTACAAGCAGCCCATGTTCTACCACC
TGGGCCACTTCAGCAAGTTCATCCCCGAGGGCAGCCAGAGAGTGGGACTGGTCGCCAGCCAGAAGAACGA
TCTGGACGCCGTGGCCCTGATGCACCCTGATGGCAGCGCTGTGGTGGTGGTCCTGAATCGGTCCAGCAAG
GACGTGCCCCTGACCATCAAGGACCCCGCCGTGGGCTTCCTGGAAACCATCAGCCCCGGCTACTCCATCC
ACACCTACCTGTGGCGGAGACAATGA

### SCARB2 Sequences

According to the methods described herein, a patient can be administered one or more agents (e.g., one or more polynucleotides encoding SCARB2, one or more RNAi molecules that collectively increase the expression and/or activity of SCARB2 or one or more expression vectors encoding the same, a SCARB2 protein, one or more small molecule compounds that collectively increase expression and/or activity of SCARB2, and/or a pluripotent cell (e.g., an HSC, iPSC, CD34+ cell, ES cell, or myeloid progenitor cell) that expresses any one of the aforementioned polynucleotides, such as, e.g., an amino acid sequence of SEQ ID NO: 14 or 15, or a polynucleotide encoding a polypeptide having at least 85% sequence identity (e.g., 85%, 90%, 95%, 96%, 97%, 98%, 99%, or more, sequence identity) to the amino acid sequence of SEQ ID NO: 14 or 15, or a polynucleotide encoding a polypeptide that contains one or more conservative amino acid substitutions relative to SEQ ID NO: 14 or 15 (e.g., 1, 2, 3, 4, 5, 6, 7, 8, 9, or 10 or more conservative amino acid substitutions), provided that the SCARB2 variant encoded retains the therapeutic function of wild-type SCARB2.

Wild-type SCARB2 protein may have the amino acid sequence of SEQ ID NO: 14 or be a variant thereof having at least 85% (e.g., 86%, 90%, 95%, 96%, 97%, 98%, 99%, or more, sequence identity) to the amino acid sequence of SEQ ID NO: 14 (UniProt ID: Q14108.1; Isoform 1).

(SEQ ID NO: 14)
MGRCCFYTAGTLSLLLLVTSVTLLVARVFQKAVDQSIEKKIVLRNGTEAF
DSWEKPPLPVYTQFYFFNVTNPEEILRGETPRVEEVGPYTYRELRNKANI
QFGDNGTTISAVSNKAYVFERDQSVGDPKIDLIRTLNIPVLTVIEWSQVH
FLREIIEAMLKAYQQKLFVTHTVDELLWGYKDEILSLIHVFRPDISPYFG
LFYEKNGTNDGDYVFLTGEDSYLNFTKIVEWNGKTSLDWWITDKCNMING
TDGDSFHPLITKDEVLYVFPSDFCRSVYITFSDYESVQGLPAFRYKVPAE
LANTSDNAGFCIPEGNCLGSGVLNVSICKNGAPIIMSFPHFYQADERFVS
AIEGMHPNQEDHETFVDINPLTGIILKAAKRFQINIYVKKLDDFVETGDI
RTMVFPVMYLNESVHIDKETASRLKSMINTTLIITNIPYIIMALGVFFGL
VFTWLACKGQGSMDEGTADERAPLIRT

Alternatively, Wild-type SCARB2 protein may have the amino acid sequence of SEQ ID NO: 15 or be a variant thereof having at least 85% (e.g., 86%, 90%, 95%, 96%, 97%, 98%, 99%, or more, sequence identity) to the amino acid sequence of SEQ ID NO: 15 (UniProt ID: Q14108.2; Isoform 2).

(SEQ ID NO: 15)
MGRCCFYTAGTLSLLLLVTSVTLLVARVFQKAVDQSIEKKIVLRNGTEAF
DSWEKPPLPVYTQFYFFNVTNPEEILRGETPRVEEVGPYTYRSLDWWITD
KCNMINGTDGDSFHPLITKDEVLYVFPSDFCRSVYITFSDYESVQGLPAF
RYKVPAEILANTSDNAGFCIPEGNCLGSGVLNVSICKNGAPIIMSFPHFY
QADERFVSAIEGMHPNQEDHETFVDINPLTGIILKAAKRFQINIYVKKLD
DFVETGDIRTMVFPVMYLNESVHIDKETASRLKSMINTTLIITNIPYIIM
ALGVFFGLVFTWLACKGQGSMDEGTADERAPLIRT

SCARB2 may be encoded by a polynucleotide having the nucleic acid sequence of SEQ ID NO: 16 or a variant thereof having at least 85% (e.g., 86%, 90%, 95%, 96%, 97%, 98%, 99%, or more, sequence identity) to the amino acid sequence of SEQ ID NO: 16 (NCBI Accession No.: NM_005506.3; Isoform 1).

(SEQ ID NO: 16)
GCAGTCGCCGGGGGTTGCCTCGCGGGGTTGCGGCGAGCCCGGCCCGCGAA
CGTCACGTCCCTGCGCGCTCCCTGCACTCTCCCGAGCTGCGCTAGGCGGG
CGCCACGGCTGCCCGGCGAAGGAAACCGAAACCGAGTCCGGGCCCGTCCC
TCCGCGGCCCCATCCGCCCGGTGCACCCGGGGCCGCGCTCGCCAGGCCGC
GGAGCCCAGAGCTGCGCGCACGAACCGTGCGCCGGGAGGGCGTGGGCGTG
GCGCCGAAGGGTCCCGGGTCTTCGACGCCTCTGCGGCGGCTCCTCCCTCC
TTGCAGTTGGATCCCTGGCGGGTGCGGCCCGGCCCGGCCCGTGAGCGGCG
CACAGAATGGGCCGATGCTGCTTCTACACGGCGGGGACGTTGTCCCTGCT
CCTGCTGGTGACCAGCGTCACGCTGCTGGTGGCCCGGGTCTTCCAGAAGG
CTGTAGACCAGAGTATCGAGAAGAAAATTGTGTTAAGGAATGGTACTGAG
GCATTTGACTCCTGGGAGAAGCCCCCTCTGCCTGTGTATACTCAGTTCTA
TTTCTTCAATGTCACCAATCCAGAGGAGATCCTCAGAGGGGAGACCCCTC
GGGTGGAAGAAGTGGGGCCATACACCTACAGGGAACTCAGAAACAAAGCA
AATATTCAATTTGGAGATAATGGAACAACAATATCTGCTGTTAGCAACAA
GGCCTATGTTTTTGAACGAGACCAATCTGTTGGAGACCCTAAAATTGACT
TAATTAGAACATTAAATATTCCTGTATTGACTGTCATAGAGTGGTCCCAG
GTGCACTTCCTCAGGGAGATCATCGAGGCCATGTTGAAAGCCTATCAGCA
GAAGCTCTTTGTGACTCACACAGTTGACGAATTGCTCTGGGGCTACAAAG
ATGAAATCTTGTCCCTTATCCATGTTTTCAGGCCCGATATCTCTCCCTAT
TTTGGCCTATTCTATGAGAAAAATGGGACTAATGATGGAGACTATGTTTT
TCTAACTGGAGAAGACAGTTACCTTAACTTTACAAAAATTGTGGAATGGA
ATGGGAAAACGTCACTTGACTGGTGGATAACAGACAAGTGCAATATGATT
AATGGAACAGATGGAGATTCTTTTCACCCACTAATAACCAAAGATGAGGT
CCTTTATGTCTTCCCATCTGACTTTTGCAGGTCAGTGTATATTACTTTCA
GTGACTATGAGAGTGTACAGGGACTGCCTGCCTTTCGGTATAAAGTTCCT
GCAGAAATATTAGCCAATACGTCAGACAATGCCGGCTTCTGTATACCTGA
GGGAAACTGCCTGGGCTCAGGAGTTCTGAATGTCAGCATCTGCAAGAATG
GTGCACCCATCATTATGTCTTTCCCACACTTTTACCAAGCAGATGAGAGG
TTTGTTTCTGCCATAGAAGGCATGCACCCAAATCAGGAAGACCATGAGAC
ATTTGTGGACATTAATCCTTTGACTGGAATAATCCTAAAAGCAGCCAAGA
GGTTCCAAATCAACATTTATGTCAAAAAATTAGATGACTTTGTTGAAACG
GGAGACATTAGAACCATGGTTTTCCCAGTGATGTACCTCAATGAGAGTGT
TCACATTGATAAAGAGACGGCGAGTCGACTGAAGTCTATGATTAACACTA
CTTTGATCATCACCAACATACCCTACATCATCATGGCGCTGGGTGTGTTC
TTTGGTTTGGTTTTTACCTGGCTTGCATGCAAAGGACAGGGATCCATGGA
TGAGGGAACAGCGGATGAAAGAGCACCCCTCATTCGAACCTAAACATTGC
CTTTGCTTGGTGAAGAAACTGTGTGAGCTGTCCTGACCTGGACGATGACG
TGGGGAAACCCTCCACCTCCTTGCAGGCTTGTTGCCTGTTGAAAGAAGGA
AAAAGACACGGCGCTGGCAAGTGATAGGAACATTCTGGCCAGAGGTTAAA
GAGCAGGCTGACATGGCTGGCCATTAAGCTTTATAAAATCATGTGGGCTC
TGAAATTGTTCTTTTATGTGTCTAGCAAGTATTTAATAAACCCTTGTATA
GTAATTTTGTTGTTGTTGGGTGCTGGTAGCTCCAGAATTTTGTGACCACT
ATTGTGGGTAAAATGTCTCTGCATCACTTGTTAATGCTACTGGTCTAACT
TCATTCAGTATGCTTCATTCACCGAACTTTGTGCTCAAAATGCGTATATA
CCATTTTATGTTGTATTCCTCCATTTCACTTGCAAAACAGAAGTAAATAA
GAGTTCGGGACCCAGGGTAAAATGGTAGCTTCATCCAATATATCATTCAA
ATGCATCTGATTTCTAAAACATATTACATTTTATGCTGATCTTCAGTTCA
TAATTCTTCCAGGAAAACTCAGTCTTCCAACTGCAATAAAATACTGGGTA
GAATCAAATGGGAAAGGGGTTGGGTGGGGCAATACCCATGAGTTGATAGT
GATAAGCTCCTAAGGATTTTTAACTTGTACTTTTGTGAACGAAGAGAATG
CATAAATAATGTTGGTGAGGATAAAGTACAGATATTTCATGTAGAATTAA
TTGCTAGTTATGATGCTTGTGGATAGTTAACTGTTTTTTTTTTAGTCAAA
ATGATCATGCTACGAAAAGATGCTTCTGAGAGAATGTAATGAGTAACTGA
TTTTTCTTCCTGAGTCGCCCTTGCCAAATATGTTACTGTATTAATTAATC
TAATATTGAGTGATTATTTGTAAAATTATGAATATGGGAAATCCATCTAT
CTACAGCCTAAGTTACACATAAGTTTCAGAAAGTCTGATTAGACTAAAGA
GATATTTCTTCTGGGACAGCCGCCTTCTTGGTAATTTTGAAGTTCTTTTT
ACAAGTTCCTTCCTCAGTTTCAGTTCTTTCCAGTGTTTTGTAGCTCACTG
TCACTCACTGAATAGAGAAACGTGTGCCCTATACTTCCTGTGACAATCAT
TTTGCTGACAGAATGATGGATGTTTAAAATATTGCACAAAGTACTTTAAA
GAAAGGTCTGTTAGGACCAGAAGCAGAGACACCACTTTTCAAAGGACTTC
TTGGTTTCAGCATAACCTAAGACAGGGAATTGGGAGCCATCATATGTCAC
AGTGTTCAGAATTCAAGCATATTTAAGGGCATTTTCTTTGATTCTCAAAG
TTCAGCATTCATTTTGAATTGAGAAGCCTATACATTTAGCTGACAAAGTG
CTTATAGAATTTCTTAACAACTGAACCATTCAAAAGGATTTTTTTTGTTT
AAAACTGGATTTCAATGTAAGCAAATGAAGAAAAAAATATAGATTTCATT
TCCATAGCTTCTTATCCCTGTATTGAGGTAATAAATTGTTTTACTGACAA
TTTTTCCTTTTTCTACACTAAAACAATATGTGATATATTTCCCCTCTTGA
AGAGGCAATTCATTAAACTCTCAAATTTTCTATAGAATCAAGATAGAACC
TTTAGATACTCCAACTCACCAAAATGTAAAAAAACTAACAAAAATATTTG
GTCTTCAATAATGCTAAATATCTACATTTTTAGAATTTATCAACATTTAA
CTAGATAATTGGGCATGTCTTAATTATGCATGTACTTATCCATACTAATA
AAATTGACAATGCTAGTGCATACTTATTGGTTTAGTCCTATTATCAGGAT
ATAATCATCTGTGAGGAGGATATTTTAAATACTGTAAATGATAACAGTTA
ATGATATACACATTTAGACTGAGTTGCACACTGGCAGGGAGACCAAAAAC
ATTACTTCCATACTTGTGTCATGATTCTTTTTTTTTTGAGAGAGTCTCAC
TCTGTCGCCAGGCTGGAGTACAGTGGCATGATCTCGGCTCACTGCAACCT
CTGCCTCCCGGGTTCAAGCAATTCTCCTGCCTCAGCCACCCAAGTAGCTG
GGACTACAGGTGCGTGCCACCACGCCCAGCTAAATTTTGTATTTTTAGTG
GAGACGGGGTTTCACCATGTTGGCCAGGATGGTCTCAATCTCCTGACCCT
GCGATCTGCCCACCTCAGCCTCCCAAAGTGCTGGGATTACAGGCGTAAGC
CACCGGGCCTGGCCTGTTTTATGATTCTTAATAGTTACTTGGTTTAAATC
ACATTTGATACTATCCTTCTGAAAAGTCTGAGACAGATCTACAAACTACA
GTCAAAATTATAGATTAAGAGGAATGAATGCACCTATTTGGCTTTAAGTT
GAAGATGAATTATTTCTCATGCTCATTTTCTTGCGGCAGTTATCTTAGAA
AGACCCCCAAAGGCTTTGTGATTGTAAGCACTGTCATGATCACAGAATGC
AAGCTTCTGGTACCATGATCCTCAACTTAGAGAGGAAGAAACCAAGACAG
AGAGCTTAACTCACTTCTCTCAGGGAAAATTAGGAGTTGAGCACAGGACA
GGAAATGGGCTTTGCCACTTTTAGCTCCAGGCTTTTCTAACCAGACTTGA
TTTCCTCATGTTCTAGAAAGATCACTAATGGTCAAGTGGAACAAGCACTA
CACGACTAACCCCTATTGGGGTTTTTAACTTAAGGGAGGCTAATTTTTAA
TTTAAACTGCTCGAGATATGAGTTCTGCAAAAGGTGGTCCGCATCCTTGG
CCCTCTGGACATTATCACTAAATTGCTTGTGCCTGTTAACAAGAATACTG
ACCAGAATGCTCTTCATGTAGCTTATACAGTTGGTTCACTTCATGCGGTT
CTTGACATGTTTATTTCTACCCTTAATGCAATGAAATGTTTCATTAATAA
AAAACCACTTTATATAAAAAAAAAAAAAAA

Alternatively, SCARB2 may be encoded by a polynucleotide having the nucleic acid sequence of SEQ ID NO: 17 or a variant thereof having at least 85% (e.g., 86%, 90%, 95%, 96%, 97%, 98%, 99%, or more, sequence identity) to the amino acid sequence of SEQ ID NO: 17 (NCBI Accession No.: NM_001204255.1; Isoform 2).

(SEQ ID NO: 17)
GCAGTCGCCGGCGGTTGCCTCGCGGGGTTGCGGCGAGCCCGGCCCGCGAA
CGTCACGTCCCTGCGCGCTCCCTGCACTCTCCCGAGCTGCGCTAGGCGGG
CGCCACGGCTGCCCGGCGAAGGAAACCGAAACCGAGTCCGGGCCCGTCCC
TCCGCGGCCCCATCCGCCCGGTGCACCCGGGGCCGCGCTCGCCAGGCCGC
GGAGCCCAGAGCTGCGCGCACGAACCGTGCGCCGGGAGGGCGTGGGCGTG
GCGCCGAAGGGTCCCGGGTCTTCGACGCCTCTGCGGCGGCTCCTCCCTCC
TTGCAGTTGGATCCCTGGCGGGTGCGGCCCGGCCCGGCCCGTGAGCGGCG
CACAGAATGGGCCGATGCTGCTTCTACACGGCGGGGACGTTGTCCCTGCT
CCTGCTGGTGACCAGCGTCACGCTGCTGGTGGCCCGGGTCTTCCAGAAGG
CTGTAGACCAGAGTATCGAGAAGAAAATTGTGTTAAGGAATGGTACTGAG
GCATTTGACTCCTGGGAGAAGCCCCCTCTGCCTGTGTATACTCAGTTCTA
TTTCTTCAATGTCACCAATCCAGAGGAGATCCTCAGAGGGGAGACCCCTC
GGGTGGAAGAAGTGGGGCCATACACCTACAGGTCACTTGACTGGTGGATA
ACAGACAAGTGCAATATGATTAATGGAACAGATGGAGATTCTTTTCACCC
ACTAATAACCAAAGATGAGGTCCTTTATGTCTTCCCATCTGACTTTTGCA
GGTCAGTGTATATTACTTTCAGTGACTATGAGAGTGTACAGGGACTGCCT
GCCTTTCGGTATAAAGTTCCTGCAGAAATATTAGCCAATACGTCAGACAA
TGCCGGCTTCTGTATACCTGAGGGAAACTGCCTGGGCTCAGGAGTTCTGA
ATGTCAGCATCTGCAAGAATGGTGCACCCATCATTATGTCTTTCCCACAC
TTTTACCAAGCAGATGAGAGGTTTGTTTCTGCCATAGAAGGCATGCACCC
AAATCAGGAAGACCATGAGACATTTGTGGACATTAATCCTTTGACTGGAA
TAATCCTAAAAGCAGCCAAGAGGTTCCAAATCAACATTTATGTCAAAAAA
TTAGATGACTTTGTTGAAACGGGAGACATTAGAACCATGGTTTTCCCAGT
GATGTACCTCAATGAGAGTGTTCACATTGATAAAGAGACGGCGAGTCGAC
TGAAGTCTATGATTAACACTACTTTGATCATCACCAACATACCCTACATC
ATCATGGCGCTGGGTGTGTTCTTTGGTTTGGTTTTTACCTGGCTTGCATG
CAAAGGACAGGGATCCATGGATGAGGGAACAGCGGATGAAAGAGCACCCC
TCATTCGAACCTAAACATTGCCTTTGCTTGGTGAAGAAACTGTGTGAGCT
GTCCTGACCTGGACGATGACGTGGGGAAACCCTCCACCTCCTTGCAGGCT
TGTTGCCTGTTGAAAGAAGGAAAAAGACACGGCGCTGGCAAGTGATAGGA
ACATTCTGGCCAGAGGTTAAAGAGCAGGCTGACATGGCTGGCCATTAAGC
TTTATAAAATCATGTGGGCTCTGAAATTGTTCTTTTATGTGTCTAGCAAG
TATTTAATAAACCCTTGTATAGTAATTTTGTTGTTGTTGGGTGCTGGTAG
CTCCAGAATTTTGTGACCACTATTGTGGGTAAAATGTCTCTGCATCACTT
GTTAATGCTACTGGTCTAACTTCATTCAGTATGCTTCATTCACCGAACTT
TGTGCTCAAAATGCGTATATACCATTTTATGTTGTATTCCTCCATTTCAC
TTGCAAAACAGAAGTAAATAAGAGTTCGGGACCCAGGGTAAAATGGTAGC
TTCATCCAATATATCATTCAAATGCATCTGATTTCTAAAACATATTACAT
TTTATGCTGATCTTCAGTTCATAATTCTTCCAGGAAAACTCAGTCTTCCA
ACTGCAATAAAATACTGGGTAGAATCAAATGGGAAAGGGGTTGGGTGGGG
CAATACCCATGAGTTGATAGTGATAAGCTCCTAAGGATTTTTAACTTGTA
CTTTTGTGAACGAAGAGAATGCATAAATAATGTTGGTGAGGATAAAGTAC
AGATATTTCATGTAGAATTAATTGCTAGTTATGATGCTTGTGGATAGTTA
ACTGTTTTTTTTTTAGTCAAAATGATCATGCTACGAAAAGATGCTTCTGA
GAGAATGTAATGAGTAACTGATTTTTCTTCCTGAGTCGCCCTTGCCAAAT
ATGTTACTGTATTAATTAATCTAATATTGAGTGATTATTTGTAAAATTAT
GAATATGGGAAATCCATCTATCTACAGCCTAAGTTACACATAAGTTTCAG
AAAGTCTGATTAGACTAAAGAGATATTTCTTCTGGGACAGCCGCCTTCTT
GGTAATTTTGAAGTTCTTTTTACAAGTTCCTTCCTCAGTTTCAGTTCTTT
CCAGTGTTTTGTAGCTCACTGTCACTCACTGAATAGAGAAACGTGTGCCC
TATACTTCCTGTGACAATCATTTTGCTGACAGAATGATGGATGTTTAAAA
TATTGCACAAAGTACTTTAAAGAAAGGTCTGTTAGGACCAGAAGCAGAGA
CACCACTTTTCAAAGGACTTCTTGGTTTCAGCATAACCTAAGACAGGGAA
TTGGGAGCCATCATATGTCACAGTGTTCAGAATTCAAGCATATTTAAGGG
CATTTTCTTTGATTCTCAAAGTTCAGCATTCATTTTGAATTGAGAAGCCT
ATACATTTAGCTGACAAAGTGCTTATAGAATTTCTTAACAACTGAACCAT
TCAAAAGGATTTTTTTTGTTTAAAACTGGATTTCAATGTAAGCAAATGAA
GAAAAAAATATAGATTTCATTTCCATAGCTTCTTATCCCTGTATTGAGGT
AATAAATTGTTTTACTGACAATTTTTCCTTTTTCTACACTAAAACAATAT
GTGATATATTTCCCCTCTTGAAGAGGCAATTCATTAAACTCTCAAATTTT
CTATAGAATCAAGATAGAACCTTTAGATACTCCAACTCACCAAAATGTAA
AAAAACTAACAAAAATATTTGGTCTTCAATAATGCTAAATATCTACATTT
TTAGAATTTATCAACATTTAACTAGATAATTGGGCATGTCTTAATTATGC
ATGTACTTATCCATACTAATAAAATTGACAATGCTAGTGCATACTTATTG
GTTTAGTCCTATTATCAGGATATAATCATCTGTGAGGAGGATATTTTAAA
TACTGTAAATGATAACAGTTAATGATATACACATTTAGACTGAGTTGCAC
ACTGGCAGGGAGACCAAAAACATTACTTCCATACTTGTGTCATGATTCTT
TTTTTTTTGAGAGAGTCTCACTCTGTCGCCAGGCTGGAGTACAGTGGCAT
GATCTCGGCTCACTGCAACCTCTGCCTCCCGGGTTCAAGCAATTCTCCTG
CCTCAGCCACCCAAGTAGCTGGGACTACAGGTGCGTGCCACCACGCCCAG
CTAAATTTTGTATTTTTAGTGGAGACGGGGTTTCACCATGTTGGCCAGGA
TGGTCTCAATCTCCTGACCCTGCGATCTGCCCACCTCAGCCTCCCAAAGT
GCTGGGATTACAGGCGTAAGCCACCGGGCCTGGCCTGTTTTATGATTCTT
AATAGTTACTTGGTTTAAATCACATTTGATACTATCCTTCTGAAAAGTCT
GAGACAGATCTACAAACTACAGTCAAAATTATAGATTAAGAGGAATGAAT
GCACCTATTTGGCTTTAAGTTGAAGATGAATTATTTCTCATGCTCATTTT
CTTGCGGCAGTTATCTTAGAAAGACCCCCAAAGGCTTTGTGATTGTAAGC
ACTGTCATGATCACAGAATGCAAGCTTCTGGTACCATGATCCTCAACTTA
GAGAGGAAGAAACCAAGACAGAGAGCTTAACTCACTTCTCTCAGGGAAAA
TTAGGAGTTGAGCACAGGACAGGAAATGGGCTTTGCCACTTTTAGCTCCA
GGCTTTTCTAACCAGACTTGATTTCCTCATGTTCTAGAAAGATCACTAAT
GGTCAAGTGGAACAAGCACTACACGACTAACCCCTATTGGGGTTTTTAAC
TTAAGGGAGGCTAATTTTTAATTTAAACTGCTCGAGATATGAGTTCTGCA
AAAGGTGGTCCGCATCCTTGGCCCTCTGGACATTATCACTAAATTGCTTG
TGCCTGTTAACAAGAATACTGACCAGAATGCTCTTCATGTAGCTTATACA
GTTGGTTCACTTCATGCGGTTCTTGACATGTTTATTTCTACCCTTAATGC
AATGAAATGTTTCATTAATAAAAAACCACTTTATATAAAA

In some embodiments, the SCARB2 is a GBA-binding domain of SCARB2, such as, e.g., a GBA-binding domain of SCARB2 having an amino acid sequence of LREIIEAMLKAYQQKL (SEQ ID NO: 18; (Reczek et al. Cell 131:770-83 (2007)), which include residues 150-167 of the SCARB2 protein. In some embodiments, the GBA-binding domain of SCARB2 is a variant having at least 70% (e.g., at least 71%, 75%, 80%, 85%, 90%, 91%, 92%, 93%, 94%, 95%, 96%, 97%, 98%, 99% or more) sequence identity to the amino acid sequence of SEQ ID NO: 18.

In some embodiments, the GBA-binding domain of SCARB2 has an amino acid sequence of LREIIEAMLKAYQQKLFVTHTVDELLWGYKDEILSLIHVF (SEQ ID NO: 19), which include residues 152-191 of the SCARB2 protein. In some embodiments, the GBA-binding domain of SCARB2 is a variant having at least 70% (e.g., at least 71%, 75%, 80%, 85%, 90%, 91%, 92%, 93%, 94%, 95%, 96%, 97%, 98%, 99% or more) sequence identity to the amino acid sequence of SEQ ID NO: 19 (Zunke et al. PNAS 113:3791-6 (2016)).

### Therapeutic Agents

Agents that elevate the expression and/or activity level of GBA and SCARB2 proteins that may be used in conjunction with the compositions and methods of the disclosure include nucleic acids that encode the GBA and/or SCARB2 protein (e.g., nucleic acids capable of expression in macrophages or microglia). Such nucleic acid molecules may be provided to a patient (e.g., a Gaucher disease patient) in the form, for example, of a population of cells, such as a population of cells (e.g., pluripotent cells, ESCs, iPSCs, multipotent cells, CD34+ cells, HSCs, MPCs, BLPCs, monocytes, macrophages, microglial progenitor cells, or microglia) that contain the nucleic acid molecules. Such cells may be modified ex vivo so as to express the nucleic acid molecule(s) of interest, for example, using transfection and transduction methods described herein. Additionally or alternatively, nucleic acid molecules encoding one or more of the proteins of interest may be provided to the patient in the form of one or more viral vectors that collectively encode the one or more proteins. Exemplary viral vectors that may be used in conjunction with the compositions and methods of the disclosure include Retroviridae family viral vectors, such as a lentivirus, alpharetrovirus, or gammaretrovirus, among others described herein. In some embodiments, the nucleic acid molecule(s) are administered directly to the patient. Additional agents that may be provided to a patient for the purpose of augmenting the level of GBA and/or SCARB2 include interfering RNA molecules, such as siRNA, shRNA, and miRNA molecules, as well as small molecule agents that modulate the expression of one or more of the above proteins, in addition to the one or more of the above proteins themselves.

### Therapeutic Cells

Cells that may be used in conjunction with the compositions and methods described herein include cells that are capable of undergoing further differentiation. For example, one type of cell that can be used in conjunction with the compositions and methods described herein is a pluripotent cell (e.g., a CD34+ cell). A pluripotent cell is a cell that possesses the ability to develop into more than one differentiated cell type. Examples of pluripotent cells are embryonic stem cells (ES cells) and induced pluripotent stem cells (iPS cells or iPSCs). ES cells and PS cells have the ability to differentiate into cells of the ectoderm, which gives rise to the skin and nervous system, endoderm, which forms the gastrointestinal and respiratory tracts, endocrine glands, liver, and pancreas, and mesoderm, which forms bone, cartilage, muscles, connective tissue, and most of the circulatory system.

Cells that may be used in conjunction with the compositions and methods described herein include hematopoietic stem cells and myeloid progenitor cells. Hematopoietic stem cells (HSCs) are immature blood cells that have the capacity to self-renew and to differentiate into mature blood cells including diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Human HSCs are CD34+. In addition, HSCs also refer to long term repopulating HSC (LT-HSC) and short-term repopulating HSC (ST-HSC). Any of these HSCs can be used in conjunction with the compositions and methods described herein.

HSCs can differentiate into myeloid progenitor cells, which are also CD34+. Myeloid progenitors can further differentiate into granulocytes (e.g., promyelocytes, neutrophils, eosinophils, and basophils), erythrocytes (e.g., reticulocytes, erythrocytes), thrombocytes (e.g., megakaryoblasts, platelet producing megakaryocytes, and platelets), monocytes (e.g., monocytes and macrophages), dendritic cells, and microglia. Common myeloid progenitors can be characterized by cell surface molecules and are known to be lin−, SCA1−, c-kit+, CD34+, and CD16/32^{mid}.

HSCs and myeloid progenitors can be obtained from blood products. A blood product is a product obtained from the body or an organ of the body containing cells of hematopoietic origin. Such sources include unfractionated bone marrow, umbilical cord, placenta, peripheral blood, or mobilized-peripheral blood. All of the aforementioned crude or unfractionated blood products can be enriched for cells having HSC or myeloid progenitor cell characteristics in a number of ways. For example, the more mature, differentiated cells can be selected against based on cell surface molecules they express. The blood product may be fractionated by positively selecting for CD34+ cells, which include a subpopulation of hematopoietic stem cells capable of self-renewal, multi-potency, and that can be re-introduced into a transplant recipient whereupon they home to the hematopoietic stem cell niche and reestablish productive and sustained hematopoiesis. Such selection is accomplished using, for example, commercially available magnetic anti-CD34 beads (Dynal, Lake Success, NY). Myeloid progenitor cells can also be isolated based on the markers they express. Unfractionated blood products can be obtained directly from a donor or retrieved from cryopreservative storage. HSCs and myeloid progenitor cells can also be obtained from by differentiation of ES cells, iPS cells or other reprogrammed mature cell types.

Cells that may be used in conjunction with the compositions and methods described herein include allogeneic cells or autologous cells. All of the aforementioned cell types are capable of differentiating into microglia. Cells may also differentiate into microglial progenitors or microglial stem cells. Differentiation may occur ex vivo or in vivo. Methods for ex vivo differentiation of human ES cells and iPS cells are known by those of skill in the art and are described in Muffat et al., Nature Medicine 22:1358-1367 (2016) and Pandya et al., Nature Neuroscience 20:753-9 (2017), the disclosures of which are incorporated herein by reference as they pertain to methods of differentiating pluripotent cells into microglia.

### Microglia

Cells that may be used in conjunction with the compositions and methods described herein include those that are capable of differentiating into microglial cells. Microglia are myeloid-derived cells that serve as the immune cells, or resident macrophages, of the central nervous system. Microglia are highly similar to macrophages, both genetically and functionally, and share the ability to shift dynamically between pro-inflammatory and anti-inflammatory states. Microglia can be made to shift between pro-inflammatory and anti-inflammatory states by extracellular signals, e.g., signals from neighboring neurons or astrocytes, cell debris, toxins, infection, ischemia, and traumatic injury, among others. Activated microglia are often observed in the diseased brain, particularly in diseases involving neuroinflammation, such as Gaucher disease (Vitner et al. Brain 135:1724-35 (2012)). Activated microglial phenotypes have also been observed in murine models of Gaucher disease (Vitner et al. Brain 135:1724-35 (2012)). It is unclear whether Pro-inflammatory microglia are a cause or consequence of neuroinflammation, but once microglia are activated, they can secrete pro-inflammatory cytokines, e.g., TNF-α, IL-1β, and IL-6, chemokines, and nitric oxide, which can lead to sustained inflammation, neuronal damage, and further pro-inflammatory activation microglia.

### Expression of GBA and SCARB2 in Host Cells

GBA activity is reduced in patients with Gaucher disease, and Gaucher disease brains contain activated pro-inflammatory microglia. The compositions and methods described herein target these dysfunctions by administering cells expressing a transgene encoding GBA (e.g., non-secreted GBA or secreted GBA) and/or SCARB2. In order to utilize these agents for therapeutic application in the treatment of Gaucher disease, these agents can be directed to the interior of the cell, and in particular examples, to particular organelles (e.g., lysosomes). A wide array of methods has been established for the delivery of such proteins to mammalian cells and for the stable expression of genes encoding such proteins in mammalian cells.

Polynucleotides Encoding GBA and/or SCARB2

One platform that can be used to achieve therapeutically effective intracellular concentrations of GBA and SCARB2 in mammalian cells is via the stable expression of genes encoding these proteins (e.g., by integration into the nuclear or mitochondrial genome of a mammalian cell). These genes are polynucleotides that encode the primary amino acid sequence of the corresponding protein. In order to introduce such exogenous genes into a mammalian cell, these genes can be incorporated into a vector. Vectors can be introduced into a cell by a variety of methods, including transformation, transfection, direct uptake, projectile bombardment, and by encapsulation of the vector in liposomes. Examples of suitable methods of transfecting or transforming cells are calcium phosphate precipitation, electroporation, microinjection, infection, lipofection, and direct uptake. Such methods are described in more detail, for example, in Green et al., Molecular Cloning: A Laboratory Manual, Fourth Edition (Cold Spring Harbor University Press, New York (2014)); and Ausubel et al., Current Protocols in Molecular Biology (John Wiley & Sons, New York (2015)), the disclosures of each of which are incorporated herein by reference.

GBA and SCARB2 can also be introduced into a mammalian cell by targeting a vector containing a gene(s) encoding such proteins to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding GBA and/or SCARB2 by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith et al., Mol. Sys. Biol. 3:73 (2007), the disclosure of which is incorporated herein by reference.

Polynucleotides suitable for use with the compositions and methods described herein also include those that encode GBA and/or SCARB2 downstream of a mammalian promoter. Promoters that are useful for the expression of GBA and/or SCARB2 in mammalian cells include, e.g., elongation factor 1-alpha (EF1α) promoter, phosphoglycerate kinase 1 (PGK) promoter, CD68 molecule (CD68) promoter (see Dahl et al., Molecular Therapy 23:835 (2015), incorporated herein by reference as it pertains to the use of PGK and CD68 promoters to express GBA), C-X3-C motif chemokine receptor 1 (CX3CR1) promoter, integrin subunit alpha M (ITGAM) promoter, allograft inflammatory factor 1 (AIF1) promoter, purinergic receptor P2Y12 (P2Y12) promoter, transmembrane protein 119 (TMEM119) promoter, and colony stimulating factor 1 receptor (CSF1R) promoter. Alternatively, promoters derived from viral genomes can also be used for the stable expression of these agents in mammalian cells. Examples of functional viral promoters that can be used to promote mammalian expression of these agents are adenovirus late promoter, vaccinia virus 7.5K promoter, simian virus 40 (SV40) promoter, cytomegalovirus promoter, tk promoter of herpes simplex virus (HSV), mouse mammary tumor virus (MMTV) promoter, long terminal repeat (LTR) promoter of human immunodeficiency virus (HIV), promoter of moloney virus, Epstein barr virus (EBV), Rous sarcoma virus (RSV), and the cytomegalovirus (CMV) promoter.

In some embodiments, the promoter is a synthetic promoter. In some embodiments, the synthetic promoter is an MND promoter. In some embodiments, the MND promoter includes a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 20. In some embodiments, the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO: 20. In some embodiments, the MND promoter includes a polynucleotide having at least 95% (e.g., at least 96%, 97%, 98%, 99%, or more) sequence identity to the nucleic acid sequence of SEQ ID NO: 20. In some embodiments, the MND promoter includes a polynucleotide having at least 98% (e.g., at least 99%, or more) sequence identity to the nucleic acid sequence of SEQ ID NO: 20. In some embodiments, the MND promoter includes a polynucleotide having at least 99% sequence identity to the nucleic acid sequence of SEQ ID NO: 20. In some embodiments, the MND promoter includes a polynucleotide having the nucleic acid sequence of SEQ ID NO: 20. In some embodiments, the MND promoter includes a polynucleotide having at least 85% (e.g., at least 90%, 91%, 92%, 93%, 94%, 95%, 96%, 97%, 98%, 99%, or more) sequence identity to the nucleic acid sequence of SEQ ID NO: 21. In some embodiments, the MND promoter includes a polynucleotide having at least 90% (e.g., at least 91%, 92%, 93%, 94%, 95%, 96%, 97%, 98%, 99%, or more) sequence identity to the nucleic acid sequence of SEQ ID NO: 21. In some embodiments, the MND promoter includes a polynucleotide having at least 95% (e.g., at least 96%, 97%, 98%, 99%, or more) sequence identity to the nucleic acid sequence of SEQ ID NO: 21. In some embodiments, the MND promoter includes a polynucleotide having at least 98% (e.g., at least 99%, or more) sequence identity to the nucleic acid sequence of SEQ ID NO: 21. In some embodiments, the MND promoter includes a polynucleotide having at least 99% sequence identity to the nucleic acid sequence of SEQ ID NO: 21. In some embodiments, the MND promoter includes a polynucleotide having the nucleic acid sequence of SEQ ID NO: 21.

MND promoter sequence 1
(SEQ ID NO: 20)
GATCAAGGTTAGGAACAGAGAGACAGGAGAATATGGGCCAAACAGGATAT
CTGTGGTAAGCAGTTCCTGCCCCGGCTCAGGGCCAAGAACAGTTGGAACA
GCAGAATATGGGCCAAACAGGATATCTGTGGTAAGCAGTTCCTGCCCCGG
CTCAGGGCCAAGAACAGATGGTCCCCAGATGCGGTCCCGCCCTCAGCAGT
TTCTAGAGAACCATCAGATGTTTCCAGGGTGCCCCAAGGACCTGAAATGA
CCCTGTGCCTTATTTGAACTAACCAATCAGTTCGCTTCTCGCTTCTGTTC
GCGCGCTTCTGCTCCCCGAGCTCAATAAAAGAGCCCACAACCCCTCACTC
GGCGCGCCAGTCCTCCGATAGACTGCGTCGCCCGG
MND promoter sequence 2
(SEQ ID NO: 21)
TTATTTAGTCTCCAGAAAAAGGGGGGAATGAAAGACCCCACCTGTAGGTT
TGGCAAGCTAGGATCAAGGTTAGGAACAGAGAGACAGCAGAATATGGGCC
AAACAGGATATCTGTGGTAAGCAGTTCCTGCCCCGGCTCAGGGCCAAGAA
CAGTTGGAACAGCAGAATATGGGCCAAACAGGATATCTGTGGTAAGCAGT
TCCTGCCCCGGCTCAGGGCCAAGAACAGATGGTCCCCAGATGCGGTCCCG
CCCTCAGCAGTTTCTAGAGAACCATCAGATGTTTCCAGGGTGCCCCAAGG
ACCTGAAATGACCCTGTGCCTTATTTGAACTAACCAATCAGTTCGCTTCT
CGCTTCTGTTCGCGCGCTTCTGCTCCCCGAGCTCAATAAAAGAGCCCA

Once a polynucleotide encoding GBA and/or SCARB2 has been incorporated into the nuclear DNA of a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms are tetracycline and doxycycline. These reagents are commercially available (Life Technologies, Carlsbad, CA) and can be administered to a mammalian cell in order to promote gene expression according to established protocols.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode GBA and/or SCARB2 and additionally include a mammalian enhancer sequence. Many enhancer sequences are now known from mammalian genes, and examples are enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982). An enhancer may be spliced into a vector containing a polynucleotide encoding GBA and/or SCARB2, for example, at a position 5′ or 3′ to this gene. In a preferred orientation, the enhancer is positioned at the 5′ side of the promoter, which in turn is located 5′ relative to the polynucleotide encoding GBA and/or SCARB2.

### Cell-Specific Gene Expression

Interfering RNA (RNAi) are widely used to knock down the expression of endogenous genes by delivering small interfering RNA (siRNA) into cells triggering the degradation of complementary mRNA. An additional application is to utilize the diversity of endogenous micro RNAs (miRNA) to negatively regulate the expression of exogenously introduced transgenes tagged with artificial miRNA target sequences. These miRNA target tagged transgenes can be negatively regulated according to the activity of a given miRNA which can be tissue, lineage, activation, or differentiation stage specific. These artificial miRNA target sequences (miRTs) can be recognized as targets by a specific miRNA thus inducing post-transcriptional gene silencing. While robust transgene expression in targeted cells can have beneficial therapeutic results, off target expression, such as the ectopic or non-regulated transgene expression in HSPCs or other progenitor cells, can have cytotoxic effects, which can result in counter-selection of transgene-containing cells leading to altered cellular behavior and reduced therapeutic efficacy. The incorporation of miRTs for miRNAs widely expressed in HSPCs and progenitors, but absent in cells of the myeloid lineage can allow for repressed transgene expression in HSPCs and other progenitor cells allowing for silent, long-term reservoir transgene-containing hematopoietic progeny, while allowing for robust transgene expression in differentiated, mature target cells. miR-126 is highly expressed in HSPCs, other progenitor cells, and cells of the erythroid lineage, but absent from those of the myeloid lineage (e.g., macrophages and microglia)(Gentner et al., Science Translational Medicine. 2:58ra34 (2010)). A miR-126 targeting sequence, for example, incorporated within a transgene would allow for targeted expression of the transgene in cells of the myeloid lineage and repressed expression in HSPCs and other progenitor cells, thus minimizing off-target cytotoxic effects. In some embodiments, the transgene encoding GBA and/or SCARB2 may include a miR-126 targeting sequence.

### Signal Peptides

Polynucleotides encoding GBA and/or SCARB2 may include one or more polynucleotides encoding a signal peptide. Signal peptides may have amino acid sequence of 5-30 residues in length, and may be located upstream of (e.g., 5′ to) a polynucleotide encoding GBA and/or SCARB2. These signal peptides allow for recognition of the nascent GBA and/or SCARB2 polypeptides during synthesis by signal recognition particles resulting transport across the membrane of the rough endoplasmic reticulum, as well as glycosylation for transport into lysosomes. Exemplary signal peptides for lysosomal transport of GBA are those from GBA and/or those from SCARB2.

In some embodiments, the GBA-encoding constructs include polynucleotides that encode wild-type GBA with the natural signal peptide (a 39-amino acid GBA signal peptide as described in Sorge et al., Am. J. Hum. Genet. 41:1016-1024 (1987)). In some embodiments, the GBA-encoding constructs include polynucleotides that encode wild-type GBA without the natural signal peptide. In some embodiments, the transgene encoding secreted GBA comprises a modified signal peptide. In some embodiments, the GBA comprising a modified signal peptide has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of SEQ ID NO: 5. In some embodiments, the transgene encoding secreted GBA comprising a modified signal peptide has a nucleic acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO: 11. In some embodiments, the modified signal peptide has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of MGIPMGKSMLVLLTFLAFASCCIA (SEQ ID NO: 12).

In some embodiments, the modified signal peptide is encoded by a polynucleotide having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO: 13, as shown below.

(SEQ ID NO: 13)
ATGGGGATTCCTATGGGCAAGTCTATGCTGGTCCTGCTGACATTTCTGGC
CTTCGCTTCATGCTGTATCGCT

In some embodiments, the disclosed vectors include polynucleotides that encode SCARB2 with the natural signal peptide (a 26-amino acid SCARB2 signal peptide). In some embodiments, the vectors include polynucleotides that encode SCAB2 without the natural signal peptide. In some embodiments, the transgene includes a SCAB2 signal peptide. In some embodiments, the SCARB2 signal peptide has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of MGRCCFYTAGTLSLLLLVTSVTLLVA (SEQ ID NO: 49).

### Secretory Signal Peptides

Polynucleotides encoding GBA and/or SCARB2 may include one or more polynucleotides encoding a secretory signal peptide. Secretory signal peptides may have amino acid sequences of 5-30 residues in length, and may be located upstream of (i.e., 5′ to) a polynucleotide encoding GBA and/or SCARB2. These secretory signal peptides allow for the recognition of the nascent polypeptides during synthesis by signal recognition particles resulting in translocation to the ER, packaging into transport vesicles, and finally, secretion. Exemplary secretory signal peptides for protein secretion are those from GBA, SCARB2, IGF-II, alpha-1 antitrypsin, IL-2, IL-6, CD5, immunoglobulins, trypsinogen, serum albumin, prolactin, elastin, tissue plasminogen activator signal peptide (tPA-SP), and insulin. In some embodiments, pluripotent cells (e.g., CD34+ cells) expressing a secreted form of GBA and/or SCARB2 may be utilized as a therapeutic strategy to correct an enzyme deficiency (e.g., GBA) by infusing the missing enzyme into the bloodstream. As the blood perfuses patient tissues, GBA and/or SCARB2 are taken up by cells and transported to the lysosome, where the GBA acts to eliminate glucocerebroside that has accumulated in the lysosomes due to the GBA deficiency. For lysosomal enzyme replacement therapy to be effective, the therapeutic enzyme (e.g., GBA) must be delivered to lysosomes in the appropriate cells in tissues where the storage defect is manifest. Conventional lysosomal enzyme replacement therapeutics are delivered using carbohydrates naturally attached to the protein to engage specific receptors on the surface of the target cells. One receptor, the cation-independent mannose-6-phosphate (M6P) receptor (CI-MPR), is particularly useful for targeting replacement lysosomal enzymes as the CI-MPR is present on the surface of most cell types.

### Glycosylation Independent Lysosomal Targeting

Glycosylation Independent Lysosomal Targeting (GILT) technology can be utilized to target therapeutic enzymes (e.g., GBA and/or SCARB2) to lysosomes. Specifically, the GILT technology uses a peptide tag instead of M6P to engage the CI-MPR for lysosomal targeting. Typically, a GILT tag is a protein, peptide, or other moiety that binds the CI-MPR in a mannose-6-phosphate independent manner.

Advantageously, this technology mimics the normal biological mechanism for uptake of lysosomal enzymes, yet does so in a manner independent of mannose-6-phosphate. In some embodiments, the GBA is secreted as a GBA fusion protein containing GBA and a GILT tag. In some embodiments, the SCARB2 is secreted as a SCARB2 fusion protein containing SCARB2 and a GILT tag. In some embodiments, a GILT tag is derived from human insulin-like growth factor II (IGFII). Human IGF-II is a high affinity ligand for the CI-MPR; also referred to as IGF-II receptor. Binding of GILT-tagged therapeutic enzymes to the M6P/IGF-II receptor targets the protein to the lysosome via the endocytic pathway. A detailed description of GILT technology and the GILT tag can be found in U.S. Publication Nos. 20030082176, 20040006008, 20040005309, 20050281805, and 2009043207 the teachings of all of which are hereby incorporated by references in their entireties.

### Furin-Resistant GILT Tag

The IGF-II derived GILT tag may be subjected to proteolytic cleavage by furin during production in mammalian cells. Furin protease typically recognizes and cleaves a cleavage site having a consensus sequence Arg-X-X-Arg, where X is any amino acid. The cleavage site is positioned after the carboxy-terminal arginine (Arg) residue in the sequence. In some embodiments, a furin cleavage site has a consensus sequence Lys/Arg-X-X-X-Lys/Arg-Arg, where X is any amino acid. The cleavage site is positioned after the carboxy terminal arginine (Arg) residue in the sequence. The mature human IGF-II peptide sequence is shown below.

(SEQ ID NO: 22)
AYRPSETLCGGELVDTLQFVCGDRGFYFSRPASRVSRRSRGIVEECCFRS
CDLALLETYCATPAKSE

The mature human IGF-II contains two potential overlapping furin cleavage sites between residues 34-40 (bolded). Modified GILT tags that are resistant to cleavage by furin still retain ability to bind to the CI-MPR in a mannose-6-phosphate-independent manner. Specifically, furin-resistant GILT tags can be designed by mutating the amino acid sequence at one or more furin cleavage sites such that the mutation abolishes at least one furin cleavage site. Thus, in some embodiments, a furin-resistant GILT tag is a furin-resistant IGF-II mutein containing a mutation that abolishes at least one furin protease cleavage site or changes a sequence adjacent to the furin protease cleavage site such that the furin cleavage is prevented, inhibited, reduced or slowed down as compared to a wild-type IGF-II peptide (e.g., wild-type human mature IGF-II). A suitable mutation does not impact the ability of the furin-resistant GILT tag to bind to the human cation-independent mannose-6-phosphate receptor. In some embodiments, a furin-resistant IGF-II mutein suitable for use in conjunction with the compositions and methods described herein binds to the human cation-independent mannose-6-phosphate receptor in a mannose-6-phosphate-independent manner with a dissociation constant of 10⁻⁷ M or less (e.g., 10⁻⁸, 10⁻⁹, 10⁻¹⁰, 10⁻¹¹, or less) at pH 7.4. In some embodiments, a furin-resistant IGF-II mutein contains a mutation within a region corresponding to amino acids 30-40 (e.g., 31-40, 32-40, 33-40, 34-40, 30-39, 31-39, 32-39, 34-37, 32-39, 33-39, 34-39, 35-39, 36-39, 37-40, 34-40) of SEQ ID NO: 22. In some embodiments, a suitable mutation abolishes at least one furin protease cleavage site. A mutation can be amino acid substitutions, deletions, or insertions. For example, any one amino acid within the region corresponding to residues 30-40 (e.g., 31-40, 32-40, 33-40, 34-40, 30-39, 31-39, 32-39, 34-37, 32-39, 33-39, 34-39, 35-39, 36-39, 37-40, 34-40) of SEQ ID NO: 22 can be substituted with any other amino acid or deleted. For example, substitutions at position 34 may affect furin recognition of the first cleavage site. Insertion of one or more additional amino acids within each recognition site may abolish one or both furin cleavage sites. Deletion of one or more of the residues in the degenerate positions may also abolish both furin cleavage sites.

In some embodiments, a furin-resistant IGF-II mutein contains amino acid substitutions at positions corresponding to Arg37 or Arg40 of SEQ ID NO: 22. In some embodiments, a furin-resistant IGF-II mutein contains a Lys or Ala substitution at positions Arg37 or Arg40. Other substitutions are possible, including combinations of Lys and/or Ala mutations at both positions 37 and 40, or substitutions of amino acids other than Lys or Ala. In some embodiments, the furin-resistant IGF-II mutein suitable for use in conjunction with the compositions and methods described herein may contain additional mutations. For example, up to 30% or more of the residues of SEQ ID NO: 22 may be changed (e.g., up to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or more residues may be changed). Thus, a furin-resistant IGF-II mutein suitable for use in conjunction with the compositions and methods described herein may have an amino acid sequence at least 70%, including at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, identical to SEQ ID NO: 22. In some embodiments, a furin-resistant IGF-II mutein suitable for use in conjunction with the compositions and methods described herein is targeted specifically to the CI-MPR. Particularly useful are mutations in the IGF-II polypeptide that result in a protein that binds the CI-MPR with high affinity (e.g., with a dissociation constant of 10⁻⁷ M or less at pH 7.4) while binding other receptors known to be bound by IGF-II with reduced affinity relative to native IGF-II. For example, a furin-resistant IGF-II mutein suitable for use in conjunction with the compositions and methods described herein can be modified to have diminished binding affinity for the IGF-I receptor relative to the affinity of naturally-occurring human IGF-II for the IGF-I receptor. Additional mutational strategies have been utilized and are discussed at length in the U.S. Publication No. 2009043207, which is hereby incorporated by reference. For example, substitution of IGF-II residues Tyr 27 with Leu, Leu 43 with Val, or Ser 26 with Phe diminishes the affinity of IGF-II for the IGF-I receptor by 94-, 56-, and 4-fold respectively (Torres et al., J. Mol. Biol. 248:385-401 (1995)). Deletion of residues 1-7 of human IGF-II resulted in a 30-fold decrease in affinity for the human IGF-I receptor and a concomitant 12-fold increase in affinity for the rat IGF-II receptor (Hashimoto et al., J. Biol. Chem. 270:18013-8 (1995)). The NMR structure of IGF-II shows that Thr 7 is located near residues 48 Phe and 50 Ser, as well as near the 9 Cys-4 7 Cys disulfide bridge. It is thought that interaction of Thr 7 with these residues can stabilize the flexible N-terminal hexapeptide required for IGF-I receptor binding (Terasawa et al., EMBO J. 13:5590-7 (1994)). At the same time, this interaction can modulate binding to the IGF-II receptor. Truncation of the C-terminus of IGF-II (residues 62-67) also appears to lower the affinity of IGF-II for the IGF-I receptor by 5-old (Roth et al., Biochem. Biophys. Res. Commun. 181:907-14 (1991)). The binding surfaces for the IGF-I and cation-independent M6P receptors are on separate faces of IGF-II. Based on structural and mutational data, functional cation-independent M6P binding domains can be constructed that are substantially smaller than human IGF-II. For example, the amino terminal amino acids (e.g., 1-7 or 2-7) and/or the carboxy terminal residues 62-67 can be deleted or replaced. Additionally, amino acids 29-40 can likely be eliminated or replaced without altering the folding of the remainder of the polypeptide or binding to the cation-independent M6P receptor. Thus, a targeting moiety including amino acids 8-28 and 41-61 can be constructed. These stretches of amino acids could perhaps be joined directly or separated by a linker. Alternatively, amino acids 8-28 and 41-61 can be provided on separate polypeptide chains. Comparable domains of insulin, which are homologous to IGF-II and have a tertiary structure closely related to the structure of IGF-II, have sufficient structural information to permit proper refolding into the appropriate tertiary structure, even when present in separate polypeptide chains (Wang et al., Trends Biochem. Sci. 16:279-281 (1991)). Thus, for example, amino acids 8-28, or a conservative substitution variant thereof, could be fused to a lysosomal enzyme; the resulting fusion protein could be admixed with amino acids 41-61, or a conservative substitution variant thereof, and administered to a patient. IGF-II can also be modified to minimize binding to serum IGF-binding proteins (Baxter, Am. J. Physiol Endocrinol Metab. 278:967-76(2000)) to avoid sequestration of IGF-II/GILT constructs. A number of studies have localized residues in IGF-II necessary for binding to IGF-binding proteins. Constructs with mutations at these residues can be screened for retention of high affinity binding to the M6P/IGF-II receptor and for reduced affinity for IGF binding proteins. For example, replacing Phe 26 of IGF-II with Ser is reported to reduce affinity of IGF-II for IGFBP-1 and -6, with no effect on binding to the M6P/IGF-II receptor (Bach et al., J. Biol. Chem. 268:9246-54 (1993)). Other substitutions, such as Lys for Glu 9, can also be advantageous. The analogous mutations, separately or in combination, in a region of IGF-I that is highly conserved with IGF-II result in large decreases in IGF-BP binding (Magee et al., Biochemistry 38:15863-70 (1999)).

An alternate approach is to identify minimal regions of IGF-II that can bind with high affinity to the M6P/IGF-II receptor. The residues that have been implicated in IGF-II binding to the M6P/IGF-II receptor mostly cluster on one face of IGF-II (Terasawa et al., EMBO J. 13:5590-7 (1994)). Although IGF-II tertiary structure is normally maintained by three intramolecular disulfide bonds, a peptide incorporating the amino acid sequence on the M6P/IGF-II receptor binding surface of IGF-II can be designed to fold properly and have binding activity. Such a minimal binding peptide is a highly preferred lysosomal targeting domain. For example, a preferred lysosomal targeting domain is amino acids 8-67 of human IGF-II. Designed peptides, based on the region around amino acids 48-55, which bind to the M6P/IGF-II receptor, are also desirable lysosomal targeting domains. Alternatively, a random library of peptides can be screened for the ability to bind the M6P/IGF-II receptor either via a yeast two hybrid assay, or via a phage display type assay.

Many furin-resistant IGF-II muteins described herein have reduced or diminished binding affinity for the insulin receptor. Thus, in some embodiments, a peptide tag suitable for use in conjunction with the compositions and methods described herein has reduced or diminished binding affinity for the insulin receptor relative to the affinity of naturally occurring human IGF-II for the insulin receptor. In some embodiments, peptide tags with reduced or diminished binding affinity for the insulin receptor suitable for use in conjunction with the compositions and methods described herein include peptide tags having a binding affinity for the insulin receptor that is more than 1.5-fold, 2-fold, 3-fold, 4-fold, 5-fold, 6-fold, 7-fold, 8-fold, 9-fold, 10-fold, 12-fold, 14-fold, 16-fold, 18-fold, 20-fold, 50-fold, 100-fold less than that of the wild-type mature human IGF-II. The binding affinity for the insulin receptor can be measured using various in vitro and in vivo assays known in the art.

In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 23, as shown below.

(SEQ ID NO: 23)
GGGGAGGGGAGGGGAGGGGAGGGPSLCGGELVDTLQFVCGDRGFYFSRPA
SRVSARSRGIVEECCFRSCDLALLETYCATPAKSE

In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 24, as shown below.

(SEQ ID NO: 24)
GAPGGGSPAPAPTPAPAPTPAPAGGGPSGAPLCGGELVDTLQFVCGDRGF
YFSRPASRVSARSRGIVEECCFRSCDLALLETYCATPAKSE

In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 25, as shown below.

(SEQ ID NO: 25)
GAPGGSPAGSPTSTEEGTSESATPESGPGTSTEPSEGSAPGSPAGSPTST
GPSGAPLCGGELVDTLQFVCGDRGFYFSRPASRVSARSRGIVEECCFRSC
DLALLETYCATPAKSE

In some embodiments, the GILT tag is encoded by a nucleic acid sequence having at least 85% sequence identity (e.g., 85%, 90%, 95%, 96%, 97%, 98%, 99%, or greater, sequence identity) to the nucleic acid sequence of SEQ ID NO: 26, as shown below.

(SEQ ID NO: 26)
GGCGGAGGCGGAGCTGGTGGGGGGGGAGCAGGCGGTGGTGGTGCAGGGGG
GGGAGGTGCTGGCGGAGGACCATCTCTTTGTGGCGGAGAACTGGTGGACA
CCCTGCAGTTCGTGTGTGGCGACAGAGGCTTCTACTTTAGCAGACCCGCC
AGCAGAGTGTCCGCCAGATCTAGAGGAATCGTGGAAGAGTGCTGCTTCAG
AAGCTGCGACCTGGCACTGCTGGAAACCTACTGTGCCACACCAGCCAAGA
GCGAGTGATG

In some embodiments, the GILT tag is encoded by a nucleic acid sequence having at least 85% sequence identity (e.g., 85%, 90%, 95%, 96%, 97%, 98%, 99%, or greater, sequence identity) to the nucleic acid sequence of SEQ ID NO: 27, as shown below.

(SEQ ID NO: 27)
GGAGCACCAGGCGGAGGATCTCCAGCTCCTGCTCCTACACCAGCTCCAGC
ACCGACGCCTGCTCCAGCTGGCGGAGGACCTTCTGGTGCACCTCTTTGTG
GCGGAGAGCTGGTGGATACCCTGCAGTTCGTGTGTGGCGACCGGGGCTTC
TACTTTAGCAGACCTGCCAGCAGAGTGTCCGCCAGATCTAGAGGCATCGT
GGAAGAGTGCTGCTTCAGAAGCTGCGACCTGGCACTGCTGGAAACCTACT
GTGCCACACCAGCCAAGAGCGAGTGATGA

In some embodiments, the GILT tag is encoded by a nucleic acid sequence having at least 85% sequence identity (e.g., 85%, 90%, 95%, 96%, 97%, 98%, 99%, or greater, sequence identity) to the nucleic acid sequence of SEQ ID NO: 28, as shown below.

(SEQ ID NO: 28)
GGAGCACCAGGCGGATCTCCAGCAGGATCTCCAACCTCTACCGAGGAAGG
CACAAGCGAGTCTGCCACACCTGAGTCTGGACCTGGCACAAGCACAGAGC
CTAGCGAAGGATCTGCCCCAGGTTCTCCTGCCGGCTCTCCTACAAGTACA
GGACCTTCTGGCGCTCCACTGTGTGGCGGAGAACTGGTGGATACCCTGCA
GTTCGTGTGCGGCGACAGAGGCTTCTACTTTAGCAGACCCGCCAGCAGAG
TGTCCGCCAGATCTAGAGGAATCGTGGAAGAGTGCTGCTTCAGAAGCTGC
GATCTGGCACTGCTGGAAACCTACTGTGCCACACCAGCCAAGAGCGAGTG
ATGA

### ApoE Tag for Blood-Brain Barrier Penetrance of Secreted GBA

In some embodiments, the GBA (e.g., secreted GBA fusion protein) and/or the SCARB2 protein is modified to penetrate the blood-brain barrier (BBB). Modifications for mediating BBB penetrance are well known in the art. Exemplary modifications are the use of tags containing the receptor binding (Rb) domain (amino acid residues 148-173 of SEQ ID NO: 29) of apolipoprotein E (ApoE). The complete ApoE peptide sequence is shown below.

(SEQ ID NO: 29)
MKVLWAALLVTFLAGCQAKVEQAVETEPEPELRQQTEWQSGQRWELALGR
FWDYLRWVQTLSEQVQEELLSSQVTQELRALMDETMKELKAYKSELEEQL
TPVAEETRARLSKELQAAQARLGADMEDVCGRLVQYRGEVQAMLGQSTEE
LRVRLASHLRKLRKRLLRDADDLQKRLAVYQAGAREGAERGLSAIRERLG
PLVEQGRVRAATVGSLAGQPLQERAQAWGERLRARMEEMGSRTRDRLDEV
KEQVAEVRAKLEEQAQQIRLQAEAFQARLKSWFEPLVEDMQRQWAGLVEK
VQAAVGTSAAPVPSDNH

ApoE is an important protein involved in lipid transport, and its cellular internalization is mediated by several members of the low-density lipoprotein (LDL) receptor gene family, including the LDL receptor, very low-density lipoprotein receptor (VLDLR), and LDL receptor-related proteins (LRPs, including LRP1, LRP2, and LRP8). The LDL receptor is found to be highly expressed in brain capillary endothelial cells (BCECs), with down-regulated expression observed in peripheral vessels. Restricted expressions of LRPs and VLDLR have also been shown prominently in the liver and brain when they have been detected in BCECs, neurons, and glial cells. Several members of the low-density lipoprotein receptor family (LDLRf) proteins, including LRP1 and VLDLR, but not LDLR, are highly expressed in BBB-forming BCECs. These proteins can bind ApoE to facilitate their transcytosis into the abluminal side of the BBB.

In addition, receptor-associated protein (RAP), an antagonist as well as a ligand for both LRP1 and VLDLR, has been shown to have higher permeability across the BBB than transferrin in vivo and in vitro (Pan et al., J. Cell Sci. 117:5071-8 (2004)), indicating that these lipoprotein receptors (LDLRf) can represent efficient BBB delivery targets despite their lower expression than the transferrin receptor. As described herein, a potent receptor-binding peptide (Rb) derived from ApoE, has the ability to translocate protein across the BBB into the brain when engineered as fusion proteins. This method can therefore function to selectively open the BBB for therapeutic agents (e.g., soluble GBA) when engineered as a fusion protein. This peptide can be readily attached to diagnostic or therapeutic agents without jeopardizing their biological functions or interfering with the important biological functions of ApoE due to the utilization of the Rb domain of ApoE, rather than the entire ApoE protein. This pathway is also an alternative uptake pathway that can facilitate further/secondary distribution within the brain after the agents reach the CNS due to the widespread expression of LDLRf members in brain parenchyma. Regardless of application strategies, e.g., enzyme replacement therapy or cell-based, gene-based therapy, both the quantity and distribution of therapeutics within the brain parenchyma will have a significant impact on the clinical outcome of disease treatment. The development of and a detailed description of the use of the Rb domain of ApoE in targeted delivery of proteins across the BBB can be found in U.S. Publication No. 20140219974, which is hereby incorporated by reference in its entirety.

In some embodiments, the secreted GBA fusion protein and/or the SCARB2 protein has a peptide sequence containing the LDLRf receptor-binding domain (Rb) of SEQ ID NO: 29, or a fragment, variant, or oligomer thereof. An exemplary receptor-binding domain can be found in the N-terminus of ApoE, for example, between amino acid residues 1 to 191 of SEQ ID NO: 29, between amino acid residues 25 to 185 of SEQ ID NO: 29, between amino acid residues 50 to 180 of SEQ ID NO: 29, between amino acid residues 75 to 175 of SEQ ID NO: 29, between amino acid residues 100 to 170 of SEQ ID NO: 29, or between amino acid residues 125 to 165 of SEQ ID NO: 29. An exemplary receptor binding domain has the amino acid sequence of residues 159 to 167 of SEQ ID NO: 29.

In some embodiments, the peptide sequence containing the receptor-binding domain of ApoE can include at least one amino acid mutation, deletion, addition, or substitution. In some embodiments, the amino acid substitutions can be a combination of two or more mutations, deletions, additions, or substitutions. In some embodiments, the at least one substation is a conservative substitution. In some embodiments, the at least one amino acid addition includes addition of a selected sequence already found in the Rb domain of ApoE. A person of ordinary skill in the art will recognize suitable modifications that can be made to the sequence while retaining some degree of the biochemical activity for transport across the BBB.

### Vectors for the Expression of GBA and SCARB2

In addition to achieving high rates of transcription and translation, stable expression of an exogenous gene in a mammalian cell can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are disclosed in, e.g., WO 1994/011026 and are incorporated herein by reference. Expression vectors for use in the compositions and methods described herein contain a polynucleotide sequence that encodes GBA and/or SCARB2, as well as, e.g., additional sequence elements used for the expression of these agents and/or the integration of these polynucleotide sequences into the genome of a mammalian cell. Certain vectors that can be used for the expression of GBA and/or SCARB2 include plasmids that contain regulatory sequences, such as promoter and enhancer regions, which direct gene transcription. Other useful vectors for expression of GBA and/or SCARB2 contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin.

Expression vectors for use in the compositions and methods described herein may express GBA and/or SCARB2 from monocistronic or polycistronic expression cassettes. A monocistronic expression cassette contains a polynucleotide sequence that encodes a single gene. Pluripotent cells (e.g., CD34+ cells) described herein can be transfected with multiple plasmids, for example, each containing a monocistronic expression cassette, or with a single plasmid containing more than one monocistronic expression cassette. Polycistronic expression cassettes can be used to simultaneously express two or more proteins from a single transcript (e.g., GBA and SCARB2 proteins). Polycistronic expression cassettes include bicistronic expression cassettes, which can be used to generate two proteins from a single transcript and may include IRES sequences to recruit ribosomes to initiate translation from a region of the mRNA other than the 5′ cap.

Foot-and-mouth disease virus 2A (FMDV 2A or F2A) polynucleotides can be utilized to express two or more genes (e.g., 2 genes, 3 genes, 4 genes, 5 genes or more), and can be used in bicistronic or polycistronic expression cassettes to produce equimolar levels of multiple genes from the same transcript. FMDV 2A mediates a cotranslational cleavage event, which separates proteins linked by 2A sequences, and multiple 2A sequences may be used in one vector. For an example of the use of FMDV 2A to express multiple proteins, see Ryan et al., EMBO Journal 13:928 (1994), the disclosure of which is incorporated herein by reference as it pertains to the use of FMDV 2A sequences. 2A-like sequences from other viruses can also be used in the compositions and methods described herein, including the 2A-like sequences from equine rhinitis A virus (E2A), porcine teschovirus-1 (P2A), and Thosea asigna virus (T2A), as described in Szymczak and Vignali, Expert Opin Biol Ther. 5:627 (2005), Szymczak et al., Nat Biotechnol. 22:589 (2004), and Osborn et al., Molecular Therapy 12:569 (2005), the disclosures of which are incorporated herein by reference as they pertain to the use of 2A-like sequences in gene expression.

### Cell-Penetrating Peptides

Cell-penetrating peptides (CPPs) are polypeptides that contain an abundance of cationic amino acids and, as such, engage in strong ionic contacts with the negatively charged exterior of the mammalian cell membrane. These compounds are capable of penetrating the cell membrane by one of a variety of mechanisms, including destabilization of the membrane structure, pore formation, endocytosis, and macropinocytosis, among others. CPPs have been shown not only to translocate across the mammalian cell membrane, but are also capable of delivering other molecules to which these compounds are covalently bound into the mammalian cell interior. The use of CPPs is described in Snyder, et al., Pharm. Res. 21:389-393 (2004), the teachings of which are incorporated by reference. Examples of cell penetrating peptides that can be fused to a GBA and/or SCARB2 protein are provided in SEQ ID NOs: 30-48. For instance, one can conjugate the N-terminal amine of GBA or SCARB2 to the C-terminal carboxylate of a cell penetrating peptide by formation of an amide bond using amide-bond forming reagents and processes known in the art. In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of any one of SEQ ID NOs: 30-48. In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of RQIKWFQNRRMKWKK (SEQ ID NO: 30). In some embodiments, the CPP has the amino acid sequence of SEQ ID NO: 30.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of YGRKKRRQRRR (SEQ ID NO: 31). In some embodiments, the CPP has the amino acid sequence of SEQ ID NO: 31).

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of RGGRLSYSRRRFSTSTGR (SEQ ID NO: 32). In some embodiments, the CPP has the amino acid sequence of SEQ ID NO: 32.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of RRLSYSRRRF (SEQ ID NO: 33). In some embodiments, the CPP has the amino acid sequence of SEQ ID NO: 33.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of PIRRRKKLRRLK (SEQ ID NO: 34). In some embodiments, the CPP has the amino acid sequence of SEQ ID NO: 34.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of RRQRRTSKLMKR (SEQ ID NO: 35). In some embodiments, the CPP has the amino acid sequence of SEQ ID NO: 35.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of RRRRNRTRRNRRRVR (SEQ ID NO: 36). In some embodiments, the CPP has the amino acid sequence of SEQ ID NO: 36.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of KMTRAQRRAAARRNRWTAR (SEQ ID NO: 37). In some embodiments, the CPP has the amino acid sequence of SEQ ID NO: 37.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of TRRQRTRRARRNR (SEQ ID NO: 38). In some embodiments, the CPP has the amino acid sequence of SEQ ID NO: 38.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of GRKKRRQRRRPPQ (SEQ ID NO: 39). In some embodiments, the CPP has the amino acid sequence of SEQ ID NO: 39.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of GRRRRRRRRRPPQ (SEQ ID NO: 40). In some embodiments, the CPP has the amino acid sequence of SEQ ID NO: 40.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of GWTLNSAGYLLGKINLKALAALAKKIL (SEQ ID NO: 41). In some embodiments, the CPP has the amino acid sequence of SEQ ID NO: 41.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of KLALKLALKLALALKLA (SEQ ID NO: 42). In some embodiments, the CPP has the amino acid sequence of SEQ ID NO: 42.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of MGLGLHLLVLAAALQGAWSQPKKKRKV (SEQ ID NO: 43). In some embodiments, the CPP has the amino acid sequence of SEQ ID NO: 43.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of GALFLGWLGAAGSTMGAWSQPKKKRKV (SEQ ID NO: 44). In some embodiments, the CPP has the amino acid sequence of SEQ ID NO: 44.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of GALFLGFLGAAGSTMGAWSQPKKKRKV (SEQ ID NO: 45). In some embodiments, the CPP has the amino acid sequence of SEQ ID NO: 45.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of GALFLGFLGAAGSTMGAWSQPKSKRKV (SEQ ID NO: 46). In some embodiments, the CPP has the amino acid sequence of SEQ ID NO: 46.

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of KETWWETWWTEWSQPKKKRKV (SEQ ID NO: 47).

In some embodiments, the CPP has an amino acid sequence having at least 85% (e.g., at least 86%, 87%, 88%, 89%, 90%, 91%, 92%, 93%, 94%, 95%, 96%, 97%, 98%, 99%, or more) sequence identity to the amino acid sequence of KETWFETWFTEWSQPKKKRKV (SEQ ID NO: 48). In some embodiments, the CPP has the amino acid sequence of SEQ ID NO: 48.

### Viral Vectors for Expression of GBA and SCARB2

Viral genomes provide a rich source of vectors that can be used for the efficient delivery of exogenous genes into a mammalian cell. Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### AAV Vectors

Nucleic acids of the compositions and methods described herein may be incorporated into rAAV vectors and/or virions in order to facilitate their introduction into a cell. AAV vectors can be used in the central nervous system, and appropriate promoters and serotypes are discussed in Pignataro et al., J Neural Transm. 125:575-89 (2017), the disclosure of which is incorporated herein by reference as it pertains to promoters and AAV serotypes useful in CNS gene therapy. rAAV vectors useful in the compositions and methods described herein are recombinant nucleic acid constructs that include (1) a heterologous sequence to be expressed (e.g., a polynucleotide encoding GBA and/or SCARB2) and (2) viral sequences that facilitate integration and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part, but retain functional flanking ITR sequences. The AAV ITRs may be of any serotype suitable for a particular application. Methods for using rAAV vectors are described, for example, in Tal et al., J. Biomed. Sci. 7:279 (2000), and Monahan and Samulski Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2, and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10 and rh74. For targeting cells located in or delivered to the central nervous system, AAV2, AAV9, and AAV10 may be particularly useful. Construction and use of AAV vectors and AAV proteins of different serotypes are described, for example, in Chao et al., Mol. Ther. 2:619 (2000); Davidson et al., PNAS 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype pseudotyped with a capsid gene derived from a serotype other than the given serotype (e.g., AAV1, AAV2, AAV3, AAV4, AAV5, AAV6, AAV7, AAV8, AAV9, and AAV10, among others). Techniques involving the construction and use of pseudotyped rAAV virions are known in the art and are described, for example, in Duan et al., J. Virol. 75:7662 (2001); Halbert et al., J. Virol. 74:1524 (2000); Zolotukhin et al., Methods 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet. 25:436 (2000) and Kolman and Stemmer, Nat. Biotechnol. 19:423 (2001).

### Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene. An overview of optimization strategies for lentiviral vectors is provided in Delenda, The Journal of Gene Medicine 6:S125 (2004), the disclosure of which is incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans co-expression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

A lentiviral vector used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen lentiviral vector described in Lu et al., Journal of Gene Medicine 6:963 (2004) may be used to express the DNA molecules and/or transduce cells.

A lentiviral vector used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). It will be readily apparent to one skilled in the art that optionally one or more of these regions is substituted with another region performing a similar function.

GBA and SCARB2 are required to be expressed at sufficiently high levels. Transgene expression is driven by a promoter sequence. Optionally, the lentiviral vector includes a CMV promoter. The promoter may also be EF1α or PGK promoter. In another embodiment, the promoter is a microglia-specific promoter, e.g., CD68 promoter, CX3CR1 promoter, ITGAM promoter, AIF1 promoter, P2Y12 promoter, TMEM119 promoter, or CSF1R promoter. In some embodiments, the promoter is an MND promoter (e.g., MND promoter disclosed herein). A person skilled in the art will be familiar with a number of promoters that will be suitable in the vector constructs described herein.

Enhancer elements can be used to increase expression of modified DNA molecules or increase the lentiviral integration efficiency. The lentiviral vector used in the methods and compositions described herein may include a nef sequence. The lentiviral vector used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The lentiviral vector used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to lentiviral vector results in a substantial improvement in the level of transgene expression from several different promoters, both in vitro and in vivo. The lentiviral vector used in the methods and compositions described herein may include both a cPPT sequence and WPRE sequence. The vector may also include an internal ribosome entry site (IRES) sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther. 8:811 (2001), Osborn et al., Molecular Therapy 12:569 (2005), Szymczak et al., Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

### Viral Regulatory Elements

The viral regulatory elements are components of delivery vehicles used to introduce nucleic acid molecules into a host cell. The viral regulatory elements are optionally retroviral regulatory elements. For example, the viral regulatory elements may be the LTR and gag sequences from HSC1 or MSCV. The retroviral regulatory elements may be from lentiviruses or they may be heterologous sequences identified from other genomic regions.

One skilled in the art would also appreciate that as other viral regulatory elements are identified, these may be used with the nucleic acid molecules described herein.

### Methods for the Delivery of Exogenous Nucleic Acids to Target Cells

Techniques that can be used to introduce a polynucleotide, such as codon-optimized DNA or RNA (e.g., mRNA, tRNA, siRNA, miRNA, shRNA, chemically modified RNA) into a mammalian cell are well known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells are the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for example, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US 2010/0227406, the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107:1870 (2010), the disclosure of which is incorporated herein by reference.

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Modulation of Gene Expression Using Gene Editing Techniques

### Disruption of Endogenous GBA

In some embodiments, endogenous GBA is disrupted (e.g., in a subject undergoing treatment, such as in a population of neurons in a subject undergoing treatment, or in the pluripotent cells to be administered to the subject). Exemplary methods for disrupting endogenous GBA expression are those in which an inhibitory RNA molecule is administered to the subject, or contacted with a population of neurons in the subject or the population of pluripotent cells to be administered to the subject. The inhibitory RNA molecule may function to disrupt endogenous GBA expression, for example, act by way of the RNA interference (RNAi) pathway. An inhibitory RNA molecule can decrease the expression level (e.g., protein level or mRNA level) of endogenous GBA. For example, an inhibitory RNA molecule includes a short interfering RNA, short hairpin RNA, and/or a microRNA that targets full-length endogenous GBA. A siRNA is a double-stranded RNA molecule that typically has a length of about 19-25 base pairs. A shRNA is a RNA molecule including a hairpin turn that decreases expression of target genes via RNAi. shRNAs can be delivered to cells in the form of plasmids, e.g., viral or bacterial vectors, e.g., by transfection, electroporation, or transduction). A microRNA is a non-coding RNA molecule that typically has a length of about 22 nucleotides. miRNAs bind to target sites on mRNA molecules and silence the mRNA, e.g., by causing cleavage of the mRNA, destabilization of the mRNA, or inhibition of translation of the mRNA. An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability, or decrease immunogenicity.

In some embodiments, the inhibitory RNA molecule decreases the level and/or activity or function of endogenous GBA. In embodiments, the inhibitory RNA molecule inhibits expression of endogenous GBA. In other embodiments, the inhibitor RNA molecule increases degradation of endogenous GBA and/or decreases the stability of endogenous GBA. The inhibitory RNA molecule can be chemically synthesized or transcribed in vitro.

In some embodiments, the endogenous GBA is disrupted in the pluripotent stem cells containing the GBA transgene using, for example, the gene editing techniques described herein. In some embodiments, the endogenous GBA is globally disrupted in the subject using, for example, the gene editing techniques described herein. In some embodiments, the endogenous GBA is disrupted in a population of neurons in the subject using, for example, the gene editing techniques described herein. In some embodiments, disruption of endogenous GBA in the subject, neurons, and/or pluripotent cells containing the GBA transgene occurs prior to administration of the pluripotent stem cells to the subject.

The making and use of inhibitory therapeutic agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press 2010.

### Nuclease-Mediated Gene Transfer

Another useful tool for the disruption and integration of target genes into the genome of a pluripotent stem cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific Cas9-mediated DNA cleavage can be engendered in a foreign polynucleotide because the interaction that brings Cas9 within close proximity of the target DNA molecule is governed by RNA: DNA hybridization. As a result, one can theoretically design a CRISPR/Cas system to cleave any target DNA molecule of interest (e.g., endogenous GBA). This technique has been exploited in order to edit eukaryotic genomes (Hwang et al. Nature Biotechnology 31:227 (2013), the disclosure of which is incorporated herein by reference) and can be used as an efficient means of site-specifically editing pluripotent stem cell genomes in order to cleave DNA prior to the incorporation of a gene encoding a target gene. The use of CRISPR/Cas to modulate gene expression has been described in, e.g., U.S. Pat. No. 8,697,359, the disclosure of which is incorporated herein by reference. Alternative methods for disruption of a target DNS by site-specifically cleaving genomic DNA prior to the incorporation of a gene of interest in a pluripotent stem cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al. Nature Reviews Genetics 11:636 (2010); and in Joung et al. Nature Reviews Molecular Cell Biology 14:49 (2013), the disclosure of both of which are incorporated herein by reference. In some embodiments, the endogenous GBA may be disrupted in the pluripotent stem cells containing the GBA transgene and/or SCARB2 using these gene editing techniques described herein.

### Transposon-Mediated Gene Transfer

In addition to viral vectors, a variety of additional tools have been developed that can be used for the incorporation of exogenous genes into pluripotent stem cells (e.g., CD34+ cells). One such method that can be used for incorporating polynucleotides encoding target genes into pluripotent stem cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In certain cases, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Transposon systems include the piggybac transposon (described in detail in, e.g., WO 2010/085699) and the sleeping beauty transposon (described in detail in, e.g., US2005/0112764), the disclosures of each of which are incorporated herein by reference.

### Methods of Treatment

### Selection of Subjects

Subjects that may be treated as described herein are subjects having or at risk of developing Gaucher disease. The type of Gaucher disease may be GBA-associated Gaucher disease, sporadic Gaucher disease, or Gaucher disease caused by an environmental toxin, e.g., herbicides or pesticides. The compositions and methods described herein can be used to treat patients with normal GBA activity, reduced GBA activity, and patients whose GBA mutational status and/or GBA activity level is unknown. The compositions and methods described herein may also be administered as a preventative treatment to patients at risk of developing Gaucher disease, e.g., patients with a GBA mutation, patients with reduced GBA activity, or patients exposed to an environmental toxin associated with Gaucher disease. Patients at risk for Gaucher disease may show early symptoms of Gaucher disease or may not yet be symptomatic when treatment is administered.

### Routes of Administration

The agents, cells, and compositions described herein may be administered to a subject with Gaucher disease by a variety of routes, such as intracerebroventricularly, stereotactically, intravenously, intraosseously, or by means of a bone marrow transplant. In some embodiments, the cells and compositions described herein may be administered to a subject systemically (e.g., intravenously), directly to the central nervous system (CNS) (e.g., intracerebroventricularly or stereotactically), or directly into the bone marrow (e.g., intraosseously). In some embodiments, the cells and compositions described herein are administered to a subject intracerebroventricularly into the cerebral lateral ventricles (a description of this method can be found in Capotondo et al., Science Advances 3:e1701211 (2017), incorporated herein by reference as it pertains to intracerebroventricular injection of hematopoietic stem and progenitor cells into the cerebral lateral ventricles of murine models). In some embodiments, the cells and compositions described herein are administered to a subject by stereotactic injection into the substantia nigra (a description of this method can be found in Altarche-Xifro et al., EBiomedicine 8:83 (2016), incorporated herein by reference as it pertains to stereotactic injection of hematopoietic stem and progenitor cells into the substantia nigra of murine models). The most suitable route for administration in any given case will depend on the particular cell or composition administered, the patient, pharmaceutical formulation methods, administration methods (e.g., administration time and administration route), the patient's age, body weight, sex, severity of the diseases being treated, the patient's diet, and the patient's excretion rate. Multiple routes of administration may be used to treat a single subject, e.g., intracerebroventricular or stereotactic injection and intravenous injection, intracerebroventricular or stereotactic injection and intraosseous injection, or intracerebroventricular or stereotactic injection and bone marrow transplant. Multiple routes of administration may be used to treat a single subject at one time, or the subject may receive treatment via one route of administration first, and receive treatment via another route of administration during a second appointment, e.g., 1 week later, 2 weeks later, 1 month later, 6 months later, or 1 year later. Cells may be administered to a subject once, or cells may be administered one or more times (e.g., 2-10 times) per week, month, or year to a subject treatment for Gaucher disease.

### Conditioning

Prior to administration of cells or compositions, it may be advantageous to deplete or ablate endogenous microglia and/or hematopoietic stem and progenitor cells. Microglia and/or hematopoietic stem and progenitor cells can be ablated through the use of chemical agents (e.g., busulfan, treosulfan, PLX3397, PLX647, PLX5622, or clodronate liposomes), irradiation, or a combination thereof. The agents used for cell ablation may be BBB penetrating (e.g., busulfan) or may lack the ability to cross the BBB (e.g., treosulfan). Exemplary microglia and/or hematopoietic stem and progenitor cells ablating agents are busulfan (Capotondo et al., PNAS 109:15018 (2012), the disclosure of which is incorporated by reference as it pertains to the use of busulfan to ablate microglia), treosulfan, PLX3397, PLX647, PLX5622, or clodronate liposomes. Other agents for the depletion of endogenous microglia and/or hematopoietic stem and progenitor cells include cytotoxins covalently conjugated to antibodies or antigen binding fragments thereof capable of binding antigens expressed by hematopoietic stem cells so as to form an antibody-drug conjugate. Cytotoxins suitable for antibody drug conjugates include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as α-amanitin and derivatives thereof), agents capable of disrupting protein biosynthesis (e.g., agents that exhibit rRNA N-glycosidase activity, such as saporin and ricin A-chain), among others known in the art. Ablation may eliminate all microglia and/or hematopoietic stem and progenitor cells, or it may reduce microglia and/or hematopoietic stem and progenitor cells numbers by at least 10% (e.g., at least 20%, 30%, 40%, 50%, 60%, 70%, 80%, 90%, 95%, or more). One or more agents to ablate microglia and/or hematopoietic stem and progenitor cells may be administered at least one week (e.g., 1, 2, 3, 4, 5, or 6 weeks or more) before administration of the cells or compositions described herein. Cells administered in the methods described herein may replace the ablated microglia and/or hematopoietic stem and progenitor cells, and may repopulate the brain following intracerebroventricular, stereotactic, intravenous, or intraosseous injection, or following bone marrow transplant. Cells administered intravenously, intraosseously, or by bone marrow transplant may cross the blood brain barrier to enter the brain and differentiate into microglia. Cells administered to the brain, e.g., cells administered intracerebroventricularly or stereotactically, can differentiate into microglia in vivo or can be differentiated into microglia ex vivo.

### Stem Cell Rescue

The methods described herein may include administering to a subject a population of CD34+ cells (e.g., embryonic stem cells, induced pluripotent stem cells, hematopoietic stem cells, and myeloid progenitor cells). These cells may be CD34+ cells that have not been modified to express the transgene encoding GBA and/or SCARB2. These cells may have disrupted endogenous GBA. The CD34+ cells may be administered systemically (e.g., intravenously), or by bone marrow transplantation to reconstitute the bone marrow compartment following conditioning as described herein. For example, these CD34+ cells may migrate to a stem cell niche and increase the quantity of cells of the hematopoietic lineage at such a site by, for example, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more. Administration may occur prior to, or following administration of the composition of the described herein.

### Selection of Donor Cells

In some embodiments, the subject is the donor. In such cases, withdrawn hematopoietic stem or progenitor cells may be re-infused into the subject (following modification (e.g., incorporation of the transgene encoding GBA and/or SCARB2, and/or disruption of endogenous GBA), such that the cells may subsequently home to hematopoietic tissue and establish productive hematopoiesis, thereby populating or repopulating a line of cells that is defective or deficient in the subject (e.g., a population of microglia). In this scenario, the transplanted hematopoietic stem or progenitor cells are least likely to undergo graft rejection, as the infused cells are derived from the patient and express the same HLA class I and class II antigens as expressed by the patient.

Alternatively, the subject and the donor may be distinct. In some embodiments, the subject and the donor are related, and may, for example, be HLA-matched. As described herein, HLA-matched donor-recipient pairs have a decreased risk of graft rejection, as endogenous T cells and NK cells within the transplant recipient are less likely to recognize the incoming hematopoietic stem or progenitor cell graft as foreign, and are thus less likely to mount an immune response against the transplant. Exemplary HLA-matched donor-recipient pairs are donors and recipients that are genetically related, such as familial donor-recipient pairs (e.g., sibling donor-recipient pairs).

In some embodiments, the subject and the donor are HLA-mismatched, which occurs when at least one HLA antigen, in particular with respect to HLA-A, HLA-B and HLA-DR, is mismatched between the donor and recipient. To reduce the likelihood of graft rejection, for example, one haplotype may be matched between the donor and recipient, and the other may be mismatched.

### Pharmaceutical Compositions and Dosing

The number of cells administered to a subject for the treatment of Gaucher disease (e.g., GBA-associated Gaucher disease) as described herein may depend, for example, on the expression level of GBA and/or SCARB2 in the cells, the patient, pharmaceutical formulation methods, administration methods (e.g., administration time and administration route), the patient's age, body weight, sex, severity of the disease being treated, and whether or not the patient has been treated with agents to ablate endogenous microglia. The number of cells administered may be, for example, 1×10⁶ cells/kg to 1×10¹² cells/kg, or more (e.g., 1×10⁷ cells/kg, 1×108 cells/kg, 1×10⁹ cells/kg, 1×10¹⁰ cells/kg, 1×10¹¹ cells/kg, 1×10¹² cells/kg, or more). Cells may be administered in an undifferentiated state, or after partial or complete differentiation into microglia. The number of CD34+ cells may be administered in any suitable dosage following conditioning. Non-limiting examples of dosages are about 1×10⁵ CD34+ cells/kg of recipient to about 1×10⁷ CD34+ cells/kg (e.g., from about 2×10⁵ CD34+ cells/kg to about 9×10⁶ CD34+ cells/kg, from about 3×10⁵ CD34+ cells/kg to about 8×10⁶ CD34+ cells/kg, from about 4×10⁵ CD34+ cells/kg to about 7×10⁶ CD34+ cells/kg, from about 5×10⁵ CD34+ cells/kg to about 6×10⁶ CD34+ cells/kg, from about 5×10⁵ CD34+ cells/kg to about 1×10⁷ CD34+ cells/kg, from about 6×10⁵ CD34+ cells/kg to about 1×10⁷ CD34+ cells/kg, from about 7×10⁵ CD34+ cells/kg to about 1×10⁷ CD34+ cells/kg, from about 8×10⁵ CD34+ cells/kg to about 1×10⁷ CD34+ cells/kg, from about 9×10⁵ CD34+ cells/kg to about 1×10⁷ CD34+ cells/kg, and from about 1×10⁶ CD34+ cells/kg to about 1×10⁷ CD34+ cells/kg). Additional exemplary dosages are from about 1×10¹⁰ CD34+ cells/kg of recipient to about 1×10¹² CD34+ cells/kg (e.g., from about 2×10¹⁰ CD34+ cells/kg to about 9×10¹¹ CD34+ cells/kg, from about 3×10¹⁰ CD34+ cells/kg to about 8×10¹¹ CD34+ cells/kg, from about 4×10¹⁰ CD34+ cells/kg to about 7×10¹ CD34+ cells/kg, from about 5×10¹⁰ CD34+ cells/kg to about 6×10¹¹ CD34+ cells/kg, from about 5×10¹⁰ CD34+ cells/kg to about 1×10¹² CD34+ cells/kg, from about 6×10¹⁰ CD34+ cells/kg to about 1×10¹² CD34+ cells/kg, from about 7×10¹⁰ CD34+ cells/kg to about 1×10¹² CD34+ cells/kg, from about 8×10¹⁰ CD34+ cells/kg to about 1×10¹² CD34+ cells/kg, from about 9×10¹⁰ CD34+ cells/kg to about 1×10¹² CD34+ cells/kg, and from about 1×10¹¹ CD34+ cells/kg to about 1×10¹² CD34+ cells/kg), among others.

The cells and compositions described herein can be administered in an amount sufficient to improve one or more pathological features in Gaucher disease. Administration of the cells or compositions described herein may increase the quantity of anti-inflammatory microglia in the brain of the subject relative to the quantity of pro-inflammatory microglia in the brain of the subject, decrease the level of proinflammatory cytokines in the brain of the subject, increase the level of anti-inflammatory cytokines in the brain of the subject, improve the cognitive performance of the subject, improve the motor function of the subject, increase expression and/or activity of GBA in cells of the subject, reduce accumulation of glucocerebrosides in lysosomes of phagocytic cells, reduce splenomegaly, hepatomegaly, bone disease, thrombocytopenia, anemia, fatigue, one or more neurological symptoms (e,g., impaired olfaction, cognition, seizures, hypertonia, intellectual disability, apnea, dementia, and ocular muscle apraxia), and low blood platelet count in the subject. The numbers of pro-inflammatory and anti-inflammatory microglia may be assessed using ELISAs to compare the level of cytokines, chemokines, and other pro- and anti-inflammatory mediators in the cerebrospinal fluid (CSF) of subjects before and after treatment, by using PET imaging to view translocator protein (TSPO), a protein highly expressed in classically activated Pro-inflammatory microglia, before and after treatment, e.g., using TSPO radioligand ¹¹C-(R)-PK11195, or by analyzing the levels of pro-inflammatory- and anti-inflammatory-associated genes and proteins in a tissue sample using standard techniques, e.g., western blot analysis, immunohistochemical analyses, or quantitative RT-PCR. Cognition and motor function can be assessed using standard neurological tests before and after treatment, and monomeric and oligomeric α-synuclein can be detected in plasma and CSF using ELISA. GBA and/or SCARB2 may reduce one or more pro-inflammatory cytokines, e.g., IL-1p, tumor necrosis factor, iNOS, IL-6, or IL-12, chemokines, e.g., MHC II, CD86, CD68, CD11B, or Fcγ receptors, oligomeric α-synuclein levels, and/or TSPO signal by 10%, 15%, 20%, 25%, 30%, 35%, 40%, 45%, 50%, 55%, 60%, 65%, 70%, 75%, 80%, 85%, 90%, 95%, 100%, 200%, 500%, or more. GBA and/or SCARB2 may increase one or more anti-inflammatory cytokines, e.g., IL-25, IL-10 or TGF-β, chemokines, e.g., CD163, TREM2, Arg1, or CD206, cognitive function, motor function, and/or F¹⁸-dopa signal by 10%, 15%, 20%, 25%, 30%, 35%, 40%, 45%, 50%, 55%, 60%, 65%, 70%, 75%, 80%, 85%, 90%, 95%, 100%, 200%, 500%, or more. GBA and/or SCARB2 may also slow or prevent a decrease in cognitive function, motor function. GBA and/or SCARB2 may also slow the progression of organ-specific disorders, including splenomegaly, hepatomegaly, and bone disease. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of pluripotent cells expressing one or more GBA and/or SCARB2. The patient may be evaluated 1 month, 2 months, 3 months, 4 months, 5 months, 6 months or more following administration of the population of pluripotent cells depending on the route of administration used for treatment. Depending on the outcome of the evaluation, the patient may receive additional treatments.

### Kits

The compositions described herein can be provided in a kit for use in treating Gaucher disease. Compositions may include host cells described herein one or more agents (e.g., HSCs, iPSCs, ES cells, CD34+ cells, myeloid progenitor cells) that increase the expression and/or activity of GBA and/or SCARB2, and, optionally, may have disrupted endogenous GBA. Cells may be cryopreserved, e.g., in dimethyl sulfoxide (DMSO), glycerol, or another cryoprotectant. The kit can include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Generation of a CD34+ Hematopoietic Stem Cell Expressing Non-Secreted Glucocerebrosidase (GBA) and/or SCARB2

An exemplary method for making pluripotent cells (e.g., CD34+ cells) that express GBA and/or SCARB2 for use in the compositions and methods described herein is by way of transduction. Retroviral vectors (e.g., a lentiviral vector, alpharetroviral vector, or gammaretroviral vector) containing a promoter, such as the MND promoter, a signal peptide, such as the GBA and/or SCARB2 signal peptide, and the polynucleotide encoding GBA and/or SCARB2 can be engineered using standard techniques known in the art. If pluripotent cells that express GBA and SCARB2, are to be made, a bicistronic expression cassette can be used in which a F2A, P2A, E2A, T2A, or an IRES sequence is placed between the polynucleotide encoding GBA and the polynucleotide encoding SCARB2. After the retroviral vector is engineered, the retrovirus can be used to transduce pluripotent cells (e.g., iPS cells, ES cells, CD34+ HSCs) to generate a population of pluripotent cells that express GBA and/or SCARB2.

Additional exemplary methods for making pluripotent cells that express GBA and/or SCARB2 for use in the compositions and methods described herein is transfection. Using molecular biology techniques known in the art, plasmid DNA containing a promoter (e.g., an MND promoter), a signal peptide, such as the GBA and/or SCARB2 signal peptide, and the polynucleotide encoding GBA and/or SCARB2 can be produced. For example, the GBA gene and/or the SCARB2 gene may be amplified from a human cell line using PCR-based techniques known in the art, or the gene may be synthesized, for example, using solid-phase polynucleotide synthesis procedures. The GBA gene and/or the SCARB2 gene and promoter can then be ligated into a plasmid of interest, for example, using suitable restriction endonuclease-mediated cleavage and ligation protocols. If pluripotent cells that express GBA and/or SCARB2 are to be made, a bicistronic expression cassette can be used in which an IRES sequence or a 2A sequence (e.g., F2A, P2A, E2A, or T2A) is placed between the polynucleotide encoding GBA and the polynucleotide encoding SCARB2. After the plasmid DNA is engineered, the plasmid can be used to transfect the pluripotent cells (e.g., PS cells, ES cells, CD34+ HSCs) using, for example, electroporation or another transfection technique described herein to generate a population of pluripotent cells that express GBA and/or SCARB2. In both exemplary methods described herein, the GBA and/or SCARB2 may be expressed as a fusion protein. The fusion protein may contain GBA and/or SCARB2 and a glycosylation independent lysosomal targeting (GILT) tag. Exemplary GILT tags are muteins derived from human insulin-like growth factor II (IGF-II) having an amino acid sequence that is at least 70% identical to the amino acid sequence of mature human IGF-II. These IGF-II muteins have diminished binding activity for the insulin receptor relative to the affinity of naturally-occurring human IGF-II for the insulin receptor, are resistant to furin cleavage, and bind to the human cation-independent mannose-6-phosphate receptor in a mannose-6-phosphate-independent manner. The GILT tag facilitates delivery of the secreted GBA fusion protein to the lysosome.

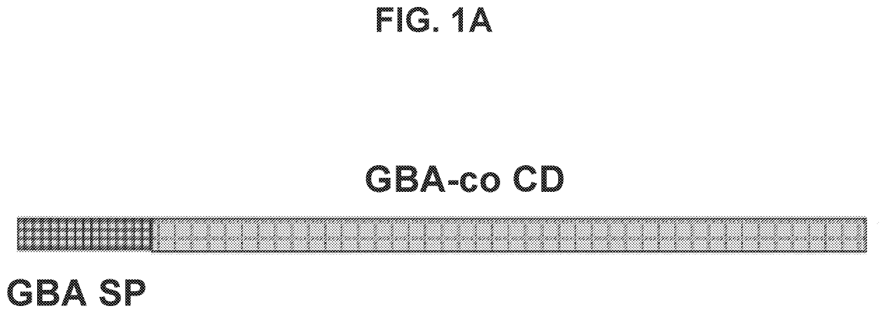

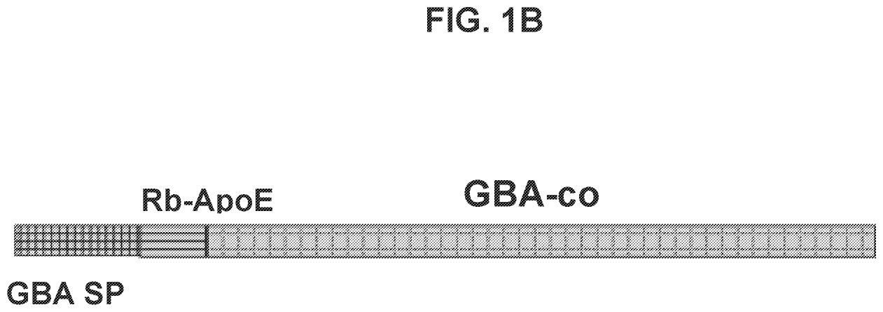

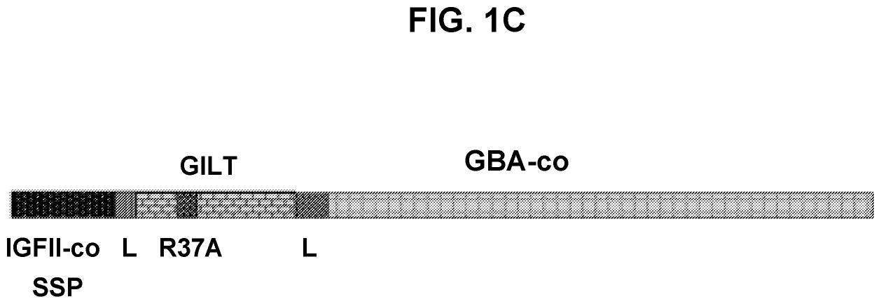

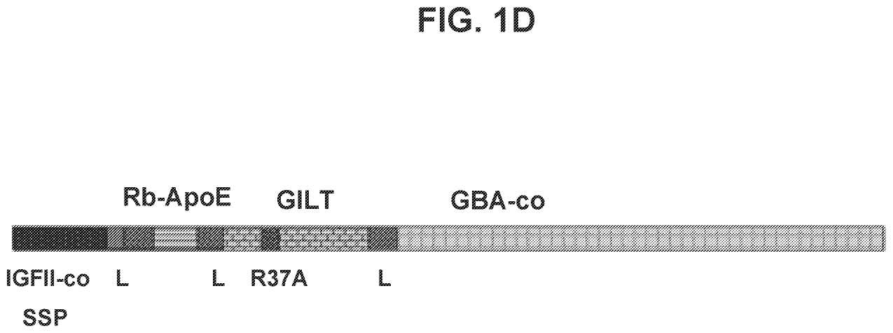

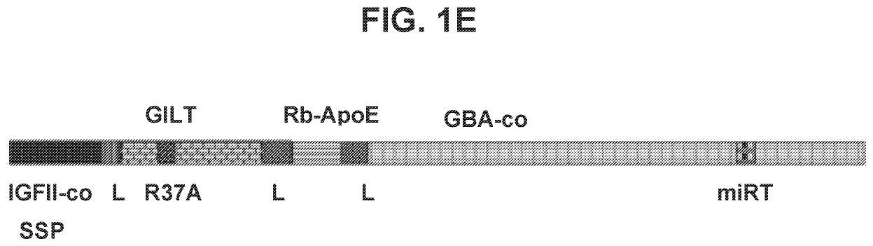

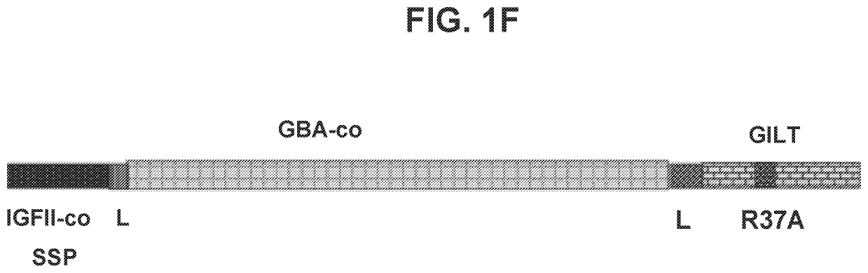

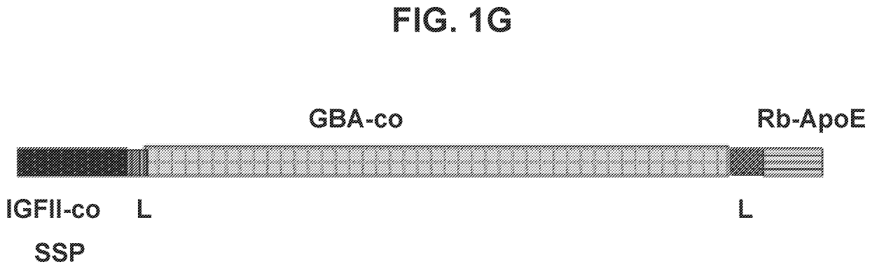

Exemplary constructs encoding GBA for use in conjunction with the compositions and methods described herein are shown in FIGS. 1A-1G.

### Example 2. Administration of a Population of Pluripotent Cells Expressing GBA and/or SCARB2 to a Patient Suffering from Gaucher Disease

According to the methods disclosed herein, a physician of skill in the art can treat a patient, such as a human patient, so as to reduce or alleviate symptoms of Gaucher disease. To this end, a physician of skill in the art can administer to the human patient a population of pluripotent cells, (e.g., CD34+ cells (such as ES cells, iPS cells, HSCs, or myeloid progenitor cells)) expressing GBA and/or SCARB2. The cells can be transduced or transfected ex vivo to express GBA and/or SCARB2 using techniques described herein or known in the art. The population of pluripotent cells expressing GBA and/or SCARB2 may be administered to the patient, for example, systemically (e.g., intravenously), directly to the central nervous system (CNS) (e.g., intracerebroventricularly or stereotactically), or directly into the bone marrow (e.g., intraosseously), to treat Gaucher disease. The cells can also be administered to the patient by multiple routes of administration, for example, intravenously and intracerebroventricularly. The cells are administered in a therapeutically effective amount, such as from 1×10⁶ cells/kg to 1×10¹² cells/kg or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more).

Before the population of pluripotent cells expressing GBA and/or SCARB2 is administered to the patient, one or more agents may be administered to the patient to ablate the patient's endogenous microglia and/or hematopoietic stem and progenitor cells, for example, busulfan, treosulfan, PLX3397, PLX647, PLX5622, and/or clodronate liposomes. Other methods of cell ablation well known in the art, such as irradiation, may be used alone or in combination with one or more of the aforementioned agents to ablate the patient's microglia and/or hematopoietic stem and progenitor cells. These agents and/or treatments may ablate endogenous microglia and/or hematopoietic stem and progenitor cells by at least 10% (e.g., at least 20%, 30%, 40%, 50%, 60%, 70%, 80%, 90%, 95%, or more), as assessed by PET imaging techniques known in the art. If the population of pluripotent cells expressing GBA and/or SCARB2 is administered to the patient after microglial ablation, the pluripotent cells can repopulate the brain, differentiating into microglia. The population of pluripotent cells expressing GBA and/or SCARB2 can be administered to the patient from, for example, 1 week to 1 month (e.g., 1 week, 2 weeks, 3 weeks, 4, weeks) or more after microglial ablation.

Following ablation of the patient's endogenous microglia and/or hematopoietic stem and progenitor cells, a population of CD34+ cells may be administered to the patient systemically (e.g., intravenously), or by bone marrow transplantation to reconstitute the bone marrow compartment. The number of CD34+ cells may be administered in any suitable dosage following conditioning. Non-limiting examples of dosages are about 1×10⁵ CD34+ cells/kg of recipient to about 1×10⁷ CD34+ cells/kg (e.g., from about 2×10⁵ CD34+ cells/kg to about 9×10⁶ CD34+ cells/kg, from about 3×10⁵ CD34+ cells/kg to about 8×10⁶ CD34+ cells/kg, from about 4×10⁵ CD34+ cells/kg to about 7×10⁶ CD34+ cells/kg, from about 5×10⁵ CD34+ cells/kg to about 6×10⁶ CD34+ cells/kg, from about 5×10⁵ CD34+ cells/kg to about 1×10⁷ CD34+ cells/kg, from about 6×10⁵ CD34+ cells/kg to about 1×10⁷ CD34+ cells/kg, from about 7×10⁵ CD34+ cells/kg to about 1×10⁷ CD34+ cells/kg, from about 8×10⁵ CD34+ cells/kg to about 1×10⁷ CD34+ cells/kg, from about 9×10⁵ CD34+ cells/kg to about 1×10⁷ CD34+ cells/kg, or from about 1×10⁶ CD34+ cells/kg to about 1×10⁷ CD34+ cells/kg, among others). Administration may occur prior to, or following administration of the pluripotent cells expressing GBA and/or SCARB2.

The population of pluripotent cells expressing GBA and/or SCARB2 can be administered to the patient in an amount sufficient to treat one or more of the pathological features of Gaucher disease. For example, the population of pluripotent cells expressing GBA and/or SCARB2 can be administered in an amount sufficient to increase the quantity of anti-inflammatory microglia in the brain of the patient relative to the quantity of pro-inflammatory microglia in the brain of the patient. The relative increase can be measured using conventional techniques known in the art, such as by performing an ELISA on patient CSF before and after treatment to assess the level of pro-inflammatory and anti-inflammatory cytokines secreted by pro-inflammatory and anti-inflammatory microglia at both time points. A standard neurological examination can also be performed by the physician before and after treatment to evaluate changes in cognitive performance and motor function. The patient may be evaluated, for example, 1 month, 2 months, 3 months, 4 months, 5 months, 6 months or more following administration of the population of pluripotent cells depending on the route of administration used for treatment. A finding of reduced pro-inflammatory cytokines, increased anti-inflammatory cytokines, and/or improved cognitive or motor function following administration of a population of pluripotent cells expressing GBA and/or SCARB2 provides an indication that the treatment has successfully treated Gaucher disease.

### Example 3. Disruption of Endogenous GBA in Pluripotent Cells Prior to Administration to a Patient Suffering from Gaucher Disease

In any of the methods disclosed herein, the pluripotent stem cells, such as CD34+ cells, (e.g., those expressing a transgene encoding GBA and/or SCARB2, or non-transgene containing CD34+ cells) may be treated to disrupt the endogenous GBA prior to administration to the patient. An exemplary method of disrupting endogenous GBA in pluripotent cells is using a CRISPR/Cas system (e.g., CRISPR/Cas9) with a GBA-specific guide RNA (gRNA) to induce one or more double-strand breaks (DSB). Following non-homologous end joining (NHEJ) to repair the DSB, the presence of newly-formed indel mutations will result in endogenous GBA disruption. Alternative methods for disruption of endogenous GBA by site-specifically cleaving genomic DNA prior to the incorporation of a GBA transgene in a pluripotent stem cell include the use of zinc finger nucleases (ZFNs) and transcription activator-like effector nucleases (TALENs). Unlike the CRISPR/Cas system, these enzymes do not contain a guiding polynucleotide to localize to a specific target sequence, but instead rely on internal DNA biding domains within the enzymes to mediate target specificity. In exemplary embodiments, the pluripotent cell is manipulated ex vivo by the nuclease to decrease or reduce the expression of endogenous GBA by 10% or more (e.g., 10%, 20%, 30%, 40%, 50%, 60%, 70%, 80%, 90%, 95% or more).

### Example 4. Generation of Mammalian Cell Lines Expressing Codon-Optimized GBA

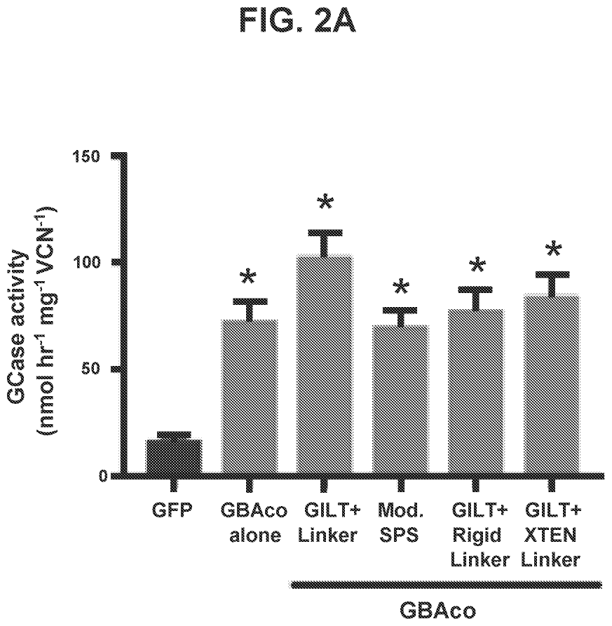

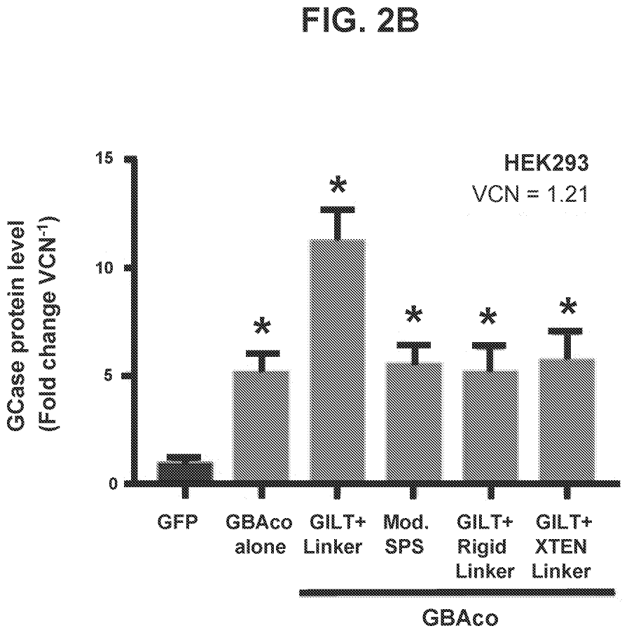

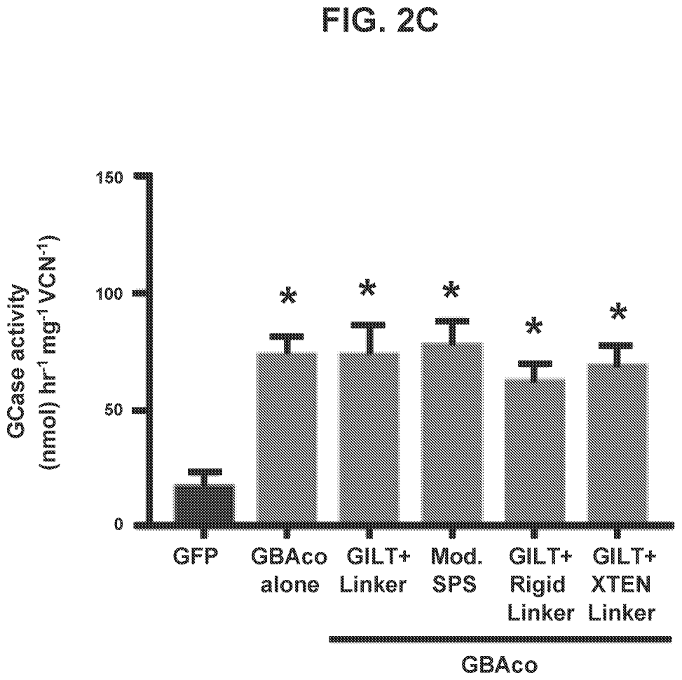

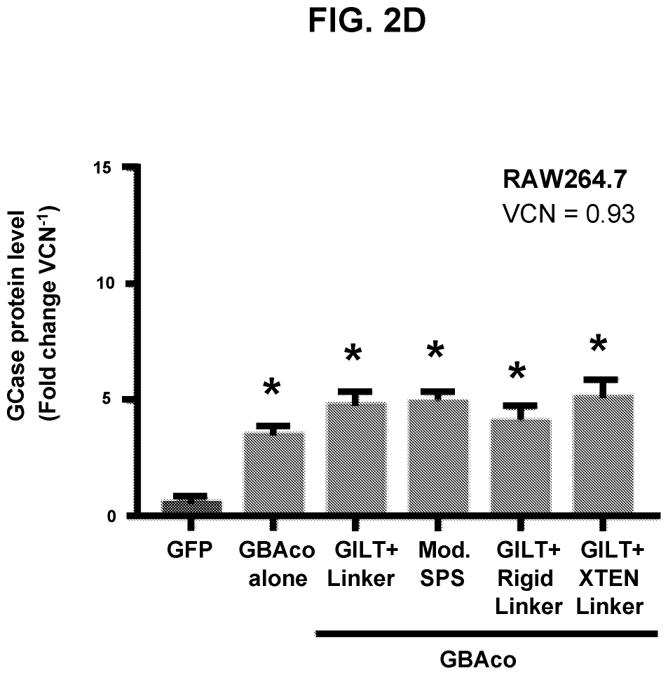

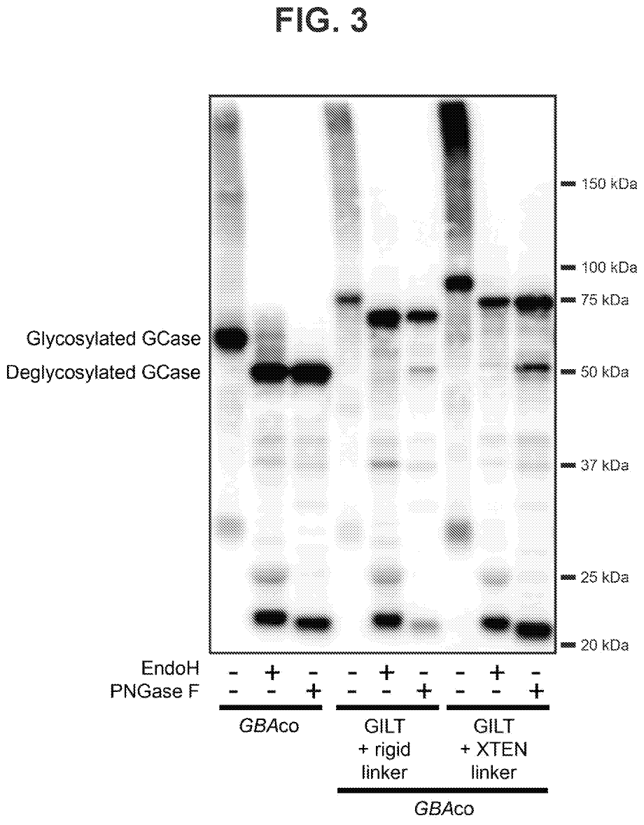

To assess the ability of lentivirally-encoded, codon-optimized GBA (GBAco) transgenes (e.g., GBAco transgenes having the codon-optimized nucleic acid sequence of any one of SEQ ID NO: 7-10) to stably express in mammalian cell lines, human HEK293T cells and murine RAW264.7 cells were transduced in vitro. Cells were transduced with one of six engineered constructs selected from the group including: 1) green fluorescent protein (GFP); 2) GBAco alone; 3) GBAco, a glycosylation independent lysosomal targeting (GILT) tag, and a peptide linker; 4) GBAco and a modified signal peptide sequence; 5) GBAco, a GILT tag, and a rigid peptide linker; or 6) GBAco, a GILT tag, and an XTEN linker. Protein samples were harvested post-transduction to measure GBA enzymatic activity and to quantify protein levels. GBA enzymatic activity was measured in using a 4-methylumbelliferyl β-D-glucopyranoside (4MUG) substrate, which is enzymatically converted by GBA to produce a fluorescent product 4-Methylumbelliferone (4MU). Lentiviral expression of GBAco constructs in either HEK293T (vector copy number (VCN)=1.21) or RAW264.7 (VCN=0.93) cells increased GBA enzymatic activity (FIGS. 2A, 2C) and protein levels (FIGS. 2B, 2D) relative to control GFP construct (p<0.05 ANOVA with Tukey post-hoc analysis, n=3 independent transductions). Furthermore, engineered GBA proteins were detected at the predicted molecular weights, suggesting that GILT and linker peptides were stably produced (FIG. 3).

Post-translational glycosylation at the N-terminus of GBA proteins (e.g., N-linked glycosylation) is critical for GBA function. Thus, engineered GBA proteins were tested for N-linked glycosylation in HEK293T cells in vitro. We assayed the electrophoretic mobility shift of the engineered GBA proteins after enzymatic digestion with either EndoH or PNGase F glycosidases that remove N-linked sugars from glycoproteins. Western blot analysis of cell lysates revealed increased electrophoretic mobility following de-glycosylation in all of the tested constructs (e.g., GBAco; GBAco, a GILT tag, and a rigid linker; or GBAco, a GILT tag, and an XTEN linker; FIG. 3), indicating that engineered GBA proteins are glycosylated in mammalian cell lines.

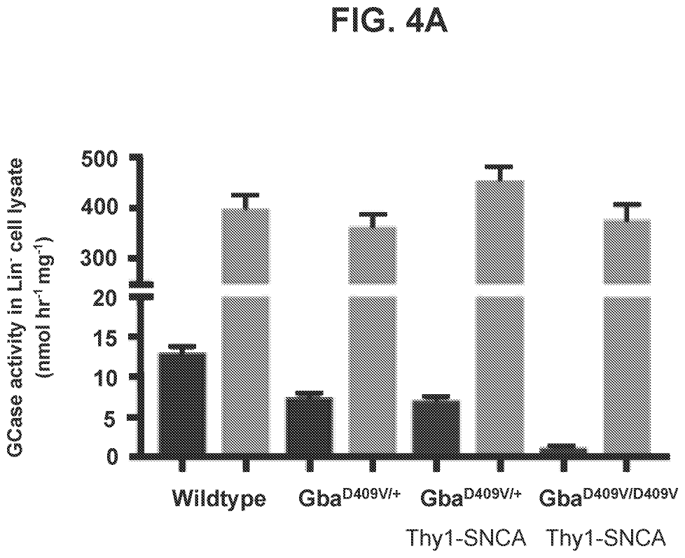

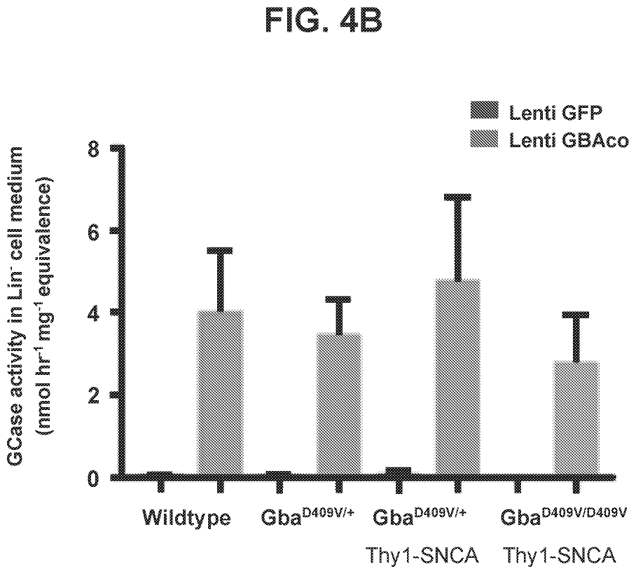

To determine whether engineered GBA constructs can be stably expressed in hematopoietic stem cells, lentivirally-encoded constructs were transduced into murine lineage negative (Lin⁻) hematopoietic stem cells isolated from the bone marrow of murine models of GBA deficiency (Gba^{D409V/+} n=3; Thy1-SNCA; Gba^{D409V/+} n=1; Thy1-SNCA; Gba^{D409V/D409V} n=2; wildtype (WT) n=2). Five days after transduction with either lentiviral constructs encoding GFP or GBAco (multiplicity of infection=80), Lin⁻ cells were >83% viable (assayed by trypan blue dye exclusion) and remained multipotent (>50 colony forming units). Enzymatic assays of Lin⁻ cells demonstrated that the heterozygous and homozygous Gba mutations reduced GBA activity by 43% and 92%, respectively, in the absence of GBA transgenes (WT: 13.04±0.644 nmol hr⁻¹ mg⁻¹; Gba^{D409V/+}: 7.49±0.293 nmol hr⁻¹ mg⁻¹; Thy1-SNCA; Gba^{D409V/+}: 7.14±0.252 nmol hr⁻¹ mg⁻¹; Thy1-SNCA; Gba^{D409V/D409V}: 1.20±0.114 nmol hr⁻¹ mg⁻¹; p<0.001, ANOVA with Tukey post-hoc analysis, FIG. 4A). Importantly, GBAco transduction of Lin⁻ cells significantly increased GBA enzymatic activity to similar levels across all tested GBAco constructs relative to GFP control (p<0.001, ANOVA with Tukey post-hoc analysis; FIG. 4A). Furthermore, GBAco expression led to the detection of GBA activity in conditioned media from the Lin⁻ cells by Western blot (FIG. 4B). Combined, these findings demonstrate that lentiviral GBAco constructs produce a functional GBA enzyme in hematopoietic stem cells (e.g., murine Lin⁻ cells) and can rescue GBA activity and expression levels in murine models of GBA deficiency.

### Example 5. Enhanced GBA Activity in Murine Macrophages Transduced with a Bicistronic Lentiviral Vector Encoding GBA and SCARB2

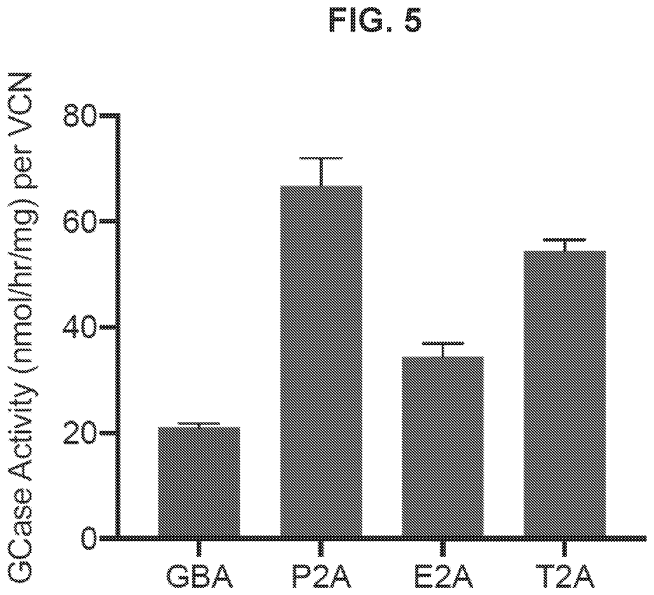

To assess the ability of lentivirally-encoded, bicistronic transgenes (e.g., transgenes encoding GBA and SCARB2 separated by a P2A, E2A, or T2A self-cleaving peptides) to stably express GBA in mammalian cell lines, lentiviral vectors were transduced into a RAW264.7 murine macrophage cell line. Four days after transduction, enzymatic assays were performed to assess GBA enzymatic activity in transduced cells. Vector copy numbers (VCN) were determined using quantitative polymerase chain reaction (qPCR). GBA activity per VCN were determined by dividing GBA activity by from the enzymatic assay by the values obtained from the qPCR experiments. These results demonstrated that GBAco co-transduction with SCARB2 significantly increased GBA enzymatic activity to similar levels across all tested GBAco constructs (FIG. 5).

Accordingly, co-administration of agents that increase the expression and/or activity of GBA and SCARB2 may serve as a potential therapeutic avenue for the treatment of Gaucher disease.

### Example 6. Ex Vivo Lentiviral Gene Therapy Leads to Substrate Reduction in the Lung Methods

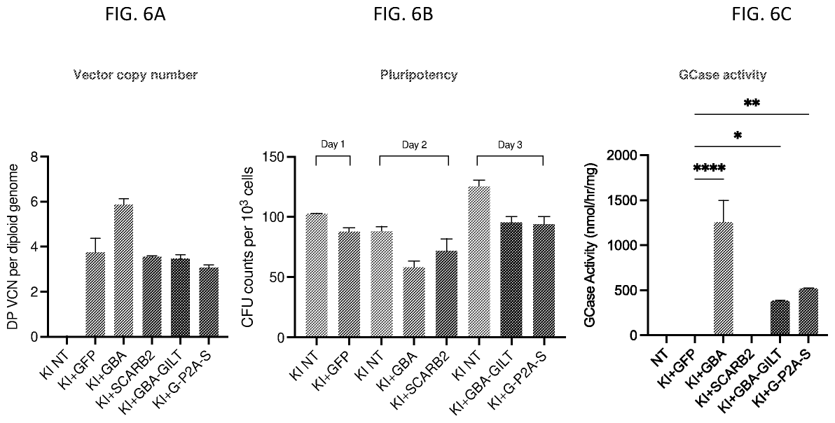

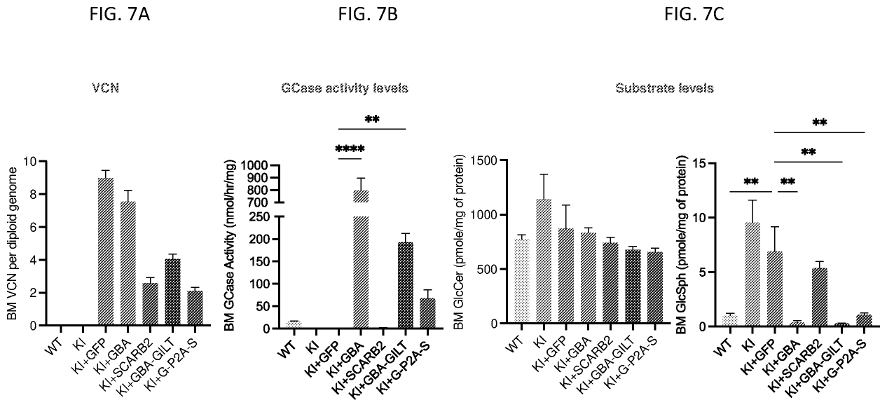

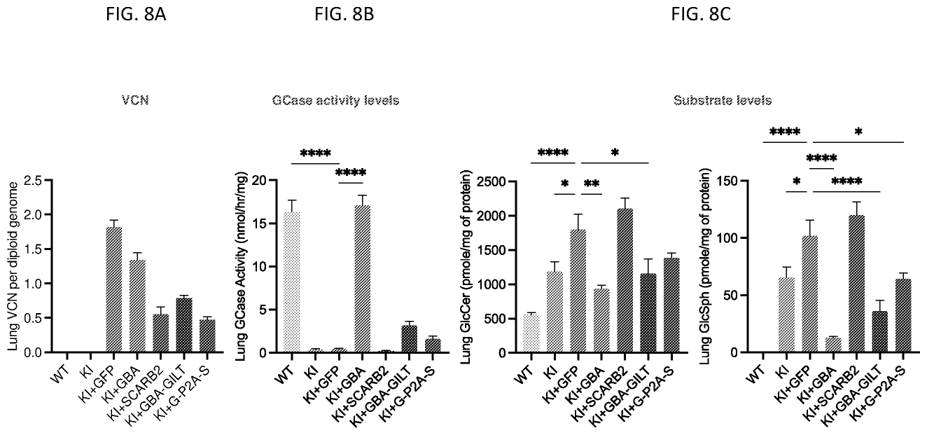

Gba mutant donor mice were sacrificed, bone marrow cells were harvested, and lineage negative (Lin−) hematopoietic stem/progenitor cells were isolated. Lin− were then transduced with a lentiviral vector encoding GFP, human GBA, human SCARB2, human GBA fused with the GILT tag, or human GBA and human SCARB2 (separated by a P2A sequence). These cells were collected and washed approximately 16 hours later and transplanted into busulfan-conditioned Gba mutant mice (Gba D409V knock-in or KI). Cells were assessed for VCN, pluripotency, and GCase activity (FIGS. 6A-6C). Approximately 16 weeks later, mice were sacrificed, and bone marrow (FIGS. 7A-7C) and lungs (FIGS. 8A-8C) were collected and analyzed for vector copy number per genome by qPCR, GCase enzymatic activity was assessed by 4-MU assay, and substrate levels were assessed by mass spectrometry.

### Results

All animals treated with GBA encoding vectors (GBA alone, GBA-GILT, and GBA-P2A-SCARB2) exhibited a significantly higher level of activity than wild-type and GFP-treated Gba KI mice in the bone marrow (FIGS. 7A-7C). Furthermore, a significant reduction in glucosylsphingosine levels was observed in these treated groups compared to GFP-treated Gba KI mice. Similarly, an increase in GCase activity levels and reduction in glucosylsphingosine accumulation in the lung was observed (FIGS. 8A-8C). The difference in GCase activity between the groups can at least partially be attributed to the VCN differences.

Accordingly, co-administration of agents that increase the expression and/or activity of GBA and SCARB2 may serve as a potential therapeutic avenue for the treatment of Gaucher disease.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A method of treating a subject diagnosed as having or at risk of developing Gaucher disease, the method comprising providing to the subject one or more agents that collectively increase expression and/or activity of β-glucocerebrosidase (GBA) and scavenger receptor class B member 2 (SCARB2).

2. The method of claim 1, wherein the one or more agents comprise a first agent that increases expression and/or activity of GBA and a second agent that increases expression and/or activity of SCARB2; optionally, wherein the first agent comprises (i) one or more polynucleotides comprising a transgene that encodes a GBA protein, (ii) one or more interfering RNA (RNAi) molecules that collectively increase expression and/or activity of the GBA protein, (iii) one or more polynucleotides encoding the one or more RNAi molecules that collectively increase expression and/or activity of the GBA protein, (iv) a GBA protein, or (v) one or more small molecules that collectively increase expression and/or activity of the GBA protein, and wherein the second agent comprises (vi) one or more polynucleotides comprising a transgene that encodes a SCARB2 protein, (vii) one or more RNAi molecules that collectively increase expression and/or activity of the SCARB2 protein, (viii) one or more polynucleotides encoding the one or more RNAi molecules that collectively increase expression and/or activity of the SCARB2 protein, (ix) a SCARB2 protein, (x) one or more small molecules that collectively increase expression and/or activity of the SCARB2 protein.

3. The method of claim 2, wherein the GBA protein has an amino acid sequence that is at least 85% identical to the amino acid sequence of SEQ ID NO: 1.

4. The method of claim 3, wherein the GBA protein has an amino acid sequence that is at least 90% identical to the amino acid sequence of SEQ ID NO: 1; optionally, wherein the GBA has an amino acid sequence that is at least 95% identical to the amino acid sequence of SEQ ID NO: 1; optionally, wherein the GBA has the amino acid sequence of SEQ ID NO: 1.

5. The method of claim 2, wherein the GBA has an amino acid sequence that is at least 85% identical to the amino acid sequence of SEQ ID NO: 5.

6. The method of claim 5, wherein the GBA has an amino acid sequence that is at least 90% identical to the amino acid sequence of SEQ ID NO: 5; optionally, wherein the GBA has an amino acid sequence that is at least 95% identical to the amino acid sequence of SEQ ID NO: 5; optionally; wherein the GBA has the amino acid sequence of SEQ ID NO: 5.

7. The method of any one of claims 2-6 wherein the transgene encoding GBA has a nucleic acid sequence that is at least 85% identical to the nucleic acid sequence of SEQ ID NO: 6.

8. The method of claim 7, wherein the transgene encoding GBA has a nucleic acid sequence that is at least 90% identical to the nucleic acid sequence of SEQ ID NO: 6; optionally, wherein the transgene encoding GBA has a nucleic acid sequence that is at least 95% identical to the nucleic acid sequence of SEQ ID NO: 6; optionally, wherein the transgene encoding GBA has the nucleic acid sequence of SEQ ID NO: 6.

9. The method of any one of claims 2-6, wherein the transgene encoding GBA has a nucleic acid sequence that is at least 85% identical to the nucleic acid sequence of SEQ ID NO: 7.

10. The method of claim 9, wherein the transgene encoding GBA has a nucleic acid sequence that is at least 90% identical to the nucleic acid sequence of SEQ ID NO: 7; optionally, wherein the transgene encoding GBA has a nucleic acid sequence that is at least 95% identical to the nucleic acid sequence of SEQ ID NO: 7; optionally, wherein the transgene encoding GBA has the nucleic acid sequence of SEQ ID NO: 7.

11. The method of any one of claims 2-6, wherein the transgene encoding GBA has a nucleic acid sequence that is at least 85% identical to the nucleic acid sequence of SEQ ID NO: 11.

12. The method of claim 11, wherein the transgene encoding GBA has a nucleic acid sequence that is at least 90% identical to the nucleic acid sequence of SEQ ID NO: 11; optionally, wherein the transgene encoding GBA has a nucleic acid sequence that is at least 95% identical to the nucleic acid sequence of SEQ ID NO: 11; optionally, wherein the transgene encoding GBA has the nucleic acid sequence of SEQ ID NO: 11.

13. The method of any one of claims 2-12, wherein the GBA comprises a signal peptide, wherein the signal peptide has an amino acid sequence that is at least 85% identical to the amino acid sequence of SEQ ID NO: 12.

14. The method of claim 13, wherein the signal peptide has an amino acid sequence that is at least 90% identical to the amino acid sequence of SEQ ID NO: 12; optionally, wherein the signal peptide has an amino acid sequence that is at least 95% identical to the amino acid sequence of SEQ ID NO: 12; optionally, wherein the signal peptide has the amino acid sequence of SEQ ID NO: 12.

15. The method of claim 2-12, wherein the GBA comprises a signal peptide, wherein the signal peptide is encoded by a polynucleotide that is at least 85% identical to the nucleic acid sequence of SEQ ID NO: 13.

16. The method of claim 15, wherein the signal peptide is encoded by a polynucleotide that is at least 90% identical to the nucleic acid sequence of SEQ ID NO: 13; optionally, wherein the signal peptide is encoded by a polynucleotide that is at least 95% identical to the nucleic acid sequence of SEQ ID NO: 13; optionally, wherein the signal peptide is encoded by a polynucleotide having the nucleic acid sequence of SEQ ID NO: 13.

17. The method of any one of claims 2-16, wherein the SCARB2 has an amino acid sequence that is at least 85% identical to the amino acid sequence of SEQ ID NO: 14.

18. The method of claim 17, wherein the SCARB2 has an amino acid sequence that is at least 90% identical to the amino acid sequence of SEQ ID NO: 14; optionally, wherein the SCARB2 has an amino acid sequence that is at least 95% identical to the amino acid sequence of SEQ ID NO: 14; optionally, wherein the SCARB2 has an amino acid sequence of SEQ ID NO: 14.

19. The method of claim any one of claims 2-16, wherein the SCARB2 has an amino acid sequence that is at least 85% identical to the amino acid sequence of SEQ ID NO: 15.

20. The method of claim 19, wherein the SCARB2 has an amino acid sequence that is at least 90% identical to the amino acid sequence of SEQ ID NO: 15; optionally, wherein the SCARB2 has an amino acid sequence that is at least 95% identical to the amino acid sequence of SEQ ID NO: 15; optionally, wherein the SCARB2 has an amino acid sequence of SEQ ID NO: 15.

21. The method of claim of any one of claims 2-20, wherein the polynucleotide encoding SCARB2 has a nucleic acid sequence that is at least 85% identical to the nucleic acid sequence of SEQ ID NO: 16.

22. The method of claim 21, wherein the polynucleotide encoding SCARB2 has a nucleic acid sequence that is at least 90% identical to the nucleic acid sequence of SEQ ID NO: 16; optionally, wherein the polynucleotide encoding SCARB2 has a nucleic acid sequence that is at least 95% identical to the nucleic acid sequence of SEQ ID NO: 16; optionally, wherein the polynucleotide encoding SCARB2 has a nucleic acid sequence of SEQ ID NO: 16.

23. The method of any one of claims 2-20, wherein the polynucleotide encoding SCARB2 has a nucleic acid sequence that is at least 85% identical to the nucleic acid sequence of SEQ ID NO: 17.

24. The method of claim 23, wherein the polynucleotide encoding SCARB2 has a nucleic acid sequence that is at least 90% identical to the nucleic acid sequence of SEQ ID NO: 17; optionally, wherein the polynucleotide encoding SCARB2 has a nucleic acid sequence that is at least 95% identical to the nucleic acid sequence of SEQ ID NO: 17; optionally, wherein the polynucleotide encoding SCARB2 has a nucleic acid sequence of SEQ ID NO: 17.

25. The method of claim 2-24, wherein the SCARB2 comprises a signal peptide, wherein the signal peptide has an amino acid sequence that is at least 70% identical to the amino acid sequence of SEQ ID NO: 49; optionally, wherein the signal peptide has an amino acid sequence that is at least 75% identical to the amino acid sequence of SEQ ID NO: 49; optionally, wherein the signal peptide has an amino acid sequence that is at least 80% identical to the amino acid sequence of SEQ ID NO: 49; optionally, wherein the signal peptide has an amino acid sequence that is at least 85% identical to the amino acid sequence of SEQ ID NO: 49; optionally, wherein the signal peptide has an amino acid sequence that is at least 90% identical to the amino acid sequence of SEQ ID NO: 49; optionally, wherein the signal peptide has an amino acid sequence that is at least 95% identical to the amino acid sequence of SEQ ID NO: 49; optionally, wherein the signal peptide has an amino acid sequence of SEQ ID NO: 49.

26. The method of any one of claims 2-25, wherein the SCARB2 is a GBA-binding domain of SCARB2.

27. The method of claim 26, wherein the GBA-binding domain of the SCARB2 protein has an amino acid sequence that is at least 85% identical to the amino acid sequence of SEQ ID NO: 18; optionally, wherein the GBA-binding domain of the SCARB2 protein has an amino acid sequence that is at least 90% identical to the amino acid sequence of SEQ ID NO: 18; optionally, wherein the GBA-binding domain of the SCARB2 protein has an amino acid sequence that is at least 95% identical to the amino acid sequence of SEQ ID NO: 18; optionally, wherein the GBA-binding domain of the SCARB2 protein has an amino acid sequence of SEQ ID NO: 18.

28. The method of claim 26, wherein the GBA-binding domain of the SCARB2 protein has an amino acid sequence that is at least 85% identical to the amino acid sequence of SEQ ID NO: 19; optionally, wherein the GBA-binding domain of the SCARB2 protein has an amino acid sequence that is at least 90% identical to the amino acid sequence of SEQ ID NO: 19; optionally, wherein the GBA-binding domain of the SCARB2 protein has an amino acid sequence that is at least 95% identical to the amino acid sequence of SEQ ID NO: 19; optionally, wherein the GBA-binding domain of the SCARB2 protein has an amino acid sequence of SEQ ID NO: 19.

29. The method of claim any one of claims 2-28 wherein the GBA and/or SCARB2 is a fusion protein comprising GBA or SCARB2 and a glycosylation independent lysosomal targeting (GILT) tag.

30. The method of claim 29, wherein the GILT tag comprises a human IGF-II mutein having an amino acid sequence that is at least 70% identical to the amino acid sequence of mature human IGF-II (SEQ ID NO: 22), and having diminished binding affinity for the insulin receptor relative to the affinity of naturally-occurring human IGF-II for the insulin receptor, wherein the IGF-II mutein is resistant to furin cleavage and binds to the human cation-independent mannose-6-phosphate receptor in a mannose-6-phosphate-independent manner.

31. The method of claim 30, wherein the IGF-II mutein comprises a mutation within a region corresponding to amino acids 30-40 of SEQ ID NO: 22, and wherein the mutation abolishes at least one furin protease cleavage site.

32. The method of claim 31, wherein the mutation is an amino acid substitution, deletion, and/or insertion.

33. The method of claim 32, wherein the mutation is an Ala amino acid substitution at a position corresponding to Arg37 of SEQ ID NO: 22.

34. The method of claim 32, wherein the mutation is a deletion or replacement of amino acid residues corresponding to positions selected form the group consisting of 31-40, 32-40, 33-40, 34-40, 30-39, 31-39, 32-39, 34-37, 33-39, 35-39, 36-39, 37-40, 34-40 of SEQ ID NO: 22, and combinations thereof.

35. The method of any one of claims 29-34, wherein the GBA fusion protein and/or the SCARB2 fusion protein comprises a receptor-binding (Rb) domain of apolipoprotein E (ApoE).

36. The method of claim 35, wherein the Rb domain comprises a portion of ApoE having the amino acid sequence of residues 25-185, 50-180, 75-175, 100-170, 125-160, or 130-150 of SEQ ID NO: 29.

37. The method of claim 35, wherein the Rb domain comprises a region having at least 70% sequence identity to the amino acid sequence of residues 159-167 of SEQ ID NO: 29.

38. The method of claim any one of claims 2-37 wherein the GBA protein or the SCARB2 protein is a fusion protein comprising GBA or SCARB2 and a cell-penetrating peptide (CPP).

39. The method of claim 38, wherein the CPP has an amino acid sequence having at least 85% sequence identity to the amino acid sequence of any one of SEQ ID NOs: 30-48; optionally, wherein the CPP has an amino acid sequence having at least 90% sequence identity to the amino acid sequence of any one of SEQ ID NOs: 30-48; optionally, wherein the CPP has an amino acid sequence having at least 95% sequence identity to the amino acid sequence of any one of SEQ ID NOs: 30-48; optionally, wherein the CPP has an amino acid sequence having the amino acid sequence of any one of SEQ ID NOs: 30-48.

40. The method of any one of claims 2-39, wherein the transgene encoding GBA and/or SCARB2 further comprises a microRNA (miRNA) targeting sequence in the 3′-UTR.

41. The method of claim 40, wherein the miRNA targeting sequence is a miR-126 targeting sequence.

42. The method of any one of claims 2-41, wherein the GBA and/or the SCARB2 penetrates the blood brain barrier (BBB) in the subject.

43. The method of any one of claims 2-42, wherein the one or more RNAi molecules comprise short interfering RNA (siRNA), short hairpin RNA (shRNA), and/or microRNA (miRNA).

44. The method of any one of claims 2-43, wherein the one or more polynucleotides are provided to the subject by administering to the subject a composition comprising a population of cells that together contain nucleic acids encoding the GBA and/or SCARB2 protein.

45. The method of claim 44, wherein the population is a uniform population of cells that contain nucleic acids encoding the proteins or a heterogeneous population of cells that together contain nucleic acids encoding the GBA and/or SCARB2 protein.

46. The method of claim 44 or 45, wherein the cells are pluripotent cells or multipotent cells.

47. The method of claim 46, wherein the multipotent cells are CD34+ cells.

48. The method of claim 47, wherein the CD34+ cells are hematopoietic stem cells (HSCs) or myeloid progenitor cells (MPCs).

49. The method of claim 46, wherein the pluripotent cells are embryonic stem cells (ESCs) or induced pluripotent stem cells (iPSCs).

50. The method of claim 44 or 45, wherein the cells are blood lineage progenitor cells (BLPCs), microglial progenitor cells, monocytes, macrophages, or microglia.

51. The method of claim 50, wherein the BLPCs are monocytes.

52. The method of any one of claims 1-51, wherein a population of endogenous hematopoietic cells in the subject has been ablated prior to administration of the one or more agents to the subject, optionally wherein the hematopoietic cells are CD34+ cells, BLPCs, microglial progenitor cells, monocytes, macrophages, or microglia.

53. The method of any one of claims 1-51, the method comprising ablating a population of endogenous hematopoietic cells in the subject prior to administering the one or more agents to the subject, optionally wherein the hematopoietic cells are CD34+ cells, BLPCs, microglial progenitor cells, monocytes, macrophages, or microglia.

54. The method of claim 52 or 53, wherein the microglia are ablated using an agent selected from the group consisting of busulfan, PLX3397, PLX647, PLX5622, treosulfan, and clodronate liposomes, by radiation therapy, or a combination thereof.

55. The method of any one of claims 1-54, wherein the one or more agents is administered systemically to the subject.

56. The method of claim 55, wherein the one or more agents is administered to the subject by way of intravenous injection.

57. The method of any one of claims 1-54, wherein the one or more agents is administered directly to the central nervous system of the subject.

58. The method of claim 57, wherein the one or more agents is administered to the subject by way of intracerebroventricular injection, stereotactic injection, or a combination thereof.

59. The method of any one of claims 1-54, wherein the one or more agents is administered directly to the bone marrow of the subject.

60. The method of claim 59, wherein the one or more agents is administered to the subject by way of intraosseous injection.

61. The method of any one of claims 44-60, wherein the cells are autologous cells or allogeneic cells.

62. The method of any one of claims 44-61, wherein the cells are transfected or transduced ex vivo to express the GBA and/or SCARB2.

63. The method of claim 62, wherein the cells are transduced with a viral vector selected from the group consisting of an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus.

64. The method of claim 63, wherein the viral vector is a Retroviridae family viral vector.

65. The method of claim 64, wherein the Retroviridae family viral vector is a lentiviral vector.

66. The method of claim 64, wherein the Retroviridae family viral vector is an alpharetroviral vector.

67. The method of claim 64, wherein the Retroviridae family viral vector is a gammaretroviral vector.

68. The method of any one of claims 63-67, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

69. The method of claim 63, wherein the viral vector is a pseudotyped viral vector.

70. The method of claim 69, wherein the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

71. The method of any one of claims 62-70, wherein the pluripotent cells are transduced to express the GBA and SCARB2 from separate monocistronic expression cassettes.

72. The method of any one of claims 62-70, wherein the pluripotent cells are transduced to express the GBA and SCARB2 from a polycistronic expression cassette.

73. The method of claim 72, wherein the pluripotent cells are transduced to express the GBA and SCARB2 from a bicistronic expression cassette.

74. The method of claim 72 or 73, wherein the polycistronic expression cassette comprises an internal ribosomal entry site (IRES) positioned between a polynucleotide encoding the GBA and a polynucleotide encoding the SCARB2.

75. The method of claim 72 or 73, wherein the polycistronic expression cassette comprises a 2A polynucleotide positioned between a polynucleotide encoding the GBA and a polynucleotide encoding the SCARB2.

76. The method of claim 75, wherein the 2A polynucleotide comprises a F2A, P2A, E2A, or T2A polynucleotide.

77. The method of any one of claims 2-76, wherein one or more of the polynucleotides comprises a transgene encoding one or more of the proteins operably linked to a ubiquitous promoter, a cell lineage-specific promoter, or a synthetic promoter.

78. The method of claim 77, wherein the ubiquitous promoter is selected from the group consisting of an elongation factor 1-alpha (EF1α) promoter, phosphoglycerate kinase 1 (PGK) promoter, or EF1α promoter containing elements of locus control region of the β-globin gene containing regions of erythroid-specific DNase I hypersensitivity (HS) regions 2, 3, and 4 (β-LCR(HS4,3,2)-EFS promoter).

79. The method of claim 77, wherein the cell lineage-specific promoter is selected from the group consisting of a CD68 molecule (CD68) promoter, CD11 b molecule (CD11b) promoter, C-X3-C motif chemokine receptor 1 (CX3CR1) promoter, allograft inflammatory factor 1 promoter (AIF1) promoter, purinergic receptor P2Y12 (P2Y12) promoter, transmembrane protein 119 (TMEM119) promoter, or colony stimulating factor 1 receptor (CSF1R) promoter.

80. The method of claim 77, wherein the synthetic promoter is a Myeloproliferative Sarcoma Virus Enhancer, Negative Control Region Deleted, dl587rev Primer-Binding Site Substituted (MND) promoter.

81. The method of claim 80, wherein the MND promoter comprises a polynucleotide having at least 85% sequence identity to the nucleic acid sequence of SEQ ID NO: 20.

82. The method of claim 80, wherein the MND promoter comprises a polynucleotide having at least 85% sequence identity to the nucleic acid sequence of SEQ ID NO: 21.

83. The method of any one of claims 1-82, wherein the Gaucher disease is associated with one or more mutations in the GBA gene.

84. The method of claim 83, wherein the one or more mutations in the GBA gene comprise a p.N370S substitution, p.R463C substitution, p.L444P substitution, p.D409H substitution, p.R463C substitution, p.R496H substitution, p. F252I substitution, p.A456P substitution, p.V460V substitution, p.V394L, p.E326K substitution, p.G377S substitution, p.N188S substitution, c.84insG insertion, c.84dupG (84GG) duplication, c.115+1 G>A substitution, or c.IVS2DS+1 G-A splice site mutation.

85. The method of any one of claims 1-84, wherein the Gaucher disease is Type 1 Gaucher disease.

86. The method of claim 85, wherein the subject has a confirmed diagnosis of Type 1 Gaucher disease based on genotyping, deficient GBA activity in the blood of the subject, and/or clinical phenotype.

87. The method of claim 86, wherein the deficient GBA activity in the subject is defined as activity that is equal to or greater than 15% of activity of GBA in a control reference patient not diagnosed as having Gaucher disease.

88. The method of any one of claims 1-84, wherein the Gaucher disease is Type 2 Gaucher disease.

89. The method of any one of claims 1-84, wherein the Gaucher disease is Type 3 Gaucher disease.

90. The method of any one of claims 1-89, wherein the Gaucher disease is associated with one or more mutations in the SCARB2 gene.

91. The method of claim 90, wherein the one or more mutations in the SCARB2 gene comprise a p.Q471G substitution, p.H363N substitution, p.Q288Ter nonsense mutation, p.W178Ter nonsense mutation, p.W146fs frameshift mutation, p.Glu420fs frameshift mutation, g.76168478T>G transversion mutation, g.1239+1G-T splice site mutation, or g.76168401dup splice site mutation p.Q288Ter nonsense mutation, p.W178Ter nonsense mutation, p.W146fs frameshift mutation, p.Glu420fs frameshift mutation, g.76168478T>G transversion mutation, g.1239+1G-T splice site mutation, or g.76168401dup splice site mutation.

92. The method of any one of claims 1-91, wherein the subject is a human.

93. The method of any one of claims 1-92, wherein the subject has undergone enzyme replacement therapy (ERT) comprising a total monthly dose of GBA ERT that is greater than 30 U/kg and less than 120 U/kg for 24 or more consecutive months at a time of treatment with the one or more agents.

94. The method of claim 93, wherein the subject has received a biweekly dose of GBA ERT greater than or equal to 15 U/kg and less than or equal to 60 U/kg.

95. The method of claim 93, wherein the subject has received a weekly dose of GBA ERT greater than or equal to 7.5 U/kg and less than or equal to 30 U/kg.

96. The method of any one of claims 1-95, wherein the subject has received substrate reduction therapy (SRT) for Gaucher disease during the 24 months immediately preceding treatment with the one or more agents.

97. The method of any one of claims 1-95, wherein the subject has not received SRT for Gaucher disease during the 24 months immediately preceding treatment with the one or more agents.


SEQUENCE LISTING (49 sequences)

SEQ ID NO: 1 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 2 - protein, 621 aa, Artificial Sequence
  Synthetic Construct
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQGGGGAGGGGAGGGGAGGGGAGGGPSLCGGELVDTLQFVCGDRGFYFSRPASRVSARSRGIVEECCFRSCDLALLETYCATPAKSE

SEQ ID NO: 3 - protein, 627 aa, Artificial Sequence
  Synthetic Construct
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQGAPGGGSPAPAPTPAPAPTPAPAGGGPSGAPLCGGELVDTLQFVCGDRGFYFSRPASRVSARSRGIVEECCFRSCDLALLETYCATPAKSE

SEQ ID NO: 4 - protein, 652 aa, Artificial Sequence
  Synthetic Construct
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQGAPGGSPAGSPTSTEEGTSESATPESGPGTSTEPSEGSAPGSPAGSPTSTGPSGAPLCGGELVDTLQFVCGDRGFYFSRPASRVSARSRGIVEECCFRSCDLALLETYCATPAKSE

SEQ ID NO: 5 - protein, 521 aa, Artificial Sequence
  Synthetic Construct
MGIPMGKSMLVLLTFLAFASCCIAARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 6 - DNA, 2275 bp, Homo sapiens
  misc_feature (2005)..(2005): n is a, c, g, or t
gctaacctagtgcctatagctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggtttgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgcccagggctggaggcccctagaaaaagatcagtaagccccagtgtccccccagcccccatgcttatgtgaacatgcgctgtgtgctgcttgctttggaaactngcctgggtccaggcctagggtgagctcactgtccgtacaaacacaagatcagggctgagggtaaggaaaagaagagactaggaaagctgggcccaaaactggagactgtttgtctttcctagagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccca

SEQ ID NO: 7 - DNA, 1611 bp, Artificial Sequence
  Synthetic Construct
atggaattcagcagccccagccgggaggaatgccccaagcccctgagcagagtgtccatcatggccggatctctgaccggactgctgctgctgcaagccgtgtcttgggccagcggcgccagaccttgcatccccaagagcttcggctacagcagcgtcgtgtgcgtgtgcaacgccacctactgcgacagcttcgacccccctacctttcccgccctgggcaccttcagcagatacgagagcacccgcagcggcagacggatggaactgagcatgggccccatccaggccaatcacaccggaaccggcctgctgctgaccctgcagcccgagcagaaattccagaaagtgaagggcttcggcggagccatgacagacgccgctgccctgaacatcctggccctgagcccccctgctcagaatctgctgctcaagagctacttcagcgaggaaggcatcggctacaacatcatccgggtgcccatggctagctgcgacttcagcatccggacctacacctacgccgacacccccgacgacttccagctgcacaacttcagcctgcccgaagaggacaccaagctgaagatccccctgatccacagagccctgcagctggcccagaggcctgtgtctctgctggctagcccttggaccagccccacctggctgaaaaccaacggagccgtgaacggcaagggcagcctgaagggccagcccggcgacatctaccaccagacctgggcccggtacttcgtgaagttcctggacgcctatgccgagcacaagctgcagttctgggccgtgaccgccgagaatgagcctagcgctggactgctgtccggctaccccttccaatgcctgggcttcacacccgagcaccagcgggactttatcgccagagatctgggccccacactggccaacagcacccaccacaacgtgcggctgctgatgctggacgaccagagactgctgctcccccactgggccaaggtggtgctgacagaccccgaggccgccaaatacgtccacgggattgccgtgcactggtatctggactttctggcccctgccaaggccacactgggcgagacacaccggctgttccccaacaccatgctgttcgccagcgaggcctgcgtgggcagcaagttctgggagcagagcgtgcggctgggcagctgggacagaggcatgcagtacagccacagcatcatcaccaacctgctgtaccacgtcgtgggctggaccgactggaatctggccctgaaccctgagggaggacccaactgggtccgcaacttcgtggacagccccatcatcgtggacatcaccaaggacaccttctacaagcagcccatgttctaccacctgggccacttcagcaagttcatccccgagggcagccagagagtgggactggtcgccagccagaagaacgatctggacgccgtggccctgatgcaccctgatggcagcgctgtggtggtggtcctgaatcggtccagcaaggacgtgcccctgaccatcaaggaccccgccgtgggcttcctggaaaccatcagccccggctactccatccacacctacctgtggcggagacaatga

SEQ ID NO: 8 - DNA, 1869 bp, Artificial Sequence
  Synthetic Construct
atggaattcagcagccccagccgggaggaatgccccaagcccctgagcagagtgtccatcatggccggatctctgaccggactgctgctgctgcaagccgtgtcttgggccagcggcgccagaccttgcatccccaagagcttcggctacagcagcgtcgtgtgcgtgtgcaacgccacctactgcgacagcttcgacccccctacctttcccgccctgggcaccttcagcagatacgagagcacccgcagcggcagacggatggaactgagcatgggccccatccaggccaatcacaccggaaccggcctgctgctgaccctgcagcccgagcagaaattccagaaagtgaagggcttcggcggagccatgacagacgccgctgccctgaacatcctggccctgagcccccctgctcagaatctgctgctcaagagctacttcagcgaggaaggcatcggctacaacatcatccgggtgcccatggctagctgcgacttcagcatccggacctacacctacgccgacacccccgacgacttccagctgcacaacttcagcctgcccgaagaggacaccaagctgaagatccccctgatccacagagccctgcagctggcccagaggcctgtgtctctgctggctagcccttggaccagccccacctggctgaaaaccaacggagccgtgaacggcaagggcagcctgaagggccagcccggcgacatctaccaccagacctgggcccggtacttcgtgaagttcctggacgcctatgccgagcacaagctgcagttctgggccgtgaccgccgagaatgagcctagcgctggactgctgtccggctaccccttccaatgcctgggcttcacacccgagcaccagcgggactttatcgccagagatctgggccccacactggccaacagcacccaccacaacgtgcggctgctgatgctggacgaccagagactgctgctcccccactgggccaaggtggtgctgacagaccccgaggccgccaaatacgtccacgggattgccgtgcactggtatctggactttctggcccctgccaaggccacactgggcgagacacaccggctgttccccaacaccatgctgttcgccagcgaggcctgcgtgggcagcaagttctgggagcagagcgtgcggctgggcagctgggacagaggcatgcagtacagccacagcatcatcaccaacctgctgtaccacgtcgtgggctggaccgactggaatctggccctgaaccctgagggaggacccaactgggtccgcaacttcgtggacagccccatcatcgtggacatcaccaaggacaccttctacaagcagcccatgttctaccacctgggccacttcagcaagttcatccccgagggcagccagagagtgggactggtcgccagccagaagaacgatctggacgccgtggccctgatgcaccctgatggcagcgctgtggtggtggtcctgaatcggtccagcaaggacgtgcccctgaccatcaaggaccccgccgtgggcttcctggaaaccatcagccccggctactccatccacacctacctgtggcggagacaaggcggaggcggagctggtggcggcggagcaggcggtggtggtgcaggcggcggaggtgctggcggaggaccatctctttgtggcggagaactggtggacaccctgcagttcgtgtgtggcgacagaggcttctactttagcagacccgccagcagagtgtccgccagatctagaggaatcgtggaagagtgctgcttcagaagctgcgacctggcactgctggaaacctactgtgccacaccagccaagagcgagtgatga

SEQ ID NO: 9 - DNA, 1887 bp, Artificial Sequence
  Synthetic Construct
atggaattcagcagccccagccgggaggaatgccccaagcccctgagcagagtgtccatcatggccggatctctgaccggactgctgctgctgcaagccgtgtcttgggccagcggcgccagaccttgcatccccaagagcttcggctacagcagcgtcgtgtgcgtgtgcaacgccacctactgcgacagcttcgacccccctacctttcccgccctgggcaccttcagcagatacgagagcacccgcagcggcagacggatggaactgagcatgggccccatccaggccaatcacaccggaaccggcctgctgctgaccctgcagcccgagcagaaattccagaaagtgaagggcttcggcggagccatgacagacgccgctgccctgaacatcctggccctgagcccccctgctcagaatctgctgctcaagagctacttcagcgaggaaggcatcggctacaacatcatccgggtgcccatggctagctgcgacttcagcatccggacctacacctacgccgacacccccgacgacttccagctgcacaacttcagcctgcccgaagaggacaccaagctgaagatccccctgatccacagagccctgcagctggcccagaggcctgtgtctctgctggctagcccttggaccagccccacctggctgaaaaccaacggagccgtgaacggcaagggcagcctgaagggccagcccggcgacatctaccaccagacctgggcccggtacttcgtgaagttcctggacgcctatgccgagcacaagctgcagttctgggccgtgaccgccgagaatgagcctagcgctggactgctgtccggctaccccttccaatgcctgggcttcacacccgagcaccagcgggactttatcgccagagatctgggccccacactggccaacagcacccaccacaacgtgcggctgctgatgctggacgaccagagactgctgctcccccactgggccaaggtggtgctgacagaccccgaggccgccaaatacgtccacgggattgccgtgcactggtatctggactttctggcccctgccaaggccacactgggcgagacacaccggctgttccccaacaccatgctgttcgccagcgaggcctgcgtgggcagcaagttctgggagcagagcgtgcggctgggcagctgggacagaggcatgcagtacagccacagcatcatcaccaacctgctgtaccacgtcgtgggctggaccgactggaatctggccctgaaccctgagggaggacccaactgggtccgcaacttcgtggacagccccatcatcgtggacatcaccaaggacaccttctacaagcagcccatgttctaccacctgggccacttcagcaagttcatccccgagggcagccagagagtgggactggtcgccagccagaagaacgatctggacgccgtggccctgatgcaccctgatggcagcgctgtggtggtggtcctgaatcggtccagcaaggacgtgcccctgaccatcaaggaccccgccgtgggcttcctggaaaccatcagccccggctactccatccacacctacctgtggcggagacaaggagcaccaggcggaggatctccagctcctgctcctacaccagctccagcaccgacgcctgctccagctggcggaggaccttctggtgcacctctttgtggcggagagctggtggataccctgcagttcgtgtgtggcgaccggggcttctactttagcagacctgccagcagagtgtccgccagatctagaggcatcgtggaagagtgctgcttcagaagctgcgacctggcactgctggaaacctactgtgccacaccagccaagagcgagtgatga

SEQ ID NO: 10 - DNA, 1962 bp, Artificial Sequence
  Synthetic Construct
atggaattcagcagccccagccgggaggaatgccccaagcccctgagcagagtgtccatcatggccggatctctgaccggactgctgctgctgcaagccgtgtcttgggccagcggcgccagaccttgcatccccaagagcttcggctacagcagcgtcgtgtgcgtgtgcaacgccacctactgcgacagcttcgacccccctacctttcccgccctgggcaccttcagcagatacgagagcacccgcagcggcagacggatggaactgagcatgggccccatccaggccaatcacaccggaaccggcctgctgctgaccctgcagcccgagcagaaattccagaaagtgaagggcttcggcggagccatgacagacgccgctgccctgaacatcctggccctgagcccccctgctcagaatctgctgctcaagagctacttcagcgaggaaggcatcggctacaacatcatccgggtgcccatggctagctgcgacttcagcatccggacctacacctacgccgacacccccgacgacttccagctgcacaacttcagcctgcccgaagaggacaccaagctgaagatccccctgatccacagagccctgcagctggcccagaggcctgtgtctctgctggctagcccttggaccagccccacctggctgaaaaccaacggagccgtgaacggcaagggcagcctgaagggccagcccggcgacatctaccaccagacctgggcccggtacttcgtgaagttcctggacgcctatgccgagcacaagctgcagttctgggccgtgaccgccgagaatgagcctagcgctggactgctgtccggctaccccttccaatgcctgggcttcacacccgagcaccagcgggactttatcgccagagatctgggccccacactggccaacagcacccaccacaacgtgcggctgctgatgctggacgaccagagactgctgctcccccactgggccaaggtggtgctgacagaccccgaggccgccaaatacgtccacgggattgccgtgcactggtatctggactttctggcccctgccaaggccacactgggcgagacacaccggctgttccccaacaccatgctgttcgccagcgaggcctgcgtgggcagcaagttctgggagcagagcgtgcggctgggcagctgggacagaggcatgcagtacagccacagcatcatcaccaacctgctgtaccacgtcgtgggctggaccgactggaatctggccctgaaccctgagggaggacccaactgggtccgcaacttcgtggacagccccatcatcgtggacatcaccaaggacaccttctacaagcagcccatgttctaccacctgggccacttcagcaagttcatccccgagggcagccagagagtgggactggtcgccagccagaagaacgatctggacgccgtggccctgatgcaccctgatggcagcgctgtggtggtggtcctgaatcggtccagcaaggacgtgcccctgaccatcaaggaccccgccgtgggcttcctggaaaccatcagccccggctactccatccacacctacctgtggcggagacaaggagcaccaggcggatctccagcaggatctccaacctctaccgaggaaggcacaagcgagtctgccacacctgagtctggacctggcacaagcacagagcctagcgaaggatctgccccaggttctcctgccggctctcctacaagtacaggaccttctggcgctccactgtgtggcggagaactggtggataccctgcagttcgtgtgcggcgacagaggcttctactttagcagacccgccagcagagtgtccgccagatctagaggaatcgtggaagagtgctgcttcagaagctgcgatctggcactgctggaaacctactgtgccacaccagccaagagcgagtgatga

SEQ ID NO: 11 - DNA, 1566 bp, Artificial Sequence
  Synthetic Construct
atggggattcctatgggcaagtctatgctggtcctgctgacatttctggccttcgcttcatgctgtatcgctgccagaccttgcatccccaagagcttcggctacagcagcgtcgtgtgcgtgtgcaacgccacctactgcgacagcttcgacccccctacctttcccgccctgggcaccttcagcagatacgagagcacccgcagcggcagacggatggaactgagcatgggccccatccaggccaatcacaccggaaccggcctgctgctgaccctgcagcccgagcagaaattccagaaagtgaagggcttcggcggagccatgacagacgccgctgccctgaacatcctggccctgagcccccctgctcagaatctgctgctcaagagctacttcagcgaggaaggcatcggctacaacatcatccgggtgcccatggctagctgcgacttcagcatccggacctacacctacgccgacacccccgacgacttccagctgcacaacttcagcctgcccgaagaggacaccaagctgaagatccccctgatccacagagccctgcagctggcccagaggcctgtgtctctgctggctagcccttggaccagccccacctggctgaaaaccaacggagccgtgaacggcaagggcagcctgaagggccagcccggcgacatctaccaccagacctgggcccggtacttcgtgaagttcctggacgcctatgccgagcacaagctgcagttctgggccgtgaccgccgagaatgagcctagcgctggactgctgtccggctaccccttccaatgcctgggcttcacacccgagcaccagcgggactttatcgccagagatctgggccccacactggccaacagcacccaccacaacgtgcggctgctgatgctggacgaccagagactgctgctcccccactgggccaaggtggtgctgacagaccccgaggccgccaaatacgtccacgggattgccgtgcactggtatctggactttctggcccctgccaaggccacactgggcgagacacaccggctgttccccaacaccatgctgttcgccagcgaggcctgcgtgggcagcaagttctgggagcagagcgtgcggctgggcagctgggacagaggcatgcagtacagccacagcatcatcaccaacctgctgtaccacgtcgtgggctggaccgactggaatctggccctgaaccctgagggaggacccaactgggtccgcaacttcgtggacagccccatcatcgtggacatcaccaaggacaccttctacaagcagcccatgttctaccacctgggccacttcagcaagttcatccccgagggcagccagagagtgggactggtcgccagccagaagaacgatctggacgccgtggccctgatgcaccctgatggcagcgctgtggtggtggtcctgaatcggtccagcaaggacgtgcccctgaccatcaaggaccccgccgtgggcttcctggaaaccatcagccccggctactccatccacacctacctgtggcggagacaatga

SEQ ID NO: 12 - protein, 24 aa, Artificial Sequence
  Synthetic Construct
MGIPMGKSMLVLLTFLAFASCCIA

SEQ ID NO: 13 - DNA, 72 bp, Artificial Sequence
  Synthetic Construct
atggggattcctatgggcaagtctatgctggtcctgctgacatttctggccttcgcttcatgctgtatcgct

SEQ ID NO: 14 - protein, 477 aa, Homo sapiens
MGRCCFYTAGTLSLLLLVTSVTLLVARVFQKAVDQSIEKKIVLRNGTEAFDSWEKPPLPVYTQFYFFNVTNPEEILRGETPRVEEVGPYTYRELRNKANIQFGDNGTTISAVSNKAYVFERDQSVGDPKIDLIRTLNIPVLTVIEWSQVHFLREIIEAMLKAYQQKLFVTHTVDELLWGYKDEILSLIHVFRPDISPYFGLFYEKNGTNDGDYVFLTGEDSYLNFTKIVEWNGKTSLDWWITDKCNMINGTDGDSFHPLITKDEVLYVFPSDFCRSVYITFSDYESVQGLPAFRYKVPAELANTSDNAGFCIPEGNCLGSGVLNVSICKNGAPIIMSFPHFYQADERFVSAIEGMHPNQEDHETFVDINPLTGIILKAAKRFQINIYVKKLDDFVETGDIRTMVFPVMYLNESVHIDKETASRLKSMINTTLIITNIPYIIMALGVFFGLVFTWLACKGQGSMDEGTADERAPLIRT

SEQ ID NO: 15 - protein, 335 aa, Homo sapiens
MGRCCFYTAGTLSLLLLVTSVTLLVARVFQKAVDQSIEKKIVLRNGTEAFDSWEKPPLPVYTQFYFFNVTNPEEILRGETPRVEEVGPYTYRSLDWWITDKCNMINGTDGDSFHPLITKDEVLYVFPSDFCRSVYITFSDYESVQGLPAFRYKVPAEILANTSDNAGFCIPEGNCLGSGVLNVSICKNGAPIIMSFPHFYQADERFVSAIEGMHPNQEDHETFVDINPLTGIILKAAKRFQINIYVKKLDDFVETGDIRTMVFPVMYLNESVHIDKETASRLKSMINTTLIITNIPYIIMALGVFFGLVFTWLACKGQGSMDEGTADERAPLIRT

SEQ ID NO: 16 - DNA, 4780 bp, Homo sapiens
gcagtcgccggcggttgcctcgcggggttgcggcgagcccggcccgcgaacgtcacgtccctgcgcgctccctgcactctcccgagctgcgctaggcgggcgccacggctgcccggcgaaggaaaccgaaaccgagtccgggcccgtccctccgcggccccatccgcccggtgcacccggggccgcgctcgccaggccgcggagcccagagctgcgcgcacgaaccgtgcgccgggagggcgtgggcgtggcgccgaagggtcccgggtcttcgacgcctctgcggcggctcctccctccttgcagttggatccctggcgggtgcggcccggcccggcccgtgagcggcgcacagaatgggccgatgctgcttctacacggcggggacgttgtccctgctcctgctggtgaccagcgtcacgctgctggtggcccgggtcttccagaaggctgtagaccagagtatcgagaagaaaattgtgttaaggaatggtactgaggcatttgactcctgggagaagccccctctgcctgtgtatactcagttctatttcttcaatgtcaccaatccagaggagatcctcagaggggagacccctcgggtggaagaagtggggccatacacctacagggaactcagaaacaaagcaaatattcaatttggagataatggaacaacaatatctgctgttagcaacaaggcctatgtttttgaacgagaccaatctgttggagaccctaaaattgacttaattagaacattaaatattcctgtattgactgtcatagagtggtcccaggtgcacttcctcagggagatcatcgaggccatgttgaaagcctatcagcagaagctctttgtgactcacacagttgacgaattgctctggggctacaaagatgaaatcttgtcccttatccatgttttcaggcccgatatctctccctattttggcctattctatgagaaaaatgggactaatgatggagactatgtttttctaactggagaagacagttaccttaactttacaaaaattgtggaatggaatgggaaaacgtcacttgactggtggataacagacaagtgcaatatgattaatggaacagatggagattcttttcacccactaataaccaaagatgaggtcctttatgtcttcccatctgacttttgcaggtcagtgtatattactttcagtgactatgagagtgtacagggactgcctgcctttcggtataaagttcctgcagaaatattagccaatacgtcagacaatgccggcttctgtatacctgagggaaactgcctgggctcaggagttctgaatgtcagcatctgcaagaatggtgcacccatcattatgtctttcccacacttttaccaagcagatgagaggtttgtttctgccatagaaggcatgcacccaaatcaggaagaccatgagacatttgtggacattaatcctttgactggaataatcctaaaagcagccaagaggttccaaatcaacatttatgtcaaaaaattagatgactttgttgaaacgggagacattagaaccatggttttcccagtgatgtacctcaatgagagtgttcacattgataaagagacggcgagtcgactgaagtctatgattaacactactttgatcatcaccaacataccctacatcatcatggcgctgggtgtgttctttggtttggtttttacctggcttgcatgcaaaggacagggatccatggatgagggaacagcggatgaaagagcacccctcattcgaacctaaacattgcctttgcttggtgaagaaactgtgtgagctgtcctgacctggacgatgacgtggggaaaccctccacctccttgcaggcttgttgcctgttgaaagaaggaaaaagacacggcgctggcaagtgataggaacattctggccagaggttaaagagcaggctgacatggctggccattaagctttataaaatcatgtgggctctgaaattgttcttttatgtgtctagcaagtatttaataaacccttgtatagtaattttgttgttgttgggtgctggtagctccagaattttgtgaccactattgtgggtaaaatgtctctgcatcacttgttaatgctactggtctaacttcattcagtatgcttcattcaccgaactttgtgctcaaaatgcgtatataccattttatgttgtattcctccatttcacttgcaaaacagaagtaaataagagttcgggacccagggtaaaatggtagcttcatccaatatatcattcaaatgcatctgatttctaaaacatattacattttatgctgatcttcagttcataattcttccaggaaaactcagtcttccaactgcaataaaatactgggtagaatcaaatgggaaaggggttgggtggggcaatacccatgagttgatagtgataagctcctaaggatttttaacttgtacttttgtgaacgaagagaatgcataaataatgttggtgaggataaagtacagatatttcatgtagaattaattgctagttatgatgcttgtggatagttaactgttttttttttagtcaaaatgatcatgctacgaaaagatgcttctgagagaatgtaatgagtaactgatttttcttcctgagtcgcccttgccaaatatgttactgtattaattaatctaatattgagtgattatttgtaaaattatgaatatgggaaatccatctatctacagcctaagttacacataagtttcagaaagtctgattagactaaagagatatttcttctgggacagccgccttcttggtaattttgaagttctttttacaagttccttcctcagtttcagttctttccagtgttttgtagctcactgtcactcactgaatagagaaacgtgtgccctatacttcctgtgacaatcattttgctgacagaatgatggatgtttaaaatattgcacaaagtactttaaagaaaggtctgttaggaccagaagcagagacaccacttttcaaaggacttcttggtttcagcataacctaagacagggaattgggagccatcatatgtcacagtgttcagaattcaagcatatttaagggcattttctttgattctcaaagttcagcattcattttgaattgagaagcctatacatttagctgacaaagtgcttatagaatttcttaacaactgaaccattcaaaaggattttttttgtttaaaactggatttcaatgtaagcaaatgaagaaaaaaatatagatttcatttccatagcttcttatccctgtattgaggtaataaattgttttactgacaatttttcctttttctacactaaaacaatatgtgatatatttcccctcttgaagaggcaattcattaaactctcaaattttctatagaatcaagatagaacctttagatactccaactcaccaaaatgtaaaaaaactaacaaaaatatttggtcttcaataatgctaaatatctacatttttagaatttatcaacatttaactagataattgggcatgtcttaattatgcatgtacttatccatactaataaaattgacaatgctagtgcatacttattggtttagtcctattatcaggatataatcatctgtgaggaggatattttaaatactgtaaatgataacagttaatgatatacacatttagactgagttgcacactggcagggagaccaaaaacattacttccatacttgtgtcatgattcttttttttttgagagagtctcactctgtcgccaggctggagtacagtggcatgatctcggctcactgcaacctctgcctcccgggttcaagcaattctcctgcctcagccacccaagtagctgggactacaggtgcgtgccaccacgcccagctaaattttgtatttttagtggagacggggtttcaccatgttggccaggatggtctcaatctcctgaccctgcgatctgcccacctcagcctcccaaagtgctgggattacaggcgtaagccaccgggcctggcctgttttatgattcttaatagttacttggtttaaatcacatttgatactatccttctgaaaagtctgagacagatctacaaactacagtcaaaattatagattaagaggaatgaatgcacctatttggctttaagttgaagatgaattatttctcatgctcattttcttgcggcagttatcttagaaagacccccaaaggctttgtgattgtaagcactgtcatgatcacagaatgcaagcttctggtaccatgatcctcaacttagagaggaagaaaccaagacagagagcttaactcacttctctcagggaaaattaggagttgagcacaggacaggaaatgggctttgccacttttagctccaggcttttctaaccagacttgatttcctcatgttctagaaagatcactaatggtcaagtggaacaagcactacacgactaacccctattggggtttttaacttaagggaggctaatttttaatttaaactgctcgagatatgagttctgcaaaaggtggtccgcatccttggccctctggacattatcactaaattgcttgtgcctgttaacaagaatactgaccagaatgctcttcatgtagcttatacagttggttcacttcatgcggttcttgacatgtttatttctacccttaatgcaatgaaatgtttcattaataaaaaaccactttatataaaaaaaaaaaaaaa

SEQ ID NO: 17 - DNA, 4340 bp, Homo sapiens
gcagtcgccggcggttgcctcgcggggttgcggcgagcccggcccgcgaacgtcacgtccctgcgcgctccctgcactctcccgagctgcgctaggcgggcgccacggctgcccggcgaaggaaaccgaaaccgagtccgggcccgtccctccgcggccccatccgcccggtgcacccggggccgcgctcgccaggccgcggagcccagagctgcgcgcacgaaccgtgcgccgggagggcgtgggcgtggcgccgaagggtcccgggtcttcgacgcctctgcggcggctcctccctccttgcagttggatccctggcgggtgcggcccggcccggcccgtgagcggcgcacagaatgggccgatgctgcttctacacggcggggacgttgtccctgctcctgctggtgaccagcgtcacgctgctggtggcccgggtcttccagaaggctgtagaccagagtatcgagaagaaaattgtgttaaggaatggtactgaggcatttgactcctgggagaagccccctctgcctgtgtatactcagttctatttcttcaatgtcaccaatccagaggagatcctcagaggggagacccctcgggtggaagaagtggggccatacacctacaggtcacttgactggtggataacagacaagtgcaatatgattaatggaacagatggagattcttttcacccactaataaccaaagatgaggtcctttatgtcttcccatctgacttttgcaggtcagtgtatattactttcagtgactatgagagtgtacagggactgcctgcctttcggtataaagttcctgcagaaatattagccaatacgtcagacaatgccggcttctgtatacctgagggaaactgcctgggctcaggagttctgaatgtcagcatctgcaagaatggtgcacccatcattatgtctttcccacacttttaccaagcagatgagaggtttgtttctgccatagaaggcatgcacccaaatcaggaagaccatgagacatttgtggacattaatcctttgactggaataatcctaaaagcagccaagaggttccaaatcaacatttatgtcaaaaaattagatgactttgttgaaacgggagacattagaaccatggttttcccagtgatgtacctcaatgagagtgttcacattgataaagagacggcgagtcgactgaagtctatgattaacactactttgatcatcaccaacataccctacatcatcatggcgctgggtgtgttctttggtttggtttttacctggcttgcatgcaaaggacagggatccatggatgagggaacagcggatgaaagagcacccctcattcgaacctaaacattgcctttgcttggtgaagaaactgtgtgagctgtcctgacctggacgatgacgtggggaaaccctccacctccttgcaggcttgttgcctgttgaaagaaggaaaaagacacggcgctggcaagtgataggaacattctggccagaggttaaagagcaggctgacatggctggccattaagctttataaaatcatgtgggctctgaaattgttcttttatgtgtctagcaagtatttaataaacccttgtatagtaattttgttgttgttgggtgctggtagctccagaattttgtgaccactattgtgggtaaaatgtctctgcatcacttgttaatgctactggtctaacttcattcagtatgcttcattcaccgaactttgtgctcaaaatgcgtatataccattttatgttgtattcctccatttcacttgcaaaacagaagtaaataagagttcgggacccagggtaaaatggtagcttcatccaatatatcattcaaatgcatctgatttctaaaacatattacattttatgctgatcttcagttcataattcttccaggaaaactcagtcttccaactgcaataaaatactgggtagaatcaaatgggaaaggggttgggtggggcaatacccatgagttgatagtgataagctcctaaggatttttaacttgtacttttgtgaacgaagagaatgcataaataatgttggtgaggataaagtacagatatttcatgtagaattaattgctagttatgatgcttgtggatagttaactgttttttttttagtcaaaatgatcatgctacgaaaagatgcttctgagagaatgtaatgagtaactgatttttcttcctgagtcgcccttgccaaatatgttactgtattaattaatctaatattgagtgattatttgtaaaattatgaatatgggaaatccatctatctacagcctaagttacacataagtttcagaaagtctgattagactaaagagatatttcttctgggacagccgccttcttggtaattttgaagttctttttacaagttccttcctcagtttcagttctttccagtgttttgtagctcactgtcactcactgaatagagaaacgtgtgccctatacttcctgtgacaatcattttgctgacagaatgatggatgtttaaaatattgcacaaagtactttaaagaaaggtctgttaggaccagaagcagagacaccacttttcaaaggacttcttggtttcagcataacctaagacagggaattgggagccatcatatgtcacagtgttcagaattcaagcatatttaagggcattttctttgattctcaaagttcagcattcattttgaattgagaagcctatacatttagctgacaaagtgcttatagaatttcttaacaactgaaccattcaaaaggattttttttgtttaaaactggatttcaatgtaagcaaatgaagaaaaaaatatagatttcatttccatagcttcttatccctgtattgaggtaataaattgttttactgacaatttttcctttttctacactaaaacaatatgtgatatatttcccctcttgaagaggcaattcattaaactctcaaattttctatagaatcaagatagaacctttagatactccaactcaccaaaatgtaaaaaaactaacaaaaatatttggtcttcaataatgctaaatatctacatttttagaatttatcaacatttaactagataattgggcatgtcttaattatgcatgtacttatccatactaataaaattgacaatgctagtgcatacttattggtttagtcctattatcaggatataatcatctgtgaggaggatattttaaatactgtaaatgataacagttaatgatatacacatttagactgagttgcacactggcagggagaccaaaaacattacttccatacttgtgtcatgattcttttttttttgagagagtctcactctgtcgccaggctggagtacagtggcatgatctcggctcactgcaacctctgcctcccgggttcaagcaattctcctgcctcagccacccaagtagctgggactacaggtgcgtgccaccacgcccagctaaattttgtatttttagtggagacggggtttcaccatgttggccaggatggtctcaatctcctgaccctgcgatctgcccacctcagcctcccaaagtgctgggattacaggcgtaagccaccgggcctggcctgttttatgattcttaatagttacttggtttaaatcacatttgatactatccttctgaaaagtctgagacagatctacaaactacagtcaaaattatagattaagaggaatgaatgcacctatttggctttaagttgaagatgaattatttctcatgctcattttcttgcggcagttatcttagaaagacccccaaaggctttgtgattgtaagcactgtcatgatcacagaatgcaagcttctggtaccatgatcctcaacttagagaggaagaaaccaagacagagagcttaactcacttctctcagggaaaattaggagttgagcacaggacaggaaatgggctttgccacttttagctccaggcttttctaaccagacttgatttcctcatgttctagaaagatcactaatggtcaagtggaacaagcactacacgactaacccctattggggtttttaacttaagggaggctaatttttaatttaaactgctcgagatatgagttctgcaaaaggtggtccgcatccttggccctctggacattatcactaaattgcttgtgcctgttaacaagaatactgaccagaatgctcttcatgtagcttatacagttggttcacttcatgcggttcttgacatgtttatttctacccttaatgcaatgaaatgtttcattaataaaaaaccactttatataaaa

SEQ ID NO: 18 - protein, 16 aa, Homo sapiens
LREIIEAMLKAYQQKL

SEQ ID NO: 19 - protein, 37 aa, Homo sapiens
IIEAMLKAYQQKLFVTHTVDELLWGYKDEILSLIHVF

SEQ ID NO: 20 - DNA, 385 bp, Artificial Sequence
  Synthetic Construct
gatcaaggttaggaacagagagacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgg

SEQ ID NO: 21 - DNA, 398 bp, Artificial Sequence
  Synthetic Construct
ttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 22 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 23 - protein, 85 aa, Artificial Sequence
  Synthetic Construct
GGGGAGGGGAGGGGAGGGGAGGGPSLCGGELVDTLQFVCGDRGFYFSRPASRVSARSRGIVEECCFRSCDLALLETYCATPAKSE

SEQ ID NO: 24 - protein, 91 aa, Artificial Sequence
  Synthetic Construct
GAPGGGSPAPAPTPAPAPTPAPAGGGPSGAPLCGGELVDTLQFVCGDRGFYFSRPASRVSARSRGIVEECCFRSCDLALLETYCATPAKSE

SEQ ID NO: 25 - protein, 116 aa, Artificial Sequence
  Synthetic Construct
GAPGGSPAGSPTSTEEGTSESATPESGPGTSTEPSEGSAPGSPAGSPTSTGPSGAPLCGGELVDTLQFVCGDRGFYFSRPASRVSARSRGIVEECCFRSCDLALLETYCATPAKSE

SEQ ID NO: 26 - DNA, 260 bp, Artificial Sequence
  Synthetic Construct
ggcggaggcggagctggtggcggcggagcaggcggtggtggtgcaggcggcggaggtgctggcggaggaccatctctttgtggcggagaactggtggacaccctgcagttcgtgtgtggcgacagaggcttctactttagcagacccgccagcagagtgtccgccagatctagaggaatcgtggaagagtgctgcttcagaagctgcgacctggcactgctggaaacctactgtgccacaccagccaagagcgagtgatg

SEQ ID NO: 27 - DNA, 279 bp, Artificial Sequence
  Synthetic Construct
ggagcaccaggcggaggatctccagctcctgctcctacaccagctccagcaccgacgcctgctccagctggcggaggaccttctggtgcacctctttgtggcggagagctggtggataccctgcagttcgtgtgtggcgaccggggcttctactttagcagacctgccagcagagtgtccgccagatctagaggcatcgtggaagagtgctgcttcagaagctgcgacctggcactgctggaaacctactgtgccacaccagccaagagcgagtgatga

SEQ ID NO: 28 - DNA, 354 bp, Artificial Sequence
  Synthetic Construct
ggagcaccaggcggatctccagcaggatctccaacctctaccgaggaaggcacaagcgagtctgccacacctgagtctggacctggcacaagcacagagcctagcgaaggatctgccccaggttctcctgccggctctcctacaagtacaggaccttctggcgctccactgtgtggcggagaactggtggataccctgcagttcgtgtgcggcgacagaggcttctactttagcagacccgccagcagagtgtccgccagatctagaggaatcgtggaagagtgctgcttcagaagctgcgatctggcactgctggaaacctactgtgccacaccagccaagagcgagtgatga

SEQ ID NO: 29 - protein, 317 aa, Artificial Sequence [sequence omitted]

SEQ ID NO: 30 - protein, 15 aa, Artificial Sequence
  Synthetic Construct
RQIKWFQNRRMKWKK

SEQ ID NO: 31 - protein, 11 aa, Artificial Sequence
  Synthetic Construct
YGRKKRRQRRR

SEQ ID NO: 32 - protein, 18 aa, Artificial Sequence
  Synthetic Construct
RGGRLSYSRRRFSTSTGR

SEQ ID NO: 33 - protein, 10 aa, Artificial Sequence
  Synthetic Construct
RRLSYSRRRF

SEQ ID NO: 34 - protein, 12 aa, Artificial Sequence
  Synthetic Construct
PIRRRKKLRRLK

SEQ ID NO: 35 - protein, 12 aa, Artificial Sequence
  Synthetic Construct
RRQRRTSKLMKR

SEQ ID NO: 36 - protein, 15 aa, Artificial Sequence
  Synthetic Construct
RRRRNRTRRNRRRVR

SEQ ID NO: 37 - protein, 19 aa, Artificial Sequence
  Synthetic Construct
KMTRAQRRAAARRNRWTAR

SEQ ID NO: 38 - protein, 13 aa, Artificial Sequence
  Synthetic Construct
TRRQRTRRARRNR

SEQ ID NO: 39 - protein, 13 aa, Artificial Sequence
  Synthetic Construct
GRKKRRQRRRPPQ

SEQ ID NO: 40 - protein, 13 aa, Artificial Sequence
  Synthetic Construct
GRRRRRRRRRPPQ

SEQ ID NO: 41 - protein, 27 aa, Artificial Sequence
  Synthetic Construct
GWTLNSAGYLLGKINLKALAALAKKIL

SEQ ID NO: 42 - protein, 17 aa, Artificial Sequence
  Synthetic Construct
KLALKLALKLALALKLA

SEQ ID NO: 43 - protein, 27 aa, Artificial Sequence
  Synthetic Construct
MGLGLHLLVLAAALQGAWSQPKKKRKV

SEQ ID NO: 44 - protein, 27 aa, Artificial Sequence
  Synthetic Construct
GALFLGWLGAAGSTMGAWSQPKKKRKV

SEQ ID NO: 45 - protein, 27 aa, Artificial Sequence
  Synthetic Construct
GALFLGFLGAAGSTMGAWSQPKKKRKV

SEQ ID NO: 46 - protein, 27 aa, Artificial Sequence
  Synthetic Construct
GALFLGFLGAAGSTMGAWSQPKSKRKV

SEQ ID NO: 47 - protein, 21 aa, Artificial Sequence
  Synthetic Construct
KETWWETWWTEWSQPKKKRKV

SEQ ID NO: 48 - protein, 21 aa, Artificial Sequence
  Synthetic Construct
KETWFETWFTEWSQPKKKRKV

SEQ ID NO: 49 - protein, 26 aa, Homo sapiens
MGRCCFYTAGTLSLLLLVTSVTLLVA